(19) United States
(12) Patent Application Publication
(10) Pub. No.: US 2016/0008785 A1
(43) Pub. Date: Jan. 14, 2016

(54) DEVICES AND METHODS FOR PRODUCING AND ANALYZING MICROARRAYS

(71) Applicant: Adeptrix Corp., Beverly, MA (US)

(72) Inventor: Vladislav B. Bergo, Boston, MA (US)

(21) Appl. No.: 14/795,346

(22) Filed: Jul. 9, 2015

Related U.S. Application Data
(63) Continuation of application No. 13/369,939, filed on Feb. 9, 2012.
(60) Provisional application No. 61/441,069, filed on Feb. 9, 2011.
(60) Provisional application No. 61/488,443, filed on May 20, 2011.
(60) Provisional application No. 61/554,183, filed on Nov. 1, 2011.
(60) Provisional application No. 61/562,239, filed on Nov. 21, 2011.

(51) Int. Cl. B01J 19/00; G01N 33/68

(52) U.S. Cl. CPC B01J 19/0046; G01N 33/6848; B01J 2219/00725; B01J 2219/00756; B01J 2219/00648; B01J 2219/00655; B01J 2219/00596; B01J 2219/00585; B01J 2219/00709

(57) ABSTRACT

Devices and methods for producing and analyzing microarrays are disclosed. In an embodiment, a method for converting a library of beads to an array of analytes includes positioning a plurality of beads having one or more analytes bound therein on a solid support in a spatially separated manner, causing the analytes to be released from the plurality of microparticles, and localizing the released analytes in discrete spots.

### RELATED APPLICATIONS

This application is a continuation of U.S. patent application Ser. No. 13/369,939, inventor Vladislav B. Bergo, filed Feb. 9, 2012, which, in turn, claims the benefit of and priority to U.S. Provisional Patent Application No. 61/441,069, filed on Feb. 9, 2011; U.S. Provisional Patent Application No. 61/488,443, filed on May 20, 2011; U.S. Provisional Patent Application No. 61/554,183, filed on Nov. 1, 2011; and U.S. Provisional Patent Application No. 61/562,239, filed on Nov. 21, 2011. The entirety of each of these applications is hereby incorporated herein by reference for the teachings therein.

### FIELD

The embodiments disclosed herein relate generally to the field of high-throughput biological assays and more specifically to the field of random bead arrays and processes for producing microarrays. The embodiments disclosed herein also relate to the field of acquisition and analysis of microarray data.

### BACKGROUND

Microarrays, due to their flexible design, high degree of multiplexing and ability to perform measurements in miniature format, are the preferred method of analysis in biological studies requiring screening of large numbers of samples.

Biological microarray technology was originally used for the analysis of oligonucleotides. Subsequently this approach was extended to other biomolecules, e.g. polypeptides, carbohydrates, lipids and small molecules. Other examples of microarrays include tissue and cell arrays.

The traditional microarray format requires that each capture reagent (also known as a probe) is immobilized on the surface of a microarray slide at a specific position, known as a spot. The two-dimensional coordinates of each spot determine the identity of the probe at that position. Consequently, the identity of a sample that interacts with each probe, often referred to as the target, is determined based of the specificity of the probe/target interaction. Microarrays of this type are referred to as ordered arrays or printed arrays. The unambiguous correlation between identity of the probe and its location on the microarray slide is known as positional encoding.

Alternative microarray layouts have been developed in which the identity of the probe cannot be inferred from its location on the array. Such microarrays are known as random arrays. An example of a commercially available random array is Illumina's® Bead Array, where individual microbeads are deposited into wells developed on the surface of a microarray slide. In this configuration the identity of the sample is determined using bead encoding, i.e. each bead carries a unique identifying label. A variety of bead encoding technologies are currently known. Numerous methods of optical encoding exist that include, for example, optical barcoding and combinations of fluorescent dyes.

Instead of being loaded on the solid support, beads and bead-bound analytes can be also measured in solution by flow cytometry. This technique is commercialized in several applications including the Luminex® platform.

Bead-based analytical platforms are commonly used to measure affinity interactions. In the most basic form of affinity assay, each bead carries a capture reagent and a bead label (bead tag). The bead label is reversibly or irreversibly linked to the bead. The capture reagent, or the probe, is a specific molecule or a molecular complex that has affinity for another molecule or molecular complex, which is known as the target. Multiple identical copies of the capture reagent are attached to each bead. The identical beads within the bead library, which carry the same capture reagent, are known as replicates. The binding of target to the probe is performed by incubation of a bead library with a medium containing target molecules, followed by washing to reduce the non-specific binding. The target molecules can be detected directly or by using a secondary probe, such as an antibody and, in some cases, an additional probe, such as a secondary antibody. By using libraries with different affinity beads, multiple targets can be captured in a single reaction, which is known as multiplexing. Fluorescence is widely used as a method of target detection.

In addition to probing affinity interactions, bead-based analytical technologies can be used to measure biomolecular reactions between enzymes and their corresponding substrates. In this approach, modification of the structure of bead-bound substrate by an enzyme is measured in order to identify the enzyme targets. Numerous assays have been developed that detect activity of a specific class of enzymes, e.g., kinases, phosphatases, proteases, etc.

Individual biomolecules and molecular complexes conjugated to beads or other microparticles may be used in many other biomedical applications. For example, micro- and nanoparticles may serve as drug-delivery vehicles that guide their cargo towards a specific group of cells, a tissue or an organ.

Consequently, there is a significant need to improve existing methods of measuring analytes bound to microparticles, for example to develop better analytical high-throughput screening platforms or to perform rapid QC of fabricated microparticle-conjugated molecular constructs.

The analytes are usually measured while still attached to their respective microbeads. This severely limits the range of analytical methods, which can be used to perform the assay readout. In fact, the majority of current readout methods utilize various forms of optical detection, such as fluorescence and luminescence and also radioactivity. On the other hand, mass spectrometry-based methods, which require ionization of the analyte, are rarely used in high-throughput bead assays. Yet, it is highly desirable to measure analytes in hundreds of thousands of individual mass channels by mass spectrometry in contrast to only a few channels available with optical detection. For example, in proteomics MS readout can be used to perform label-free detection, screen for protein post-translational modifications and obtain sequence information directly from analytes on individual beads.

While methods are known that achieve release (elution) of analytes from individual microbeads, they are either entirely manual, or limited to relatively small bead libraries. However bead libraries may contain hundreds of thousands or even millions of members. Furthermore, individual analytes conjugated to the same microbead may have different properties and furthermore, analytes may be attached to beads by linkages of the same or different nature. Accordingly, there is still a need for methods for analyzing bead libraries.

### SUMMARY

Devices and methods for producing and analyzing microarrays are disclosed herein. According to aspects illustrated herein, there is provided a method for converting a library of beads to an array of analytes that includes positioning a plurality of beads having one or more analytes bound therein on a solid support in a spatially separated manner, causing the analytes to be released from the plurality of microparticles, and localizing the released analytes in discrete spots.

According to aspects illustrated herein, there is provided a method for analyte analysis by mass spectrometry that includes converting a library of beads to an array of spots on a solid support, wherein each spot includes one or more analytes previously bound to a bead from the library of beads, and acquiring mass spectrometric data from the array of microspots according to a data acquisition protocol.

According to aspects illustrated herein, there is provided a device for analysis of analyte-conjugated beads that includes a solid support having a plurality of microwells arranged in a regular grid, wherein the microwells are sized to accept one or more beads with analytes conjugated thereto, and wherein the microwells are positioned at a predetermined distance from one another such that analytes released from the beads are localized in vicinity of respective beads.

### DESCRIPTION OF FIGURES

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments. The relationship between dimensions of the individual features, such as microbeads, microwells and spots of analytes, as depicted in the drawings, is only approximate. Instead, a range of suitable dimensions is provided within the text of the present specification.

FIG. 1 illustrates the steps of an embodiment process of fabricating an array of microspots from a bead library.

FIG. 2A is a schematic representation of an embodiment of a microwell array plate also showing microbeads deposited inside individual microbeads.

FIG. 2B is a schematic representation of a cross-section of the embodiment microwell array plate shown in FIG. 2A.

FIG. 3A, FIG. 3B, and FIG. 3C illustrate the steps an embodiment process of producing an array of microspots from a bead library.

FIG. 4 is an image of a section of an embodiment microwell array plate that can be used to perform mass spectrometry and fluorescence imaging.

FIG. 5 is a table listing some possible types of analyte-bead linkages and appropriate elution mechanisms.

FIGS. 6A-FIG. 6F illustrate an embodiment method of fabricating microspots of eluted analytes using solid phase MALDI matrix or nanoparticles.

FIG. 7 is a schematic illustration of an embodiment microwell design that can enhance elution of analytes from microbeads in the microwell array format.

FIG. 8A, FIG. 8B and FIG. 8C show detection (readout) channels of analyte-bead constructs by optical spectroscopy and mass spectrometry.

FIG. 9 illustrates a relationship between individual elements of the optical and mass spectrometric analysis of microarrays of the present disclosure.

FIG. 10A and FIG. 10B schematically depict an embodiment method steps for fabrication of a microarray system comprising arrays of microbeads and arrays of microspots.

FIG. 11 is a general depiction of analytes, which may be present on microbeads used for fabrication of an array of microspots.

FIG. 12A, FIG. 12B, and FIG. 12C demonstrate a relationship between the diameter of an analyte spot and the diameter of an instrument ionization beam.

FIG. 13A and FIG. 13B demonstrate a relationship between the diameter and displacement of the instrument ionization beam during the MS measurement.

FIG. 14A, FIG. 14B, and FIG. 14C demonstrate various options of the microarray scanning using MS.

FIGS. 15A-15E demonstrates various options of the mass channel and mass range selection for the visualization of microarray MS data.

FIG. 16A, FIG. 16B, and FIG. 16C demonstrate an the principle of microarray MS data analysis using a subsection of a microarray (FIG. 16A, 16B) and entire microarray area (FIG. 16C).

FIG. 17 demonstrates an example of an algorithm used to identify an unknown analyte in the microarray MS format.

FIG. 18A, FIG. 18B, FIG. 18C, and FIG. 18D demonstrate an embodiment of a method of analyte quantitation using signal from the target analyte, which is performed in the microarray MS format.

FIG. 19A, FIG. 19B, FIG. 19C, and FIG. 19D demonstrate an embodiment of a label-based method of analyte quantitation in the microarray MS format.

FIG. 20A, FIG. 20B, FIG. 20C, and FIG. 20D demonstrate an embodiment of a label-based method of analyte quantitation additionally including a control analyte.

FIG. 21A, FIG. 21B, FIG. 21C, and FIG. 21D demonstrate an embodiment of a method of analyte quantitation using signal from the target analyte and additionally including a control analyte.

FIG. 22A, FIG. 22B and FIG. 22C demonstrate an embodiment of a method of measuring analyte modification in a microarray MS format.

FIG. 23A and FIG. 23B illustrate the use of dual optical and mass spectrometric readout from a combination of a bead array and a microspot array.

FIG. 24 presents a MALDI TOF MS image of a polypeptide deposited on the surface of a microarray plate by elution from microbeads.

FIG. 25A shows representative single spot mass spectra obtained from: (1) an area of the array with loaded beads where no UV irradiation was applied; (2) an area of the array with loaded beads, which was exposed to UV irradiation for 5 minutes; (3) an area of the array devoid of beads, which was exposed to UV irradiation for 5 minutes.

FIG. 25B presents a MALDI TOF MS image of an array of analytes produced by UV photoelution.

FIG. 26A, FIG. 26B, and FIG. 26C present a MALDI TOF MS image of a bead array comprising only positive beads (FIG. 26A) and a mixture of positive/negative beads (FIG. 26B). A representative mass spectrum obtained from a positive bead (FIG. 26C).

FIG. 27 presents a MALDI TOF MS image of an approximately 10,000-member bead library loaded on the microwell array plate.

FIG. 28 presents an example of uniform microarray spots produced by UV photorelease of analytes from individual beads.

FIG. 29A and FIG. 29B present fluorescence (FIG. 29A) and MALDI TOF MS (FIG. 29B) images of a fluorescently labeled polypeptide eluted from beads.

FIG. 30A and FIG. 30B present fluorescence (FIG. 30A) image of the fluorescent label attached to beads and MALDI TOF MS (FIG. 30B) image of a polypeptide eluted from the same beads.

FIG. 31A and FIG. 31B present fluorescence (FIG. 31A) and MALDI TOF MS (FIG. 31B) images of fluorescent and polypeptide analytes, respectively, co-eluted from the same bead.

FIG. 32A, FIG. 32B, and FIG. 32C present fluorescence image of the analyte migration from beads arrayed on the microwell array plate.

FIG. 33 presents fluorescence image of the analyte bound to beads arrayed on the microwell array plate.

FIG. 34A, FIG. 34B, and FIG. 34C depict MALDI TOF MS analysis of the protein digest performed on beads arrayed on the microwell array slide.

FIG. 35A, FIG. 35B, FIG. 35C and FIG. 35D depict MALDI TOF MS detection of multiple analytes co-eluted from the same bead.

FIG. 36A, FIG. 36B, FIG. 36C and FIG. 36D depict MALDI TOF MS analysis of beads with two types of analytes attached via linkages of substantially different nature.

FIG. 37A, FIG. 37B, and FIG. 37C are a schematic representation of a microwell array plate with different well depth relative to the bead diameter.

FIG. 38A and FIG. 38B present MALDI TOF MS images of microarrays produced by releasing the analyte using UV illumination from beads loaded into microwells of different depth.

FIGS. 39A-39F present MALDI TOF MS images of microarrays produced by releasing the analyte using trypsin from beads loaded into microwells of different depth.

FIG. 40A and FIG. 40B illustrate fluorescence and MALDI TOF MS detection of analytes from beads smaller than 34 micron.

FIG. 41 presents MALDI TOF MS and fluorescence images of a section of a high-resolution array recorded from beads mixed with the solid state MALDI matrix.

FIGS. 42A-FIG. 42J are a series of MALDI TOF MS images of a high-resolution array produced from a bead library with ten distinct polypeptide analytes.

FIGS. 43A-43C presents an example of MALDI TOF-TOF mass spectrometry peptide sequencing performed on a microarray slide.

FIG. 44A and FIG. 44B present MALDI TOF MS spectra recorded on the MALDI target plate from a group of beads conjugated to a polypepitde and a large protein.

FIG. 45A and FIG. 45B present results of MALDI TOF MS scan of an analyte by two consecutive scans.

FIG. 46A, FIG. 46B, FIG. 46C, FIG. 46D, and FIG. 46E show examples of microarray image overlay.

FIG. 47A, FIG. 47B, FIG. 47C, and FIG. 47D show an example of using microarray image overlay (FIG. 47A, 47B) and scatter plot analysis (FIG. 47C, 47D) to establish interaction between two analytes.

FIG. 48A, FIG. 48B, and FIG. 48C show an example of visualization of microarray MSI data using a single mass channel and a continuous mass range.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

In an embodiment, there is provided a process that performs transfer of analytes from a library of microbeads onto a solid support. Such process enables: 1) simple and fast production of planar arrays containing a large number of analyte-containing microspots; 2) off-line analysis of bead-conjugated analytes by methods, such as mass-spectrometry, which require physical separation of samples from beads; 3) integration of optical detection with desorption-ionization MS and 4) integration of flow cell techniques with desorption-ionization MS.

This disclosure provides devices and methods that facilitate the use of mass spectrometry for the detection, characterization and quantitation of biological samples in bead-based multiplexed assays. The described process allows simultaneous transfer of samples from multiple beads onto the surface of a specially designed microarray plate. Some features of the presently disclosed embodiments are: (i) capability to handle bead libraries as large as 1,000,000 members on a single microarray chip, (ii) compatibility with a large variety of different bead assays and biological samples, (iii) the ability to transfer multiple samples from the same bead and (iv) facile interface with the industry-standard assay readout by fluorescence. The present disclosure eliminates the need for spotting robots in fabrication of large analyte arrays from libraries of microbeads. Furthermore, the presently disclosed embodiments facilitate application of the powerful technique of mass spectrometry imaging for the measurement of protein and other microarrays. This disclosure also provides methods that allow conversion of bead libraries into planar arrays.

The devices and methods disclosed herein provide means for achieving ultra compact arrangement of the analyte-containing spots on the solid support following the analytes elution from the beads, which enables analysis of bead libraries of large magnitude. The devices and methods of the present disclosure: 1) provide means to minimize the area of analyte-containing spots and ensure that the separation between adjacent spots is small, yet sufficient to prevent ambiguity in the assignment of analytes to a specific bead; 2) provide means to disrupt various linkages between the analyte and bead, while maintaining co-localization of various analytes eluted from the same bead; 3) provide means to elute analytes from beads using elution reagents that require incubation for an extended period of time to disrupt the analyte-bead linkages (e.g. digestive enzymes) while avoiding excessive migration of eluted analytes; 4) provide means to elute analytes from beads under conditions that enable quantitative detection of eluted analytes; and 5) provide means to perform bead- and solution-based biochemical reactions in microwells. The devices and methods of the present disclosure also enable the user to perform the above actions simultaneously for all members of the bead library without manual handling of individual beads and without the use of liquid dispensing or bead dispensing equipment. Additionally, devices and methods of the present disclosure utilize conditions that maximize the amount of analytes that are eluted from the beads and become accessible to the ionization beam of the mass spectrometer.

For example, the described process enables simultaneous transfer of analytes from libraries containing thousands to millions of individual beads that is performed under uniform conditions for all members of the bead library. It allows production of analyte-containing arrays with high spot density, at least 400 spots/mm², while maintaining reasonable spatial separation of the analytes eluted from different beads. The resulting microarray spots are compact, uniform and have high analyte density.

The described process further enables transfer of multiple analytes conjugated to individual beads by linkages of the same or different nature. For example the analytes may be conjugated to beads by covalent bonds, ionic bonds, hydrogen bonds, electrostatic interactions, hydrophilic interactions, hydrophobic interactions, or dipole-dipole interactions. More specifically, the analytes may be conjugated to beads by photo-labile bonds, acid-labile bonds, protease-sensitive bonds or antibody-antigen interactions. The analytes may be conjugated to beads directly or via another molecule or a group of molecules. The analytes may be also associated with beads by other means, for example they may be encapsulated or trapped in the interior of beads or other microparticles with or without forming specific chemical bonds with the bead material. A combination of elution strategies may be implemented for each bead library. The analytes eluted from individual beads remain co-localized within the same spot. Furthermore, the relative concentrations of various analytes conjugated to the same bead remain similar before and after the elution thus enabling a variety of applications that require analyte quantification.

The described process is compatible with numerous existing bead assay protocols including protocols that employ optical detection, in particular fluorescence and luminescence. The microwell array plates used as solid support are fully compatible with optical imaging using commercially available microarray scanners. Methods are described that use optical imaging of analytes on the microarray chips to distinguish between bead-bound and eluted analytes.

Additionally, the devices and methods of the present disclosure enable the user to selectively elute specific analytes from beads while retaining other analytes on beads, so that the bead material and the bead-bound analytes are not accessible to the ionization beam of the mass spectrometer. This is achieved by the unique design of the microwell plates, in which the microbeads are located in microwells below the surface spots containing their respective eluted analytes. While bead-bound analytes inside microwells are not detected by mass spectrometry, both eluted analytes and bead-bound analytes, as well as the beads themselves can be probed by optical methods, e.g. fluorescence.

The terms “elute,” “eluting” and “elution” that are used throughout this specification generally refer to the process of separating or releasing analytes from microbeads by various means, some of which are listed in FIG. 5.

The terms “array” and “microarray” refer to a group of analytes localized on a solid support in specific two-dimensional areas or in specific three-dimensional regions, which are referred to throughout this application as “analyte spots,” “microarray spots” or “spots.” In an embodiment, the term “microarray” refers to a group comprising a large number of distinct analyte spots, which are arranged on a solid support in a high-density framework. In one example, the microarray comprises at least 1,000 analyte spots and the area of a single microarray spot does not exceed 1 mm². The analytes localized on the solid support do not necessarily form a chemical bond with the solid support. The individual analyte spots may be spatially separated, or may exhibit some spatial overlap. One or several distinct analytes may be present in a single microarray spot. The analytes in analyte spots may exist in the solid state. In an embodiment, the analytes in analyte spots may exist in the liquid state, for example when mixed with a liquid MALDI ionization matrix. The microarrays referred to throughout this application are not necessarily reactive, i.e. they may or may not have the ability to bind and retain additional analytes. The microarrays of the present disclosure may contain additional chemical substances that facilitate the analyte detection, for example molecules of MALDI matrix for the microarray measurement by MALDI mass spectrometry. Such additional chemical substances may be present throughout the microarray or limited to the areas of analyte spots.

The term “analyte” refers to a substance or a chemical constituent that may be detected by an analytical method. For example, a molecule, a molecular fragment, a molecular complex, or singly or multiply ionized species may constitute an analyte. The term “analyte” may also refer to a plurality of identical species, e.g. identical molecules that are detected simultaneously by an analytical method.

The term “bead library” refers to a group of microbeads with one or several analytes bound to individual microbeads. In the context of this application, the term “microbead” may also refer to a microparticle that is not necessarily spherical.

The term “bead array” generally refers to a planar bead array, a group of microbeads spatially separated on a solid support in spatially addressable locations.

The term “small molecule” refers to an analyte that has a molecular weight of 1,000 Da or less.

The terms “microarray scan(ning) by MSI” and “microarray imaging by MSI” refer to the process of acquisition of mass spectrometric data from microarrays performed using methods of Mass Spectrometry Imaging, which is also known in the art as Imaging Mass Spectrometry.

The term “Mass Spectrometry Imaging” (MSI) refers to a mass-spectrometry based method of data acquisition, in which mass spectra are measured with the spatial resolving power of at least 1 mm.

The term “pixel” refers to a spatially addressable position within the microarray. For microarray images generated by the methods of MSI, a pixel may refer to a data point comprising a mass spectrum and coordinates of its location on the microarray.

The term “signal intensity” refers to a group of quantitative parameters including maximum peak intensity, mean peak intensity, area under peak, ion current and other mass spectral data that may be used to determine abundance or concentration of a specific analyte from its mass spectra.

The terms “global microarray analysis” and “global analysis” refer to the analysis of an area within the microarray dataset that comprises multiple pixels, up to the entire microarray area.

Throughout this specification, the use a singular form in descriptions, e.g. “molecule,” is intended to also include the plural form, e.g. “molecules” where appropriate. For example, a term “target molecule” may be used to describe multiple identical target molecules.

Methods are disclosed herein for converting a library of microbeads to a planar array of microspots that contain one or multiple analytes eluted from individual microbeads. In general, there is no restriction on the number of members in the bead library. In an embodiment, the libraries are between 100 and 500,000 members, however much larger bead libraries may be also accommodated on a single chip. In an embodiment, a single bead is capable of binding at least 10 femtomoles of analyte. The beads may be made of any suitable material, such as agarose including cross-linked agarose and other forms of chemically modified agarose, latex, polystyrene, polyacrylic gel, various other polymers, silica, glass, gel, or composite materials. Some of the bead types suitable for methods disclosed herein are sold under their respective trademarks, for example, TentaGel™ resins marketed by Rapp Polymere GmbH and Synbeads™ marketed by Iris Biotech GmbH. Various bead types, which are used in the field of solid-phase peptide synthesis including combinatorial solid-phase peptide synthesis, are also suitable for methods disclosed herein. The beads may be porous or non-porous and the analyte attachment to beads may be limited to the bead surface or occur also within the bead three-dimensional structure. The beads may have topologically segregated layers and the analytes bound to different layers within individual bead may require different conditions for elution. Individual beads may have distinctive optical properties, for example have distinctive absorption or transmission spectra in the UV, visible or IR range. Individual beads may also exhibit distinctive fluorescence or luminescence spectra. The beads may have magnetic properties, for example exhibit paramagnetic or superparamagnetic behavior. Furthermore, properties of the bead material may cause beads to swell upon exposure to a particular solvent. Beads of different size may be used. In an embodiment, the diameter of beads is between 1 and 1,000 micron. In an embodiment, the beads are spherical and monodisperse. Alternatively, the beads may have a size distribution within a specific range. In the latter case the difference in the diameter between any two beads within the bead library preferably does not differ by more than two-fold.

The described methods are compatible with many types of bead libraries and various types of analytes conjugated to beads. In an embodiment, the suitable beads already contain the analytes bound to the bead either directly, or by means of a linker molecule or a molecular complex. The linker molecules may constitute additional analytes if they become detached from beads following the elution. The present disclosure does not place a limit on the number of unique analytes attached to a single bead or the structure of linkers by which the analytes are attached to the bead. Methods of the present disclosure are not limited by the role of individual analytes in a particular bead assay. For example, the analytes may represent an affinity probe, affinity target, secondary probe, enzyme inhibitor, enzyme substrate, bead identification tag, etc. The analytes may also represent reagents used in the nano- and microparticle based drug delivery applications.

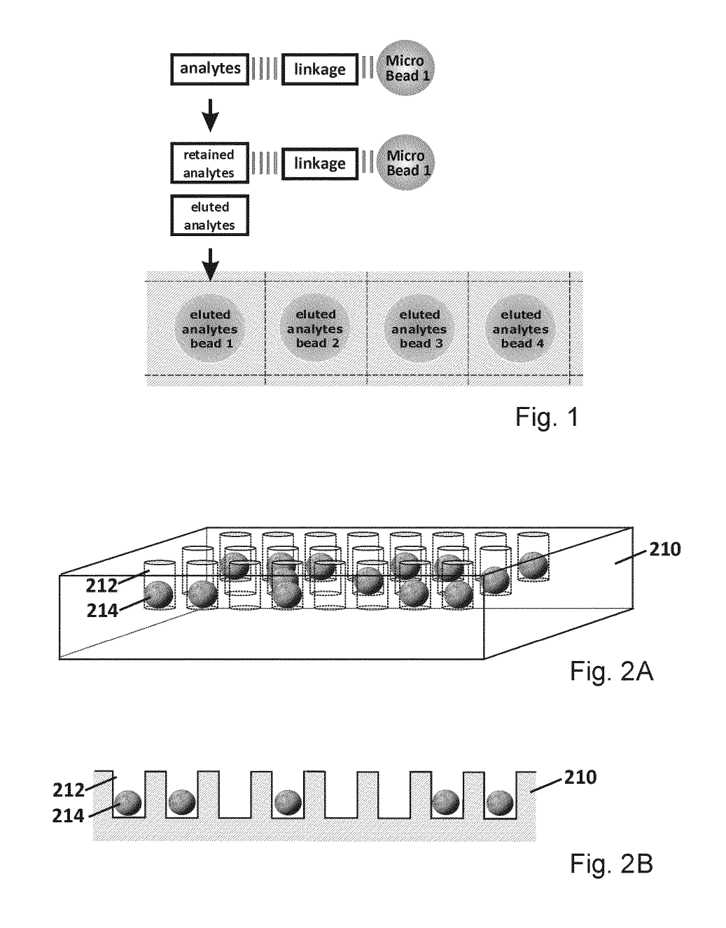

The ability to selectively transfer samples from microparticles onto a solid support is illustrated schematically in FIG. 1. In this depiction, one or several types of biomolecules collectively labeled as “analytes” are bound to an individual microparticle labeled “bead 1” by means of chemical bonds, intermolecular interactions, other molecules or molecular complexes collectively labeled as “linkage.” Methods of the present disclosure enable selective elution of specific molecules from each microparticle, which are collectively labeled “eluted analytes,” while leaving the remaining molecules bound to the bead, which are collectively labeled “retained analytes.” The analytes eluted from a single microparticle are localized in a single spot at the surface of the solid support, collectively labeled as “eluted analytes bead 1.” The described process is performed simultaneously for multiple microparticles resulting in fabrication of an array of microspots containing analytes eluted from individual microparticles. The fabricated microspots have similar size and shape and their linear dimensions are similar to the linear dimensions of their original microparticles. The microspots do not overlap with adjacent spots, or have limited overlap that does not preclude unambiguous identification of analytes within each spot.

Examples of analytes that can be transferred from microparticles include, but are not limited to, nucleic acids, small molecules with MW below 1,000 Da including small molecules of pharmaceutical importance, hormones, polypeptides, peptidomimetics, proteins including proteins with post-translational modifications, enzymes, antibodies, carbohydrates, lipids, antigens and their combinations. Furthermore, examples of analytes include larger structures comprising several molecules, such as protein-protein complexes, protein-carbohydrate complexes, protein-nucleic acid complexes and protein-lipid complexes. On the other hand, analytes may also comprise molecular fragments generated from molecules initially immobilized on beads, for example proteolytic fragments of a protein. In an embodiment, the analytes are compounds released from intact cells, which adhere to individual beads, for example by means of bead-conjugated cell surface binding ligands. The cells may represent bacterial, eukaryotic or mammalian cells. In an embodiment, the cells associated with individual beads represent a specific cell line or a specific cell type.

Solid Supports Suitable for Fabrication of an Array of Microspots from a Microbead Array

In an embodiment, microwell plates, which are sometimes referred to as picotiter plates, are used as the solid support for fabricating an array of microspots containing analytes eluted from individual microbeads. FIG. 2A and FIG. 2B show a schematic depiction of a microwell array plate 210 with several microwells 212 for accepting one or more microbeads 214 therein. In an embodiment, each microwell contains a single microbead.

In an embodiment, microwell array plates of the present disclosure are configured for extraction of analytes from individual microbeads and subsequent detection of analytes by mass spectrometry.

In an embodiment, the microwell array plates of the present disclosure are different from the mass spectrometry-compatible devices known in the prior art including MALDI target plates, surface enhanced target plates, individual microvials, microvial and nanovial arrays and surfaces capable of binding microbeads. In an embodiment, the microwell array plates of the present disclosure are configured to retain individual bead, provide spatial separation for individual bead, or both. In an embodiment, the microwell array plates of the present disclosure are configured to allow efficient elution of analytes from individual bead. In an embodiment, the microwell array plates of the present disclosure are configured such that liquid dispensing equipment is not required in order to use the microwell array plates of the present disclosure. In an embodiment, the microwell array plates of the present disclosure are configured such that liquid dispensing equipment, for example a robotic matrix spotter, may be used in conjunction with the microwell array plates of the present disclosure.

In an embodiment, the microwell array plates of the present disclosure are designed for analysis of individual microbeads. In an embodiment, the microwell array plates of the present disclosure are configured to separate beads into individual microwells. In an embodiment, the microwell array plates of the present disclosure are configured to be used without liquid handling robots or manual pipetting for bead and solution dispensing. In an embodiment, the microwell array plates of the present disclosure are configured to be used without additional equipment to generate external pressure or vacuum necessary to perform the sample washing and elution steps. In an embodiment, the microwell array plates of the present disclosure are configured to provide compatibility with the optical detection of beads, the optical detection of analytes on beads, the optical detection of analytes eluted from beads, or all of the above.

In an embodiment, the microwell array plates of the present disclosure are configured to retain individual beads without forming a linkage, i.e. a chemical bond between the microwell plate and individual beads. In an embodiment, the microwell array plates of the present disclosure are configured to be used without mechanical devices such as pins to transfer individual beads into the individual microwells. In an embodiment, the microwell array plates of the present disclosure are configured such that the individual beads can be distributed into wells without using bead sorting equipment and bead dispensing equipment. In an embodiment, the microwell array plates of the present disclosure are configured to allow desorption of analytes eluted from beads to occur from the surface-proximal layer.

In an embodiment, the microwell array plates of the present disclosure are configured to enable analysis of bead arrays by methods of desorption-ionization mass spectrometry. Unlike mass spectrometry, optical detection methods do not require physical separation of analytes from beads. To that end, in an embodiment, the microwell array plates of the present disclosure are configured to enable physical separation of analytes from beads. By way of a non-limiting example, specific parameters that enable the use of microwell array plates of the present disclosure in mass spectrometric applications include, but are not limited, to (1) geometry of the plates; (2) surface properties and (3) optical properties.

In an embodiment, the microwell array plates of the present disclosure are configured to perform MS analysis of individual beads. In an embodiment, the microwell array plates of the present disclosure are configured to enable multiple bead analysis by mass spectrometry while avoiding manual selection and deposition of individual beads on the MALDI target plate and manual analyte elution from beads. In an embodiment, the microwell array plates of the present disclosure are configured to restrict analyte migration. In an embodiment, analyte migration is restricted to an area comparable to the area occupied by a single microbead. In an embodiment, the microwell array plates of the present disclosure are configured to control the size of analyte spots and to prevent formation of very large spots, that is spots having a diameter substantially greater than the diameter of its parent microbead. In an embodiment, the microwell array plates of the present disclosure are configured to localize eluted analytes in order to prevent or at least minimize dilution of analyte concentration. In this manner, the microwell array plates of the present disclosure may accommodate a large number of microbeads on a single chip.

In an embodiment, microwell array plates of the present disclosure comprise a block of solid material containing a plurality of microwells, pits, depressions or similar features. The microwell plates may have the shape of a rectangular prism or have a similar shape. In an embodiment, linear dimensions of the microwell plate are approximately 75 mm×25 mm×1 mm, measured as length×width×height. The microwells may have the shape of a cylinder, inverted cone, inverted pyramid, rectangular prism or other shape.

Microwell plates may be made of various materials including metals, such as stainless steel, polymers, various types of glass and silicon. The microwell array plates of the present disclosure may be manufactured using various techniques known in the art, for example, soft lithography, photolithography, injection molding, acid etching and laser ablation. In an embodiment, microwell array plates are manufactured from fiber optic bundles.

A small section of an exemplary microwell array plate 210 is shown in FIG. 2A and FIG. 2B. In this example, each of the microwells 212 is 42 micron in diameter and 55 micron deep with the 50 micron distance between the centers of adjacent wells. The microwells serve to retain microbeads 214 within the plate and provide spatial separation between individual beads. The diameter of a microwell is selected to be slightly larger than the diameter of a microbead, which ensures that no more than one bead can occupy a single microwell. The distance between individual microwells, which controls separation between analyte spots, may vary depending on a specific application. The microwells are preferably arranged in a specific order, for example a square, rectangular or hexagonal grid to facilitate subsequent application of MALDI matrix, MS measurement and analysis of the fabricated analyte arrays. The depth of microwells relative to the bead diameter may vary, as shown in specific examples below.

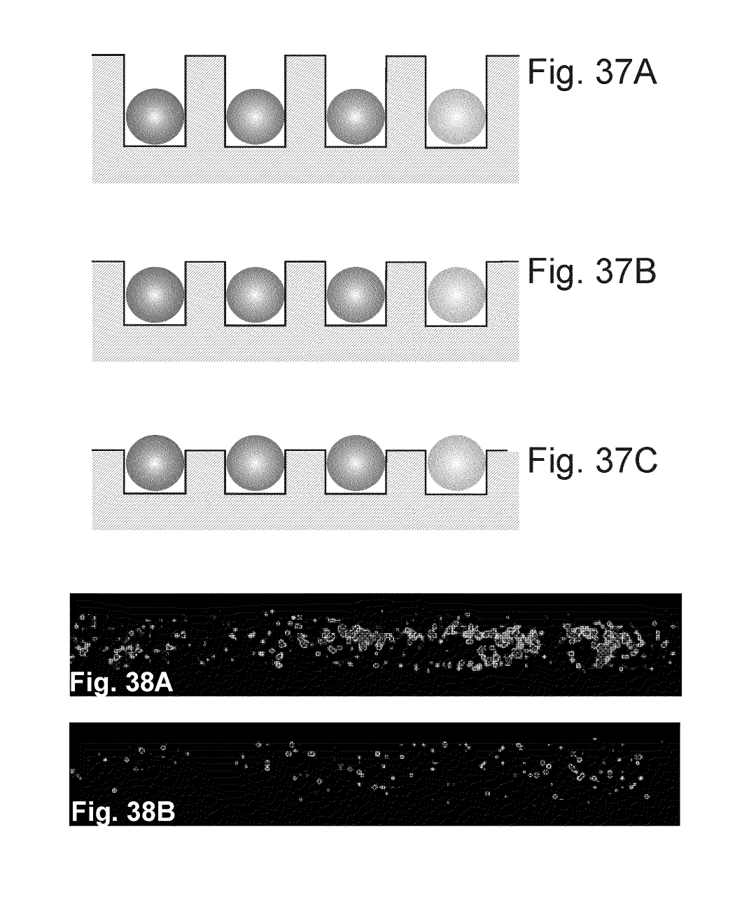

In an embodiment, in the microwell plates of the present disclosure, microwells are provided with a specific depth, which is determined on the basis of utilized methods of analyte elution from beads, methods of MALDI matrix application and/or the type of ionization matrix. As described, for example, in Example 13 and Example 14 and is shown in FIG. 37, the position of beads relative to the plate surface, which is a function of the microwell depth and the bead diameter, may impact the efficiency of analyte elution from beads. In general, placing microbeads close to the surface may allow more efficient analyte elution and greater accessibility of eluted analytes to the ionization beam of the mass spectrometer. However, in an alternative embodiment disclosed in Example 16 and Example 17, microbeads are placed at a greater distance from the surface to enable the use of solid phase MALDI matrix for analyte ionization. In an embodiment, microbeads are 34 micron in diameter and the depth of microwells is in the 30-55 micron range. A range of suitable depths is provided below. In an embodiment, both the diameter of microbeads and the depth of microwells are variable parameters to allow for customization of the beads and microwell plates. In an embodiment, the desired depth of microwells is expressed as a fraction of or a multiple of the bead diameter. In an embodiment, the microwells have a minimum depth sufficient to retain the beads in fixed positions on the plate and the maximum depth sufficient to allow elution and detection of analytes eluted from a single bead placed inside the well. In an embodiment, the range of suitable microwell depths for a library containing microbeads of a specific diameter is between ½ of the bead diameter and 2-fold the bead diameter. For example, for 34 μm beads, the preferred minimum well depth is 17 micron and the preferred maximum well depth is 68 micron. Note that it is also possible to provide much larger depths, e.g. 5-fold of the bead diameter or even greater, which is still within the thickness of the microwell array plate. In an embodiment, the microwells are sized to accommodate a single bead. This can be accomplished for example, by loading microbeads at sufficiently low density. Methods for estimating the depth and profile of microwells are known in the art.

In an embodiment, in the microwell plates of the present disclosure, microwells have a uniform depth. Providing microwell plates with microwells of uniform depth may ensure the identical position of the beads inside their respective microwells. This, in turn, may ensure similar conditions for the analyte transfer from the beads onto the microarray plate. In an embodiment, the microwell plates of the present disclosure with this feature are used for applications in the field of quantitative proteomics. In an embodiment, the depths of any two microwells within the microwell plate preferably differ by less than 10%, more preferably by less than 5%, most preferably by less than 1%.

In an embodiment, in the microwell plates of the present disclosure, there is provided a specific distance between the centers of adjacent microwells. In an embodiment, the larger spacing between individual wells may benefit applications that require analyte elution from the beads, as the means to reduce the spot overlap. In an embodiment, individual microwells are 42 micron in diameter and the distance between centers of adjacent microwells is 50 micron. A range of suitable distances is provided below. Note that: (1) the diameter of microbeads, (2) the diameter of microwells and (3) the distance between the centers of adjacent microwells are variable parameters such that the microwell plates and beads can be customized for a particular application. Therefore the desired distance between the centers of microwells can be expressed as a multiple of the microwell diameter. In an embodiment, the average distance between the centers of adjacent microwells is not less than 1.2-fold of the well diameter and not more than 10-fold of the well diameter. For example, for wells that are 42 micron in diameter, the minimum separation distance is approximately 50 micron and the maximum separation distance is 420 micron. The increase in the separation distance between individual microwells proportionally increases the surface area that is not occupied by openings into the microwells. This area may accommodate analytes that “spill over” from individual microwells during the elution from microbeads. Methods for estimating separation between individual microwells, e.g. by scanning electron microscopy, have been described in (Pantano and Walt Chemistry of Materials 1996).

In an embodiment, the diameter of microwells can be expressed as a multiple of the bead diameter. In an embodiment, the minimum well diameter is equal to 1.1-fold of the bead diameter and the maximum well diameter is equal to 2-fold of the bead diameter. For example, for 34 micron beads, the minimum diameter is 38 micron and the maximum diameter is 68 micron. It is also possible to provide microwells with even larger well diameter, however such larger wells will be able to accommodate more than one bead per well. On the other hand, microwell plates featuring wider microwells may be provided to accommodate microbeads that swell upon exposure to a particular solvent. Methods for estimating the diameter of individual microwells, e.g. by scanning electron microscopy, have been described in (Pantano and Walt Chemistry of Materials 1996).

In an embodiment, the microwell plates of the present disclosure are provided with the microwells arranged in a highly precise regular grid. The MALDI MS measurements are usually performed by providing the instrument with a specific scan pattern, i.e. providing exact coordinates of the first spot to be measured, as well as coordinates of the subsequent spots. Accordingly, in the microwell plates of the present disclosure, the microwells are disposed in an ordered arrangement such that each MS spectrum may be acquired near the center of microwells where the analyte concentration is the highest. Such arrangement may also help to eliminate ambiguity in the assignment of analytes to a specific bead/microwell. The ordered arrangement of microwells may also facilitate the use of liquid dispensing robots to apply MALDI matrix solution in locations that coincide with the positions of microwells. In an embodiment, the microwells on the plates are positioned so that the centers of wells form a specific pattern, for example a hexagonal or square grid. In an embodiment, there are no missing wells within such grid. In an embodiment, the centers of microwells within each row and column form a straight line. In an embodiment, a displacement of the center of an individual well from such straight line is less than ½ of the well diameter. In an embodiment, a displacement of the center of an individual well from such straight line is less than ¼ of the well diameter.

In an embodiment, the surface of microwell plates of the present disclosure is provided with a surface layer, comprising a hydrophobic, non-reactive, electrically conductive and optically transparent material. An example of material that satisfies the above requirements is a conductive transparent oxide, for example Indium Oxide or Indium Tin Oxide (ITO). Another example of material that satisfies the above requirements is Gold. Although Gold has limited transparency in the visible range, a thin layer of this material, for example between 1 and 10 nm, is sufficiently transparent to enable detection by optical methods. Other materials may also be used. Suitable methods of depositing a thin film on a solid substrate include, but are not limited to, electron beam evaporation, physical vapor deposition, sputter deposition or similar.

In an embodiment, by providing the surface layer as described above on the surface of microwell plates may serve to: (i) achieve better localization of eluted analytes; (ii) ensure stability of eluted analytes on the solid support and (iii) perform more accurate measurement of eluted analytes by mass-spectrometry and optionally also by optical detection. The hydrophobic coating may prevent migration of eluted analytes on the surface of a microwell plate and retains the eluted analytes in the vicinity of microwells. In an embodiment, the combination of a hydrophobic surface and an array of microwells of the present disclosure provides localization of analytes eluted from the microwells. In an embodiment, the combination of a hydrophobic surface and an array of microwells effectively creates a pattern of alternating hydrophobic and hydrophilic areas, in which hydrophilic areas coincide with openings into the microwells. Microbeads placed inside microwells, which are within a short distance from the surface, may further contribute to the hydrophilic character of these areas. As a result, an aqueous solution uniformly applied as an aerosol to a microwell plate modified with a hydrophobic surface layer may accumulate in discrete droplets in the hydrophilic areas within the openings into the microwells, thus improving contact between the microbeads and the aqueous solution.

Modification of the solid support with material that is chemically non-reactive may enable off-line analysis, storage and archiving of fabricated arrays of analytes while reducing the risk of analyte degradation due to its interaction with the solid support. Furthermore, in an embodiment, Gold or another material with similar relevant properties may be suitable for surface coating because of its weak interaction with biomolecules and MALDI matrices. The absence of strong interaction (e.g., adsorption) between the material of solid support and the analyte-MALDI matrix mixture may facilitate subsequent desorption—ionization of eluted analytes. In an embodiment, a surface layer is coated on the surface of microwell plates for analytes in the higher molecular weight range, such as for example, above 2,000 Da,

Fabrication of an Array of Microspots from a Bead Array

Prior to forming a bead array, bead libraries may be stored in any suitable medium, which is compatible with the bead chemistry and ensures stability of the analyte molecules attached to beads. The specific non-limiting example of a suitable medium is deionized water. More generally, any common biocompatible medium may be used, including solutions containing various additives such as glycerol, salts, buffers, detergents, bacterial growth inhibitors, proteolysis inhibitors etc. In an embodiment, the additives are removed by incubating beads in deionized water prior to the bead loading on the microwell array plate. The bead libraries may be stored under conditions that ensure stability of beads and the analyte molecules attached to beads. For example, beads can be refrigerated and protected from light.

Microbeads used for the analyte transfer may be supplied in contaminant-free medium. Examples of contaminants are glycerol, salts, detergents, buffers or other similar chemicals, which may interfere with the subsequent detection of analytes by mass spectrometry or other methods. However, trace amounts of contaminants may remain as long as they do not adversely affect the performance of analytical methods used to measure the resulting microarray. The removal of contaminants is achieved by replacing the original medium, in which the suspension of beads is supplied, with a desired medium. The desired medium may be pure deionized water or contain additives to enhance the assay performance. The examples of additives include Dithiothreitol (DTT) and Tris(2-carboxyethyl)phosphine (TCEP), oxidation inhibitors, or slow evaporating solvents. If needed, the medium exchange process may be repeated several times, until the desired degree of purity is achieved. More specific washing procedures have been described in protocols available for various bead assays.

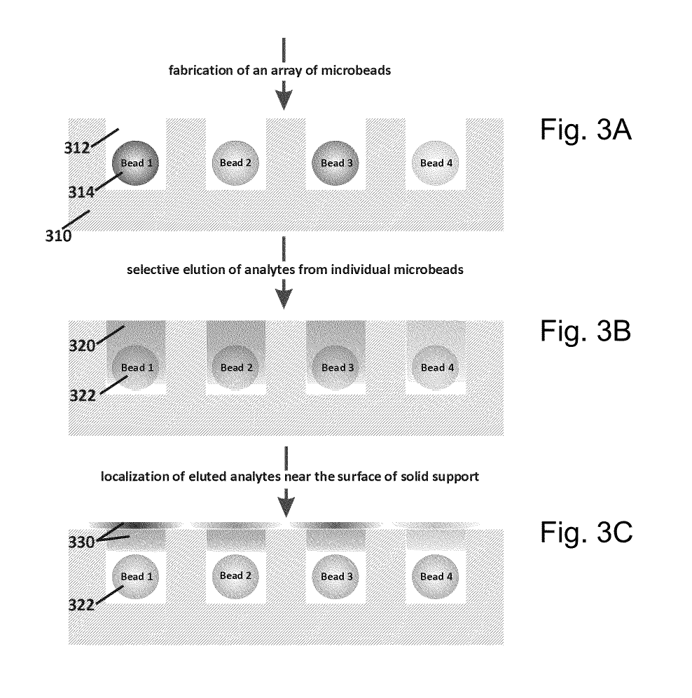

FIG. 3A, FIG. 3B, and FIG. 3C schematically illustrate an embodiment of a method of transferring analytes from a bead library onto a solid support, such as a microwell array plate. Such method generally comprises the steps of: 1) applying a suspension of microbeads onto a microwell plate and spatially separating individual microbeads i.e. fabricating an array of microbeads (FIG. 3A), 2) eluting analytes from beads and retaining the eluted analytes in the vicinity of their respective microbeads (FIG. 3B), and 3) localizing the eluted analytes at the surface of the microarray plate in the form of discrete spots (FIG. 3C). FIG. 3A, FIG.3B, and FIG. 3C show a side view of a section of an embodiment of a microwell array plate 310 for each of the three steps. During the step shown in FIG. 3A, beads with bound analytes 314 are loaded into individual wells 312 preformed on the surface of a microwell array plate 310. In an embodiment, each microwell 312 contains no more than one microbead. Multiple distinct analytes may be bound to individual microbeads. Methods of the present disclosure enable elution of one or several analytes from individual beads. During the step shown in FIG. 3B, the analytes are eluted from the microbeads placed inside individual microwells 312. The elution procedure may comprise several reactions that are performed concurrently or consecutively. Analytes 320 eluted from each bead 322 preferably remain within the corresponding microwell 312, i.e. their migration on the microwell plate is limited to a vicinity of their respective beads. During the step shown in FIG. 3C, the eluted analytes 330 become localized in discrete spots near the surface of the microwell array plate 310. In the case of multiple analytes eluted from a single bead, the eluted analytes are co-localized within the same spot. Each of the steps 1 through 3 may be performed simultaneously for all members of the bead library.

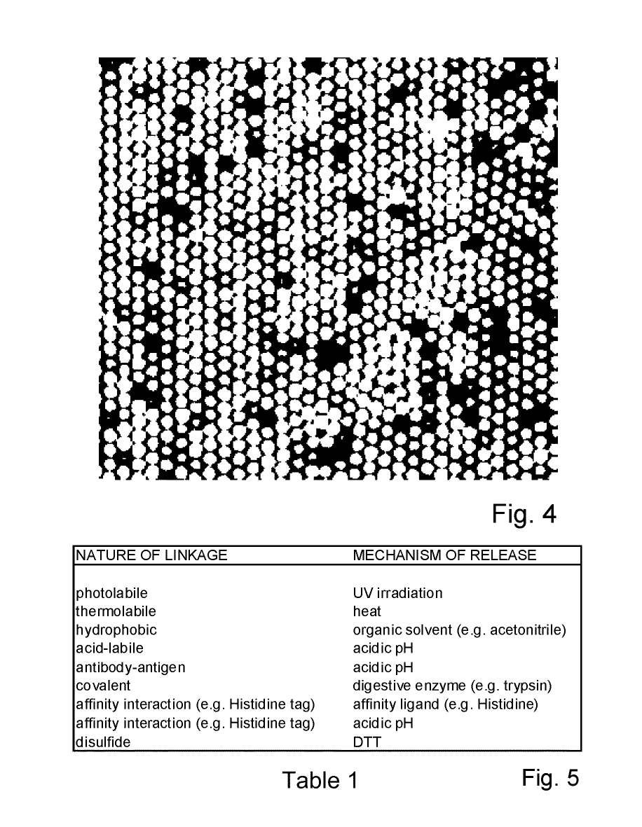

In an embodiment, the first step of the process according to FIG. 3A is loading of the bead library onto the solid support. In an embodiment, the beads are placed inside pre-fabricated microwells arranged in a regular grid on the microwell array plate. An image of a small section of an exemplary microwell array plate is shown in FIG. 4. In this example, each of the microwells is 42 micron in diameter and 55 micron deep with 50 micron distance between the centers of adjacent wells. The microwells serve to retain beads on the plate and provide spatial separation between individual beads. The diameter of microwells is selected to be slightly large than the bead diameter, thus ensuring that no more than one bead can occupy a single microwell. The distance between individual microwells serves to control the spot separation. The microwells are preferably arranged in a specific order, for example a square grid or hexagonal grid, to facilitate subsequent imaging and analysis of the fabricated analyte array. The depth of microwells relative to the bead diameter may vary, as shown in specific examples below.

The beads may be supplied as a suspension in deionized water or other suitable medium, applied to the surface of a microwell array plate, settle into individual microwells by gravity and moved to the bottom of microwells by centrifugation. The beads, which are loaded into the microwells, essentially become immobilized on the microwell array plate. The ability to retain microbeads in individual wells may enable the use of microwell array plates with immobilized beads as flow cell devices as disclosed in detail below. Accordingly, in an embodiment, the microbeads are held in place without having to form a chemical bond with the solid support. Loose beads that remain on the surface of microwell plate after centrifugation may be removed by rinsing the plate with deionized water or other suitable medium. In an embodiment, beads placed inside the microwells are kept hydrated until the analytes are eluted. In an embodiment, additional reagents are loaded into the microwells that already contain the beads. For example, solid phase microcrystals of MALDI matrix can be loaded inside the microwells as shown in the examples below.

The device and methods described in Step 1 (FIG. 3A) enable fabrication of a random bead array, which can be used to perform selective elution of one or multiple analytes from individual beads and localization of the eluted analytes near their respective microbeads. In an embodiment, such random bead arrays are self-assembled, and thus do not require additional dispensing equipment, e.g. bead dispensing robots or liquid dispensing robots.

The bead arrays fabricated in Step 1 (FIG. 3A) may deviate from an ideal array pattern, such as a hexagonal or square grid. For example, some microwells may remain empty, i.e. not occupied by beads. This may occur, for example, if the total number of beads loaded on the microwell plate is smaller than the total number of wells. In that case, the distribution of beads on the plate may be uniform, or may comprise areas with greater concentration of beads and areas with lower concentration, as well as areas that are not occupied by beads. Also, it should be understood that in some embodiments of the disclosed experimental procedures microwells may be occupied by two or more different beads. In an embodiment, the appearance of microwells with two or more beads is limited to a small fraction of the total microwell plate capacity, preferably below 5% and more preferably below 1%.

Bead arrays fabricated according to the disclosed methods may be stored for an extended period of time under appropriate conditions. For example, bead arrays may need to be chilled or refrigerated, protected from light and stored in a humidified environment. The fabricated bead arrays may be also prepared on-site and shipped to a different location using precautions normally associated with shipping perishable materials.

In an embodiment, the second step of the process, as shown in FIG. 3B, is elution of the analytes from microbeads. This step includes selection of an appropriate elution method or a group of elution methods, which is determined by several factors. First, the elution method may be determined based on the structure of the analyte-bead linkers. Second, the elution method may be selected to release only the analytes, which will be subsequently measured by mass spectrometry and other analytical methods, i.e. compounds, which are not intended to be measured by mass spectrometry, should remain conjugated to the beads. The elution method may be specific for each bead library. The exemplary list of different linkages and appropriate elution mechanisms is listed in Table 1, shown in FIG. 5. For example, exposure of beads arrayed on the plate to the light of specific wavelength or specific wavelength range is selected for the release of analytes conjugated to beads by a photolabile linker, which is photosensitive to the specific wavelength. Common photolabile linkers are photosensitive to the long wavelength UV light. Alternatively application of a low-pH solution to beads arrayed on the plate is used for the release of analytes conjugated to beads by an acid-labile linker such as the antibody-antigen interaction. Alternatively, application of a solution containing acetonitrile is selected for the release of analytes conjugated to beads by hydrophobic interactions. Other examples of elution methods include: heat, application of a digestive compound and application of a ligand with the similar affinity for the binding sites as the analyte (i.e. competitive elution). Various methods of releasing analytes from a bead are known in the art and may be employed in the methods of the present disclosure.

For the elution of multiple analytes from beads, either a single elution method or a combination of elution methods may be required. For example, multiple analytes bound to beads through the acid-labile antibody-antigen interactions may be eluted simply by exposure to the acidic medium. When a combination of elution methods is required, the elution may be performed either concurrently or consecutively. The example of a concurrent elution is application of the MALDI matrix solution, which contains both an acid (TFA) and organic solvent (acetonitrile). The example of consecutive elution is irradiation with UV light followed by incubation with a digestive enzyme.

The elution reagents can be delivered to the beads in the solid, liquid or gas form. In an embodiment, the method of delivery for elution reagents maintains spatial separation between analyte spots formed from individual microbeads. For example, the liquid reagents may be delivered in droplets with the size of droplets being considerably smaller than the diameter of individual microbeads. Such droplets can be generated by a variety of instruments including airbrushes, nebulizers, TLC sprayers or MALDI matrix spotting robots. In an embodiment, the droplets are not allowed to merge into much larger spots on the chip, i.e. the microwell plate, that would cover the area containing multiple microbeads. This may be accomplished, for example, by limiting the amount of solution delivered to the chip, for example by selecting the duration of solution application. This may also be accomplished by evaporation of excess solution from the plate. Furthermore the elution reagent can be delivered in multiple cycles with specific amount of time allowed for incubation of the beads with the elution reagent to ensure optimal analyte release. Using the above procedures allows the analyte molecules that are released from individual beads to remain in the vicinity of their original microbeads on the microwell array plate.

In an embodiment, the migration of eluted analytes is limited to the vicinity of individual microbeads and therefore allows formation of very compact microarray spots. In an embodiment, this is accomplished by reducing the amount of bulk liquid on the surface of a microarray plate and using hydrophobic solid support, as described in detail in this specification. Detailed protocols for the delivery of liquid reagents to the slide are also described in the MATERIALS AND METHODS. In an embodiment, linear dimensions of microarray spots formed on the surface of the solid support after the analyte elution from beads of specific diameter are between 1-fold and 3-fold of the diameter of microbeads. For example, for 34 micron microbeads, the diameter of analyte spots formed on the microarray is between approximately 34 micron and approximately 100 micron. In an embodiment, the dimensions of analyte spots on the microarray are not greater than the dimensions of individual microwells, which contain the microbeads. For example, for 34 micron microbeads placed inside 42 micron microwells, the diameter of spots formed by analyte elution from such microbeads is not larger than approximately 42 micron. Non-limiting experimental methods that restrict the analyte migration to individual microwells are described in detail in Example 16 and Example 17, among others.

In an embodiment, the elution methods of the present disclosure result in co-localization of different analytes eluted from the same microbead, including co-localization of fluorescent and non-fluorescent analytes. In an embodiment, the elution methods of the present disclosure result in co-localization of the analytes eluted from the same bead with an optional fluorescent label, which remains immobilized on bead. The conditions that aid in co-localization of different analytes include, but are not limited to, methods described in the previous paragraph, namely spatially limited migration of the analytes on the microarray plate after their elution from the beads. This is achieved by preventing formation of large droplets of liquid medium on the surface of the plate. In an embodiment, the microarray plate area is uniformly coated with the small droplets (e.g. aerosol or mist) of the liquid medium, which contains the elution reagent. The size of the droplets formed on the surface of the slide may be determined by the following parameters: (1) properties of the device used to generate said aerosol or mist, (2) the experimental protocol of the delivery of said aerosol or mist to the plate and (3) surface chemistry of the plate. The Experimental Examples shown in this disclosure were obtained with the 3.5 micron aerosol particles (mass median diameter) generated by the PARI LC Sprint reusable nebulizer. In an embodiment, the mass median diameter of aerosol particles delivered to the plate is between 0.03 and 0.3 of the diameter of microbeads. For example, for 34 micron microbeads the mass median diameter of aerosol particles is between 1.0 and 10 micron. The experimental protocols of aerosol delivery to the slide and description of the microwell array plates are given in the MATERIALS AND METHODS section. The uniform coating of the microarray plate area with small droplets containing the elution reagent also results in the uniform pattern of analyte elution across the entire chip as shown in Example 9.

The present disclosure also provides conditions that achieve quantitative co-elution of different analytes from the same bead. These conditions may include, but are not limited to, (1) elution of a substantial fraction of each analyte from the beads and (2) providing specific amount of time to allow for diffusion of eluted analytes. With respect to the first condition, preferably between 5% and 100% of the total amount of analyte is eluted from individual beads, more preferably between 25% and 100%. With respect to the second condition, preferably between 30 sec and 5 mins is allowed for the analyte diffusion before the solvent is removed.

In the methods of analyte elution of the present disclosure, the known pattern of beads arranged on the microwell plate, which is determined by the grid of microwells, may be used by matrix spotting robots to dispense ionization matrix-containing solution precisely in positions matching the locations of microwells. In contrast, such approach is not possible in the tissue imaging applications where the analyte distribution is continuous rather than discrete. Also, the available information about the composition and properties of the microbeads, analytes, and the analyte-bead linkages may be used to provide an elution protocol, in which the chemical composition of elution reagents and the sequence of experimental steps are optimized for a specific bead library including bead libraries with different types of analyte-bead linkages. This approach can be beneficial in programmable liquid dispensing devices that are used to automate the elution and matrix applications steps.

In an embodiment, the third step of the process, as shown in FIG. 3C, is localization of the eluted analytes on the microarray plate in the form of discrete spots. In an embodiment, this is achieved by removing the liquid medium (solvent) from the solid support. In an embodiment, following their elution from beads in step two the analyte molecules are not immobilized on the microarray plate. Rather, the analyte molecules may remain dissolved or suspended in the liquid medium and are able to diffuse in the vicinity of their respective beads. The diffusion may serve to enhance the analyte extraction from the beads. The diffusion may be desired in the case of complex multi-component constructs immobilized on beads, which may include both high and low molecular weight analytes, such as full-length proteins and short polypeptides as shown in Example 12. Providing specific length of time to allow for the analyte diffusion in the vicinity of their respective microbeads serves to enhance the analyte extraction from beads. In an embodiment, between 30 sec and 1 min are provided after the analyte elution and before the solvent removal. In an alternative embodiment, between 1 min and 10 min are provided after the analyte elution and before the solvent removal. In an embodiment, between 30 min and 6 hours are provided after the analyte elution and before the solvent removal. During this time, the analyte migration can be restricted to a specific area of the microwell plate by any one or more of the following: (i) performing elution reaction entirely inside individual microwells, which provide spatial separation for analytes eluted from different microbeads; (ii) limiting the amount of liquid medium on the solid support and the duration of bead exposure to the liquid medium; (iii) selecting viscosity and hydrophobicity of the elution solvent, in combination with the surface properties of the solid support, that will allow formation of discrete spots as opposed to excessive migration of the analytes on the surface and (iv) using solid phase matrix to minimize the analyte migration. Specific protocols are given in the examples below.

The process of fabricating spots of analytes involves removing the liquid medium from the solid support after the specific amount of time allowed for diffusion, i.e. the analytes are allowed to dry and thus become immobilized on the solid support. Removal of the liquid medium may be achieved by evaporation and may serve to: (1) enhance the migration of eluted analytes from microbeads toward the surface of the microwell plate; (2) localize eluted analytes in specific areas of the microwell plate, e.g. directly above their respective microbeads and prevent their further migration on the microwell plate and (3) immobilize eluted analytes at the surface of the microwell plate in the form that allows their subsequent desorption-ionization for mass spectrometric analysis. Either air-drying or vacuum drying, among other similar methods, can be employed. In an embodiment, vacuum drying is employed if a slow evaporating solvent is present. If the fabricated microarray is subsequently to be measured by MALDI mass spectrometry the MALDI matrix solution is applied to the beads as described in more detail in Step 2, preferably before the solvent is completely removed. As described above, the application of MALDI matrix solution can be used both to elute analytes from beads, and to facilitate ionization of the analyte molecules by mixing them with the matrix molecules. As a result of the process described in steps one through three an array of spots containing concentrated analytes may be produced, which can be measured by desired analytical methods but also archived and stored for off-line analysis.

In an embodiment, the method for immobilization of analytes at a surface of a microarray plate and their subsequent analysis by mass spectrometry of the present disclosure may result in one or more of the following: (1) fabrication of two arrays, i.e. an array of microbeads and a separate array of microspots on the same solid support; (2) limiting migration of the released analytes, so that the dimensions of individual microspots are similar to the dimensions of individual microbeads and (3) providing separate steps for the analyte release from the microbeads and the analyte localization in microspots, which may be useful, for example, when complex analyte compositions comprising diverse molecules are present. The disclosure and Experimental Examples are written using the example of MALDI MS. Previously, spray deposition of the MALDI matrix solution on thin slices of tissue has been described for the tissue imaging by MALDI mass spectrometry. In some methods of the present disclosure, this technique is adapted for the fabrication of arrays of microspots containing analytes eluted from individual microbeads. Although the MALDI technique is used as an example throughout this specification, numerous alternative mechanisms of analyte ionization, ionization matrices and techniques of matrix application to the analytes may be implemented that are within the scope of the present disclosure. For example, liquid matrices including ionic liquid matrices that are suitable for IR or UV MALDI (Tholey et al. Anal Bioanal Chem 2006) or matrices suitable for liquid SIMS may be loaded inside microwells either prior to or subsequently to the microbeads, mixed with analytes eluted from microbeads and used for the analyte ionization. Microwell array plates provide physical separation between individual microwells filled with the analyte-matrix solution and therefore are ideally suited for high-lateral resolution MS analysis using liquid ionization matrices. In this implementation, although the liquid matrix occupies the entire volume of a microwell, only the surface-proximal layer is accessible to the ionization beam of the mass spectrometer and thus represents an analyte spot.

In an embodiment, nanoparticles either unmodified or functionalized with specific ligands may be used for desorption of analytes eluted from microbeads inside individual microwells. Nanoparticles may be loaded into individual microwells on top of the analyte-conjugated microbeads by gravity, centrifugation or application of magnetic field. Various other techniques of matrix delivery to the solid support including for example, methods of sublimation-deposition, which are all within the scope of the present disclosure, will be apparent to a person skilled in the art.

A non-limiting example of experimental procedure that uses microcrystals of MALDI matrix to fabricate an array of microspots from a bead array is disclosed below. This method enables downstream analysis of bead arrays by MALDI MS but is significantly different from the previously disclosed methods that involve spray deposition of MALDI matrix solution. The techniques disclosed below are also applicable to the fabrication of an array of microspots using nanoparticles for nanoparticle-based mass spectrometry.

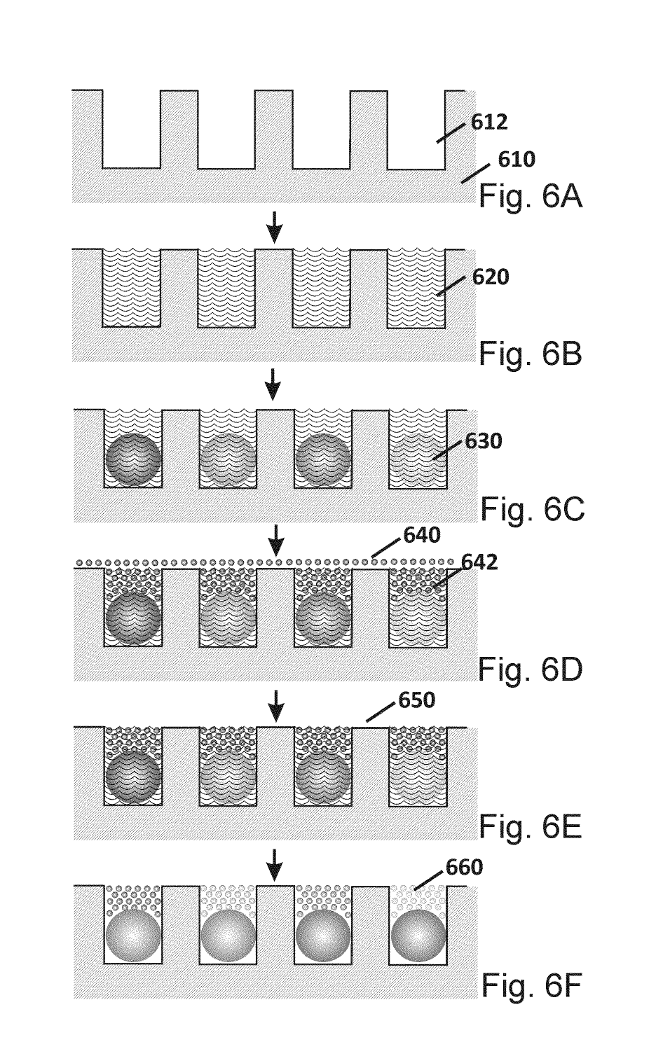

Schematic representation of an embodiment method of the present disclosure is shown in FIG. 6A through FIG. 6F. In reference to FIG. 6A, a cross-section of a small part of a microwell array plate 610 depicts a group of microwells 612. The microwells may be filled with a liquid medium 620, such as deionized H₂O prior to loading microbeads into microwells, as shown in FIG. 6B. The microbeads 630 are loaded into microwells filled with the liquid medium 620 using previously disclosed methods, for example by centrifugation, as shown in FIG. 6C. A specific distance is provided between the surface of a microbead and the surface of the microwell array plate, which is determined by the difference between the depth of microwells 612 and the diameter of microbeads 630. In an embodiment, the distance is greater than 0.1 of the microbead diameter and smaller than 0.95 of the microbead diameter. In an alternative embodiment, the distance is smaller than 0.1 of the microbead diameter, e.g. the beads are very close to the surface of the microwell plate.

Next solid phase microcrystals of MALDI matrix layer 640 is deposited on the surface of the microwell plate, as shown in FIG. 6D. Solid phase microcrystals of MALDI matrix may be prepared by various methods known in the art. For example, matrix microcrystals may be prepared by grinding larger crystals and filtering the ground crystals through a sieve to obtain microcrystals of specific size or size distribution. In an embodiment, the microcrystals are between 0.1 and 20 micron, preferably between 0.3 and 3 micron. Examples of MALDI matrices that can be prepared using this technique include CHCA, SA and DHB.

The solid microcrystals of MALDI matrix are deposited on the microwell plate using gravity and optionally centrifugation, which is performed after loading the microbeads on the same microwell plate. The matrix crystals fill the microwell space 642 that is not occupied by the bead and also form a matrix layer 640 on the surface of the microwell plate. The plate may be optionally rinsed with deionized water or other suitable medium. This procedure removes the matrix from the surface of the microwell plate 650 and restricts the presence of matrix to individual microwells, as shown in FIG. 6E.

The analytes are eluted from microbeads using previously disclosed procedures. For example, photoelution, low pH and digestive compounds may be used to achieve analyte elution. Photoelution is a highly convenient method of the analyte elution in this configuration. For elution utilizing low pH, acidification of the liquid medium inside the microwells may be achieved for example via a gas phase by exposing the microwell plate to a vapor produced by concentrated (50% to 95%) trifluoroacetic acid. Alternatively, the microwell plates with loaded beads and matrix microcrystals may be dipped, soaked or otherwise exposed to a low pH liquid medium. Digestive compounds may be delivered into the microwells either before or after loading of analyte-conjugated microbeads 630.

After the analyte elution from beads 630, the liquid medium 620 is removed from the microwells by evaporation, as shown in FIG. 6F. As the evaporation occurs near the surface, a layer 660 comprising eluted analytes mixed with matrix microcrystals is formed near the surface of microwells, which is accessible to the ionization beam of the mass spectrometer. In order to improve analyte adsorption to matrix crystals, the microwell plate may be subsequently exposed to the vapor containing acetonitrile (between 40 and 95% v/v) and trifluoroacetic acid (between 1 and 10% v/v). In an embodiment, the duration of the vapor exposure is between 5 min and 10 min. In an alternative embodiment, the duration of the vapor exposure is between 15 min and 1 hour.

The disclosed method of matrix application provides at least one or more of the following advantages: 1) The experimental protocol is simpler compared to spray deposition of MALDI matrix and requires no droplet-generating equipment. 2) Localization of the matrix within microwells guarantees that the analyte signal is recorded from a single microwell and ensures no spot overlap. 3) The method has greater tolerance for impurities, such as detergents or glycerol, because it does not require matrix crystallization, which is normally inhibited by such impurities. 4) Equal amounts of ionization matrix are deposited into each microwell. 5) The method is highly scalable: microbeads of different size, from hundreds of microns in diameter to less than 1 micron may be measured by providing microcrystals of ionization matrix, or suitable nanoparticles of appropriate size. 6) A larger fraction of eluted analytes can be transferred from a bead to the surface of a microwell plate by the directional flow of liquid medium (solvent) toward the opening of a microwell during the evaporation step.

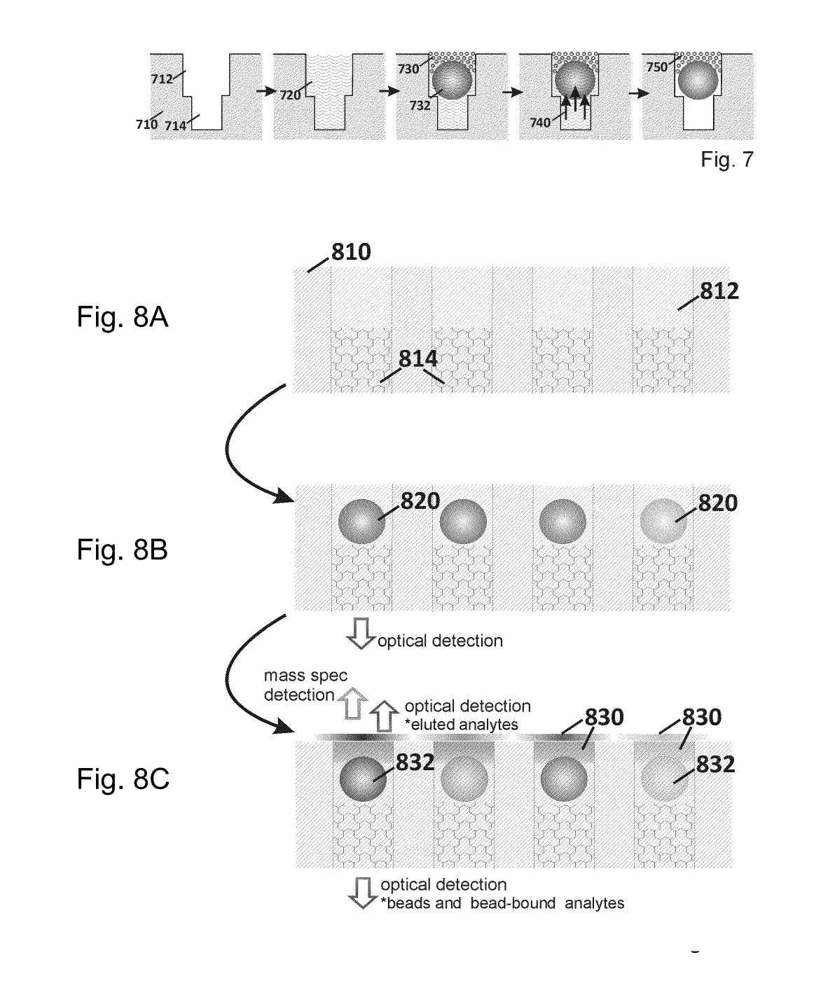

The latter principle is illustrated by way of a nonlimiting example in greater detail in FIG. 7. In this embodiment, individual microwells of microwell plate 710 comprise at least two different chambers connected to each other. The surface-proximal chamber 712 can accommodate a single microbead as disclosed previously. The lower chamber 714 is connected to the top chamber and its shape and dimensions prevent microbeads from occupying this volume, although the liquid medium can move freely between the two chambers. Methods of fabricating microwell plates with microwells featuring the disclosed design are known in the art. The shape and dimensions of the lower chamber may vary. In an embodiment, the ratio of the lower chamber volume to the top chamber volume is between 1:10 and 10:1. In an embodiment, the lower chamber is a microchannel. Both chambers are filled with the liquid medium 720 such as deionized water before microbeads are placed into the microwells. Microbeads 732 and solid phase matrix microcrystals or nanoparticles 730 are then loaded inside the chambers 712, 714 of microwells.

Lower chamber provides a reservoir for liquid medium (solvent) that carries analytes eluted from microbeads 732 toward the surface of microwell array plate upon evaporation, as indicated by arrows 740. In an embodiment, providing a lower chamber increases the fraction of analytes concentrated in the surface-proximal layer 750 accessible to the ionization beam of the mass spectrometer. In this approach individual wells represent miniature chromatographic microcolumns capable of performing elution from a single bead.

It should be noted that each of the steps described above and schematically depicted in FIGS. 3A-3C may be performed simultaneously for all members of the bead library resulting in significant time savings when a large number of beads are processed. The amount of analytes removed from beads and deposited in the microarray spots may vary depending on the specific procedure employed, as long as it is sufficient to be detected and analyzed by desired analytical methods. Furthermore the microwell array plates and the entire process may be compatible with detection by various analytical methods including optical spectroscopy and MALDI mass spectrometry.

Analysis of Fabricated Arrays of Microspots by Mass Spectrometry

Analytes eluted from microbeads and localized in spots at the surface of solid support may be measured by various methods of desorption ionization mass spectrometry. In an embodiment, the analytes are measured by MALDI MS, for example MALDI TOF MS. In an embodiment, the analytes are measured by MALDI TOF MS in the high lateral resolution mode, for example the raster distance between adjacent points probed by the mass spectrometer is between 20 and 100 microns in both x and y directions. In an embodiment, the mass spectra recorded at high lateral resolution are associated with their respective two-dimensional coordinates on the solid support, i.e. the data acquisition is performed in the MS imaging mode. Scanning in both microscope and microprobe MS imaging mode may be utilized to measure the fabricated arrays of microspots. The acquired mass spectral data may be further stored and analyzed as an image. Alternative embodiments may be contemplated that are within the scope of the present disclosure. For example, alternative forms of analyte ionization including nanoparticle—based MS, desorption—electrospray ionization MS, desorption—ionization on silicon, nanostructure-initiator MS and other techniques may be utilized to measure the arrays of analyte microspots of the present disclosure. While using the alternative techniques of analyte ionization will require specific modifications in the material, geometry and surface properties of the solid support, on which the microparticles are arrayed, as well as specific modifications of the analyte elution and immobilization protocols, such modifications will be apparent to a person skilled in the art.

Analysis of Fabricated Arrays of Microspots and Microbeads by Optical Spectroscopy

In an embodiment, the solid support and the array of microspots formed by analytes eluted from microbeads are compatible with methods of optical spectroscopy. The methods of optical spectroscopy may include absorption, transmission and reflection visible, infrared and ultraviolet spectroscopy, fluorescence and luminescence spectroscopy and numerous variations of the above techniques, e.g. immunofluorescence and chemiluminescence. In an embodiment, the solid support and the array of microspots of the present disclosure are also compatible with the methods of optical imaging.

In an embodiment, to facilitate compatibility with optical detection, the solid supports of the present disclosure are transparent in the desired wavelength range and/or have negligible autofluorescence. The methods and devices of the present disclusre in various embodiments enable: (1) optical measurements of the eluted analytes; (2) optical measurements of non-eluted analytes on microbeads and optical labels attached to microbeads; (3) making distinction between eluted and non-eluted analytes and (4) integration of acquired optical and MS data.

The types of solid support suitable for performing optical detection of libraries of microbeads are collectively known as fiber optic microwell array plates or fiber optic microwell arrays. Individual microwells that are functionally connected to one or more optic fibers represent individual analytical sites. In an embodiment, a combination of an individual microwell and a surface area surrounding the opening into the microwell represents an individual analytical site. The design, fabrication and use of fiber optic microwell array plates in various bioassays have been documented in the prior art. However, these devices have not yet been used in applications utilizing mass spectrometric detection or applications utilizing dual optical and mass spectrometric detection.

FIG. 8A, FIG. 8B and FIG. 8C show a schematic illustration of embodiment optical and MS readout channels using fiber optic microwell plates. One of the main distinctive features of fiber optic microwell array plates 810 is the ability to measure optical properties of microbeads and bead-bound analytes 820 directly inside microwells 812. As shown in FIG. 8A, a single fiber optic channel or a network of fiber optic channels 814 may be disposed in individual microwells 812 for direct contact imaging of the content of microwells 812. Using a network of fiber optic channels 814 for direct contact imaging of the analytes from beads 820 placed in microwells 812, as shwon in FIG. 8B, and other content of microwells 812 may generate high-quality high-resolution data for every microwell 812 with minimal signal interference from analytes in the adjacent microwells.

In an embodiment, experimental procedures disclosed in this specification, namely elution of analytes from beads located inside microwells, transfer of eluted analytes to the surface of fiber optic microwell array plates and localization of eluted analytes in discrete spots at the surface of the microarray plates may result in fabrication of an array of microspots containing eluted analytes 830, which is congruent and complementary to the array of beads inside the microwells 832, as shown in FIG. 8C.

As described in detail in the Experimental Examples, optical properties of the bead array and the eluted analyte array fabricated on fiber optic microwell array plates may be measured independently, for example by acquiring spectral data from the opposite surfaces of the microwell array plate (i.e. from the fiber optic bottom and the open-well top surfaces, respectively) using varying focus distance settings of the fluorescent scanner. As a result, two independent optical images may be acquired that enable analysis of the eluted analytes 830 and separately analysis of the non-eluted analytes and the microbeads themselves 832. Performing data acquisition in the imaging mode will enable direct comparison of the two sets of optical data. The experimental examples demonstrate that mixing eluted analytes with the MALDI matrix does not preclude acquisition of high-quality fluorescence signal from these analytes.

Optical data may be also acquired from beads and bead-bound analytes 820 after beads are loaded into the microwell array plate but before the elution step. The acquired data set will reflect optical properties of all analytes present on beads including analytes that may be eluted in subsequent steps. In this implementation, data acquisition from the top and bottom of the fiber optic microwell array plate is not expected to generate substantially different data sets, although the signal acquired from the bottom of the plate via the fiber optic channels may be of higher quality.

The comparison of optical images of eluted versus non-eluted analytes and comparison of optical images of analytes before versus after the elution may be used to perform quality control of the elution protocol, i.e. to measure the extent of analyte elution from microbeads including the ability to perform quantitative measurements. It also may be used to probe the structure of analyte complexes on beads, in particular when different elution reagents, e.g. digestive compounds and different elution conditions are applied to identical microbeads.

Furthermore, the comparison of acquired mass spectrometric and optical data can be used to perform more detailed study of the analyte—bead complexes than possible by the either technique alone. To facilitate such comparison, both sets of data are preferably acquired and stored as image data sets.

The compatibility of the microwell array plate with the optical spectroscopic methods may provides multiple possibilities to modify the spectral properties of beads for the purpose of distinguishing individual beads. For example, a combination of fluorescent dyes may be embedded in the bead material to provide a unique signature serving as the bead identification tag.

Integration of Flow Cell Technologies with MS Analysis

In an embodiment, the disclosed combination of a microwell plate and beads located inside individual microwells constitutes a flow cell, i.e. an array of miniature reaction vessels suitable for a variety of microfluidic applications. In this configuration, beads may be conjugated to specific reagents i.e. molecules capable of interacting with another molecule or molecular complex, which is introduced by applying a suspension or solution containing such reactant to the microwell array plate. Although beads are normally located below the surface, molecular diffusion allows the reactants to traverse that distance and reach the beads inside the microwells. Molecular reactions that occur on beads inside microwells (affinity binding, intermolecular complex formation, substrate modification by an enzyme, etc) are normally detected by optical methods using the fiber optic channel readout. Upon reaction completion, the unbound reagents are removed and the solvent replaced with another solvent. The steps of introducing and removing reactants may be repeated multiple times resulting in multiple reactions performed in the same volume over a specific time course.

The flow cell technology comprising an array of microbeads placed into microwells has been implemented in several microfluidic devices including flow cells used for massively parallel DNA sequencing. The DNA pyrosequencing performed on beads inside microwells has been documented in numerous publications and US and international patents.

Although microfluidic devices utilizing a combination of microbeads and microwell array plates are best known for the massively parallel sequencing applications, there are no fundamental restrictions that would limit their use to DNA sequencing. For example, various enzymatic reactions may be performed on bead-conjugated substrates, affinity binding may be performed using bead-conjugated affinity probes and intermolecular complex formation between subunits conjugated to beads and subunits present in the solution may be probed. The reactants in these reactions may include peptides, peptoids, proteins, protein complexes, nucleic acids, lipids, carbohydrates, small molecules, etc. These reactions may be monitored in real time or off-line by optical imaging of the bead arrays via fiber optic channels. Various methods of luminescence or fluorescence imaging may be implemented to provide qualitative and quantitative readout of these reactions.

Microwell array plates are able to retain individual agarose beads placed inside microwells even without formation of a chemical bond between the beads and the plate. In fact, once the 34 micron agarose microbeads are loaded inside the respective microwells (50 to 55 micron deep, 42 micron diameter), their removal from microwells is difficult, if not impossible. This fact suggests that beads loaded inside microwells of specific diameter will retain their positions on the microarray through repeated exposure to different solutions and washing steps, which is an essential requirement for microfluidic applications. In an embodiment, the ratio of the microwell diameter to the bead diameter that is sufficient to retain beads inside microwells is between 1.1 and 1.3 and the ratio of the microwell depth to the bead diameter is between 0.8 and 2.0. Additional modifications that further ensure fixed position of beads on the microarray include, for example placing a layer of smaller microparticles on top of every bead, using swellable beads, using compressible beads, using magnetic beads and magnetic field to retain beads, and forming a linkage between the bead and the microwell.

The technology of analyte transfer from beads onto the solid support, which is the focus of the present disclosure, can enable mass spectrometric readout of chemical reactions that occur in flow cells comprising an array of reactive microbeads and a microwell array plate. Specifically, reagents conjugated to microbeads may interact with samples introduced into such flow cells in the form of suspension or solution. A series of reactions may be performed on the same bead array by introducing different reagents into the flow cell. Individual reactions that occur inside the flow cell may be probed by optical methods using experimental techniques known in the art, for example fluorescence or chemiluminescence. In order to perform the mass spectrometric readout, the analyte elution from beads is performed in MS-compatible medium, for example deionized water. The analyte elution from microbeads and MS detection of eluted analytes may be performed using previously disclosed techniques. The MS data may be used to determine identity of probes conjugated to beads, i.e. to perform decoding of the bead array. The MS data may be also used to measure modifications of bead-conjugated reagents, for example modifications of peptide substrates by specific enzymes. The MS data may be also used to measure binding of specific molecules to bead-conjugated reagents. In an embodiment, the microwells of the disclosed flow cells may be replaced with microchannels that are nevertheless capable of retaining individual microbeads at a specific distance from the surface.

A person skilled in the art will recognize that there exist numerous other possibilities of combining bead-based chemical reactions with mass spectrometric detection of such reactions, which are made possible by the techniques of the present disclosure. An example of applications that may utilize the present disclosure is emulsion-based methods, in particular in-vitro compartmentalization. In this approach, chemical reactions are performed in individual droplets or emulsions generated by mixing aqueous and oil phases. Upon the reaction completion the individual droplets are broken, their contents are released and the generated products are analyzed by appropriate analytical methods. Known methods of droplet generation enable addition of microbeads to individual droplets. The microbeads may be conjugated to a specific reagent, e.g. a DNA that serves as a template for in-vitro transcription/translation reaction, or a peptide that serves as an enzyme substrate. The microbeads may be also conjugated to affinity reagents to capture the products of chemical reactions that occur inside the droplets. Methods of breaking droplets to release the enclosed microbeads are known, however the analytes attached to the microbeads are not typically analyzed by mass spectrometry. Accordingly, methods of the present disclosure enable high-throughput mass spectrometric analysis of microbeads from emulsion-based reactions. In particular, the present disclosure may be useful in combination with methods that are collectively known as molecular evolution or directed evolution.

Methods of Measurement and Analysis of Microarrays by Mass Spectrometry

The embodiments disclosed below relate generally to the field of high-throughput biological assays and more specifically to the field of microarrays and mass spectrometry imaging. They also relate to the field of microarray data analysis.

Mass spectrometry (MS) is a versatile analytical method, which measures interaction between charged ions and electric field of the instrument. Many MS instruments also provide a mechanism for analyte ionization. Two major techniques of analyte ionization used for the detection of biological samples are: ElectroSpray Ionization (ESI) and Matrix-Assisted Laser Desorption-Ionization (MALDI). In the ESI workflow, samples are analyzed on-line, i.e. they are prepared, introduced into the instrument and measured within a short period of time. In contrast, the MALDI workflow allows samples to be prepared and archived for the analysis at a later time, i.e. measured off-line. The MALDI method also allows the same sample to be measured more than once, if the sample contains sufficient amount of analyte. The off-line detection capability allows MALDI MS to be used in combination with other analytical methods, such as Secondary Ion Mass Spectrometry (SIMS), autoradiography, optical imaging and surface plasmon resonance. A variety of other ionization techniques of biological samples are known, including Desorption Electrospray Ionization (DESI), Desorption Ionization on Silicon (DIOS), Nanostructure Initiator MS (NIMS) and Nanostructured Laser Desorption Ionization (NALDI). Many of the above methods utilize laser desorption- ionization of samples from a solid support similarly to MALDI.

Analytes measured by conventional desorption-ionization mass spectrometry, for example MALDI TOF MS, are usually deposited in discrete spots on the flat surface of a plate made of an ionization-compatible material. Areas between spots contain no analyte. The plates may have up to several hundred individual analyte spots and multiple additional control spots. An area occupied by a single spot may be larger than the area, from which a mass spectrum is acquired. To obtain data, which is representative of an entire spot, multiple mass spectra are acquired from different positions within the spot and co-added or averaged to produce the final spectrum. The acquired data is stored in the computer memory as a mass spectrum. Each mass spectrum is usually associated with its respective location on the sample plate. The spot location serves only to provide information about the identity of samples deposited on the plate; in general no correlation is expected between mass spectra collected from adjacent spots.

Mass Spectrometry Imaging (MSI) is a method of acquiring MS data in the high lateral resolution mode. For example, MSI enables measurement of distributions of biomolecules within biological tissues, organs or even entire organisms. In this approach, mass spectra are collected within a selected area from multiple closely spaced spots, the size of individual spots being determined in part by diameter of the instrument ionization beam. The position of the ionization beam usually remains fixed during the data acquisition from each spot. The MSI data is stored and analyzed as an image file, which is a collection of individual mass spectra associated with their respective coordinates. The coordinates unambiguously link a mass spectrum with its location within the measured area. The multidimensional MSI data can be visualized as a series of images showing distribution of signal intensity for a specific mass channel or a group of mass channels. The MSI images can be correlated with data obtained by other imaging techniques, for example fluorescence imaging. Methods of MSI applied to the tissue imaging are disclosed, for example, in U.S. Pat. No. 5,808,300, U.S. Pat. No. 6,756,586 and U.S. Pat. No. 7,655,476.

Despite its success in the tissue imaging applications, desorption-ionization MSI and mass spectrometry in general have not yet emerged as a reliable readout tool for measuring biological microarrays. Several studies have reported using Secondary Ion Mass Spectrometry (SIMS) to image printed DNA and protein microarrays. However, SIMS does not allow direct measurement of analytes with molecular weight above approximately 1 kDa and therefore its current use is limited predominantly to measuring microarray morphology. A recent report has described the use of MALDI MSI to characterize a planar peptide microarray (Greying et al. Langmuir 2010). That study utilized MSI solely to perform quality control of the microarray fabrication process, e.g. to assess morphology and chemical composition of individual spots printed on the microarray. A number of studies have used MALDI MS and MALDI MSI to detect interaction between affinity probes, which are printed, i.e. immobilized on the surface of an MS-compatible microarray slide, and their respective target analytes, for example (Evans-Nguyen et al. Anal Chem 2008), also disclosed in U.S. patent application Ser. No. 12/918,399. This technique and other known methods, such as affinity SELDI TOF MS, perform biochemical reactions and mass spectrometric detection on the same solid support.

An alternative and potentially more effective method of measuring bioassays in the multiplex format involves spatially separating the microarray reaction from the downstream analysis by MS. In this approach biochemical reactions may be performed on a solid support that is optimal for biological interactions while the reaction readout is performed on a solid support that is optimal for mass spectrometric detection, for example by desorption-ionization MS. Such approach may be extended to the measurement of multiplexed reactions performed on microbeads, e.g. suspension bead arrays or planar bead arrays. Upon the reaction conclusion one or several analytes are transferred from each microbead onto an MS-compatible solid support and measured by mass spectrometry. Because the transfer of analytes onto the MS-compatible solid support is performed under controlled conditions, the acquired mass spectrometric data will be indicative of the structure of analytes on beads. In addition to probing biochemical reactions, screening of samples on microbeads by mass spectrometry may be performed for many other reasons, for example to probe quality of biologically active compounds conjugated to micro- or nanoparticles used as drug delivery vehicles. Furthermore, mass spectrometric screening of individual microbeads may be used to measure samples, which are concentrated and purified from complex biological sources using the single-bead affinity chromatography method.

There is a strong demand for the development of robust methods for high- throughput screening and analysis of bead-conjugated compounds in a microarray format using readout techniques that enable direct analyte detection, such as MALDI TOF MS. Additionally, there is a strong demand for the development of hybrid analytical technologies for measuring bead arrays that combine mass spectrometric and optical readout.

However, mass spectrometric detection of analytes directly from beads remains problematic. Although high lateral resolution imaging of compounds immobilized on individual microbeads by SIMS is known, the latter technique is limited to detecting secondary ions in the low MW range and is not suitable for the direct analysis of biological compounds, e.g. peptides, proteins and lipids. For MALDI TOF MS it is possible to use the UV laser beam of the instrument to cleave and ionize individual compounds if they are conjugated to microbeads via photosensitive linkers, however this approach has limited value because analytes may be conjugated to the microbeads by linkers of a different nature, for example acid-labile bonds, which are not cleavable by UV irradiation.

Methods are known in which analytes are released from individual microbeads, placed on MS-compatible surface, such as MALDI target plate, and measured by MALDI TOF MS. These methods are largely manual and therefore limited to the analysis of a single microbead or several microbeads at a time.

On the other hand, methods of the present disclosure enable simultaneous transfer of multiple analytes from bead arrays comprising thousands to millions of microbeads onto the surface of a solid support. In particular, these methods enable fabrication of an array of analyte-containing microspots on a solid support that is complementary and congruent to the precursor array of microbeads. There exists a direct spatial relationship between individual beads within the bead array and individual microspots containing analytes released from the respective beads. This relationship enables the use of mass spectrometric data acquired from the microspots of eluted analytes to determine the identity of samples originally present on the microbeads.

The processes and methods of the present disclosure enable the use of mass spectrometry, in particular MALDI TOF MSI, to directly measure analytes deposited on a solid support as an array of microspots including arrays fabricated from libraries of microbeads. The disclosed methods also enable the use of mass spectrometry to obtain detailed information about the array morphology, including detection, identification and assignment of individual spots, mapping of the spot locations within the microarray and determination of the size, shape and degree of overlap for individual microarray spots. The disclosed methods also enable the use of mass spectrometry, in particular MALDI TOF MS, to obtain information about the presence and co-localization of analytes within specific spots on the microarray and the relative amounts of analytes in those spots. The disclosed methods also enable detailed analysis of analytes on the microarray by mass spectrometry using, among others, post-source decay (PSD) and collision-induced dissociation (CID) fragmentation mechanisms. The disclosed methods also enable the use of mass spectrometry to perform two or more consecutive measurements of the same microarray using different acquisition parameters or even different instruments for the purpose of detailed characterization of the analytes. The disclosed methods also enable direct comparison of the microarray images obtained by mass spectrometry and by other analytical imaging methods, for example optical imaging, and the use of optical imaging data to guide the mass spectrometric data acquisition and analysis. The disclosed methods also enable the use of mass spectrometry microarray data for the detection of interaction between various biomolecules. The disclosed methods also enable the use of quantitative mass spectrometry in the microarray format. The disclosed methods also enable the use of mass spectrometry to perform detection of analyte modifications in the microarray format. The presently disclosed embodiments also provide a data structure that facilitates analysis of the microarray MS datasets. Methods of the present disclosure facilitate analysis of various biological arrays using the technique of mass spectrometry imaging.

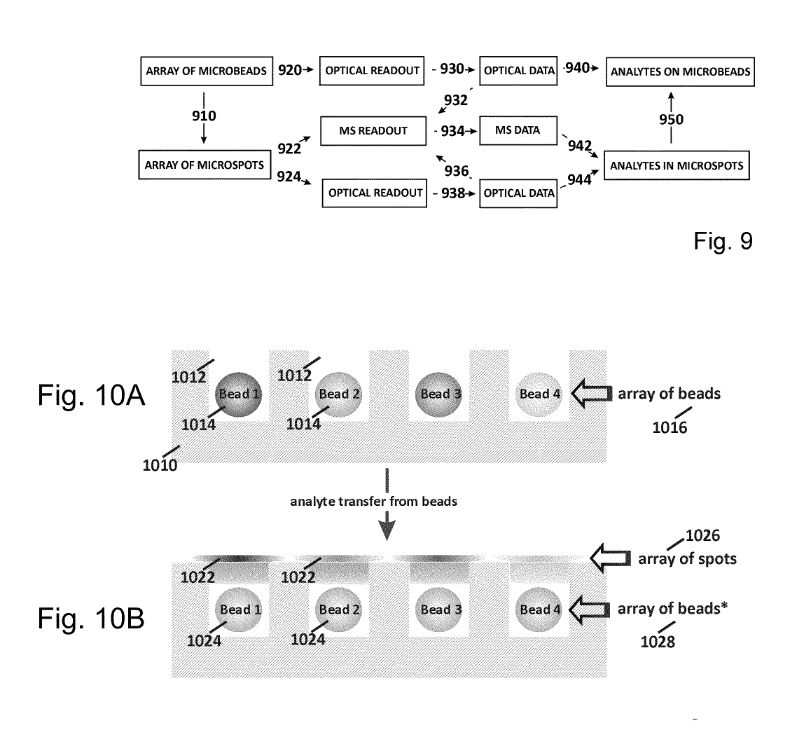

The flow diagram in FIG. 9 depicts relationships between individual elements of a mass spectrometric assay according to embodiment methods of the present disclosure. The arrow 910 denotes a process of fabricating an array of microspots from an array of microbeads. The arrow 920 denotes a process of optical readout from the array of microbeads. The optical readout may be performed both before and after the fabrication of the array of microspots. The arrow 922 denotes a process of mass spectrometric readout from the array of microspots. The arrow 924 denotes a process of optical readout from the array of microspots. The arrow 930 denotes a process of producing an optical data set from the array of microbeads. The arrow 932 denotes a process of using the optical data acquired from the microbead array to guide the mass spectrometric data acquisition. The arrow 934 denotes the process of producing a mass spectrometric dataset from the array of microspots. The arrow 938 denotes a process of producing an optical data set from the array of microspots. The arrow 936 denotes a process of using the optical data acquired from the microspot array to guide the mass spectrometric data acquisition. The arrows 942 and 944 denote the processes of analyzing mass spectrometric and optical data, respectively, to identify analytes present in individual microspots. The arrows 940 and 950 denote the processes of analyzing data from the array of microspots and from the array of microbeads, respectively, to identify analytes originally present on individual microbeads.

Fewer elements than shown in FIG. 9 may be present in some assays performed using methods of the present disclosure. In fact, elements related to the optical readout of analytes are not required in order to utilize many procedures of the present disclosure.

Analytes to be measured by mass spectrometry are provided in the form of an array on a solid support. In an embodiment the solid support is a microwell plate. The plates may be manufactured from various materials including unmodified and modified silicon, glass, chemically modified glass, plastics, polymers, resins, metals and the composite materials. In an embodiment, the surface of the solid support contains a thin layer of material that is non-reactive, optically transparent and electrically conductive, for example a 5 nm layer of Gold. The microwells may be arranged in a specific order, for example a hexagonal or square grid. The dimensions of microwells may vary. In an embodiment, the microwells are 42 μm in diameter and 55 μm deep with the 50 μm distance between the centers of adjacent microwells. In an embodiment the plates are glass fiber optic microwell plates that enable optical readout from analytes inside microwells via fiber optic channels. The dimensions of microwell plates may vary. In an embodiment, the plates have dimensions of a standard microscope slide, approximately 75×25×1 mm. In another embodiment, the plates have dimensions of a 384-well plate, approximately 128×86×1 mm. Microwell plates of the disclosed dimensions fit into standard plate loading devices of the commercial MALDI mass spectrometers and may be further secured using slide adapters, such as microscope slide adapters utilized in the tissue imaging applications that are available commercially from various vendors, for example HTX Imaging (Carrboro NC).

The disclosed methods are compatible with all analytes that are detectable by desorption-ionization mass spectrometry. The examples of analytes are a polypeptide, a protein, a peptidomimetic, a nucleic acid, a lipid, a carbohydrate, a small molecule, and their combinations. The analytes may be extracted from natural sources, produced by in-vivo or in-vitro synthesis methods, or produced or modified by chemical or biochemical methods. The analytes may be molecular complexes comprising two or more distinct molecules or may be fragments of precursor molecules produced for example by enzymatic digestion. The analytes may have additional properties, which are measured by techniques other than mass spectrometry, for example have distinctive optical spectra. The presently disclosed embodiments are compatible with various ionization matrices known in the mass spectrometry field. For example, known MALDI ionization matrices such as α-cyano-4-hydroxycinnamic acid (CHCA), sinapinic acid (SA), 2,5-dihydroxybenzoic acid (DHB), or their combinations may be used. Furthermore, liquid ionization matrices suitable for UV or IR MALDI or alternatively nanoparticles that promote ionization of specific analytes may be used.

In an embodiment, desorption-ionization mass spectrometry is used to measure an array of analyte-containing microspots, which is fabricated by transfer of analytes from a planar bead array, i.e. a library of microbeads or similar microparticles randomly arrayed on a microwell plate. In an embodiment, no more than one bead occupies a single microwell. FIG. 10A schematically depicts a cross-section of a small area of an embodiment microwell array plate 1010 with individual microwells 1012 occupied by microbeads 1014, which form an array 1016. In reference to FIG. 10B, transfer of analytes from beads results in fabrication of microspots 1022 that form an array 1026. Individual microbeads 1024 are retained on the microwell plate after the analyte transfer and form a spatially related array 1028. The array of microspots 1026 is located on or near the surface of the microwell plate and is detectable by mass spectrometry. Procedures utilized in the transfer of analytes from the bead array to the microspot array enable selective release of specific analytes from the microbeads. The extent of the analyte transfer from individual microbeads may vary. Furthermore, the distance between individual spots may vary and there may be some degree of overlap between adjacent spots. Preferably the area of a microarray occupied by two or more overlapping spots represents no more than 25% of the total microarray area.

In an embodiment, analytes released from microbeads are localized near their respective beads, however the extent of their localization on the microwell plate may differ. The released analytes may remain very close to their respective beads, for example be confined to the outer layer of a bead. In an embodiment, the released analytes may be localized within a single microwell. In an embodiment, the released analytes may “spill over” from the microwells and be present on the surface of the microwell plate between openings into the wells. Methods of analyte release from beads, which are disclosed above, may enable precise control over the extent of the analyte migration on the microwell plate.

In an embodiment, analytes localized in the array of microspots may be mixed with ionization matrix. The ionization matrix may be commonly used MALDI matrix, such as CHCA, SA or DHB. The ionization matrix may be also a liquid matrix including ionic liquid matrices. In an embodiment, various nanoparticles, nanostructures and nanomaterials known in the fields of nanoparticle-assisted mass spectrometry and surface-assisted mass spectrometry including nanoparticles developed for a specific group of analytes may be used as an ionization matrix. The matrix may be deposited throughout the array area or localized in specific spots. Furthermore, other known methods of desorption-ionization including matrix-free methods may be utilized in conjunction with the methods of the current disclosure.

Methods of the present disclosure may also enable mass spectrometric readout directly from beads inside the microwells, i.e. without the prior transfer of analytes. The analyte release from a bead may be achieved by photolysis of the photosensitive analyte-bead linkages induced by the laser beam of the mass spectrometer striking the bead. In this approach, the array of analyte spots will coincide with the array of microbeads.

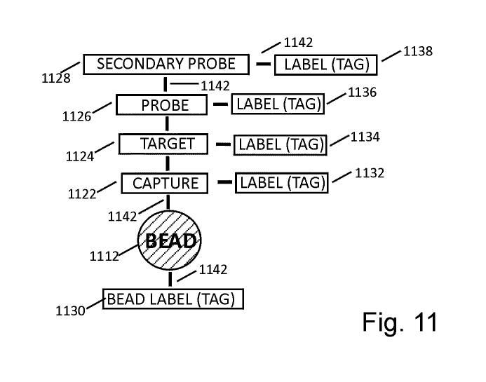

There exist a large number of different bead designs and bead assays that may be measured in the microarray format by mass spectrometry according to the presently disclosed embodiments. FIG. 11 is a schematic representation of analytes that may be present on individual microbeads, for example beads used in affinity binding assays. In this depiction the bead 1112 is conjugated to a capture molecule, or a molecular complex 1122. The capture molecule is conjugated to a target molecule, or a molecular complex 1124 that may be conjugated to a probe molecule, or a molecular complex 1126 that may be further conjugated to a secondary probe molecule, or a molecular complex 1128. The bead may be additionally conjugated to a bead label or bead tag 1130. Additionally, tags may be conjugated to the capture molecule as the capture label 1132, target molecule as the target label 1134, probe molecule as the probe label 1136 and secondary probe as the secondary probe label 1138. Each of the labels or tags may be a specific molecule, a molecular complex, or a group of several distinct molecules serving as a mass barcode. The conjugation between individual elements is achieved by means of linkages 1142, which may be a specific chemical bond, a molecule or a molecular complex. The linkages between different elements may be of the same or different nature. The linkages may be stable or labile, for example, photo-labile, acid-labile or heat-labile. The linkages may contain protease-sensitive chemical bonds. Additional elements may be also present on beads. Some of the elements may have additional properties, such as fluorescent or luminescent properties. Some of the elements may be designed specifically for the detection by mass spectrometry or by other techniques, for example fluorescence, luminescence, Surface Plasmon Resonance or autoradiography. More than one type of target molecules may be conjugated to a capture molecule. Individual elements on beads may contain additional embedded labels, which are inseparable part of their chemical structure. The examples of embedded labels are stable-isotope labeled atoms or chemical groups covalently attached to the target molecules, such as ICAT reagents.

The fabrication of arrays of microspots from bead libraries is described in detail above. In general, this process involves disruption of linkages between individual components within the analyte-bead construct and localization of analytes in distinct spots on a solid support. In an embodiment, the transfer of analytes is performed under conditions that ensure co-localization of analytes released from the same bead. The transfer of analytes is also preferably performed under conditions that preserve relative concentrations of analytes, thereby allowing quantitative measurements of bead libraries by mass spectrometry. The disruption of linkages may be achieved by various means, for example, exposure to an acidic medium, exposure to light, exposure to digestive enzymes, exposure to heat. Once the analytes are released from beads, they may be measured by mass spectrometry. There exist a virtually unlimited number of different protocols for the analyte transfer from beads onto the microarray depending on a particular bead design and the experimental setup. depending on the nature of reagents used to disrupt the linkages, the concentration of reagents, duration of exposure and the order, in which the reagents are applied, among other parameters. Accordingly, the number and chemical composition of elements, which are transferred from a bead onto a microarray, may vary. For example, some or all of the analytes shown in FIG. 11 may be transferred from beads onto an array of microspots. Some elements may remain conjugated after the transfer from beads onto the microarray, if their respective linkages are not disrupted. On the other hand, some elements may undergo additional internal fragmentation during the transfer onto the microarray, for example due to the exposure to a digestive enzyme. When a particular analyte undergoes fragmentation, all or some fragments of an original analyte molecule may be present on the microarray. Furthermore, some of the analytes may undergo additional fragmentation during the mass spectrometric measurement, for example via mechanisms known as post-source decay (PSD), collision-induced dissociation (CID) and neutral molecule loss. However, despite the presence of multiple analytes in each microarray spot, the high mass resolving power of mass spectrometry allows these analytes to be measured simultaneously and distinguished based on their molecular weight.

Each type of beads within the bead library may exist in multiple replicates, so that spots containing identical analytes are present in multiple locations throughout the microarray. The number of replicates for each bead type is preferably between 2 and 10,000 and more preferably between 10 and 1,000.

Additionally, various control spots may be present within a microarray. The control spots may contain analytes of known molecular weight and be used for the calibration of mass spectrometer. The control spots may also contain known amounts of analytes and be used for example to determine optimal intensity of the ionization beam, determine optimal sensitivity of the instrument detector and optimize the instrument performance. The analytes in control spots may be deposited in known locations within the microarray either manually or by spotting instruments. Some of the control analytes may be also deposited throughout the entire microarray area, for example as a mixture with MALDI matrix. In addition to control spots, which are deposited directly on a microarray, beads conjugated to control analytes may be included in the bead library, from which a microarray is produced. Such “control beads” may additionally contain analytes, which are measured to provide quality control of the process involved in the fabrication of a microarray from the bead library. For example, the “control beads” may be used to assess various conditions of the analyte elution from beads and fabrication of individual microarray spots, including digestion with enzymes, measure degree of co- localization of analytes within individual spots, measure quantitative ratio of analytes within individual spots, and degree of overlap of individual spots. Such beads may carry additional labels, which identify them as “control beads.”

Several distinct libraries of beads may be accommodated on a single microarray chip, i.e. solid support, so that areas, which contain spots produced from different libraries, are spatially separated. This is accomplished by depositing individual bead libraries in specific areas within the microarray chip. In this approach, the location of a particular bead library on the microarray chip is known, while the distribution of beads within each area is random. Therefore, the resulting microarrays have both positional encoding and random distribution of analytes. The microarray chips may also have features that facilitate identification of areas, which contain analyte spots. For example, there may be provided visual markings that specify analyte-containing areas of interest. The markings may also be provided in the electronic format in the form of coordinates, which specify the area of interest.

Microarray Data Available Prior to the Mass Spectrometric Analysis

A combination of two arrays is disclosed here that comprises an array of beads submerged into wells of a microwell plate and a complementary array of microspots containing analytes released from the beads. A substantial amount of information can be gathered from such microarray system that can be used to guide acquisition of the mass spectrometric data from the array of microspots.

Specifically, there exists substantial amount of information related to the composition of the bead library that was used to fabricate the bead array and the microspot array. The information may include description of specific compounds present on beads and compounds transferred from beads and localized within the microspot array. Such information may include the type of compounds, e.g. peptides, proteins, lipids, carbohydrates, etc and their molecular weight or the range of molecular weights. The information may also include the total number of distinct analytes per each bead and their role in the corresponding bead assay, e.g. bead mass tag, target, capture, probe, secondary probe, etc. Fragmentation profiles of individual analytes during the MS measurement may be provided that can be used to select optimal m/z detection range, for example to account for the post-source decay fragmentation. Also, the amount of analytes on individual beads may be approximately known, which can be used to select optimal MS data acquisition protocol, for example, adjust intensity of the ionization laser beam and the detector sensitivity. All of the above data may be provided both for the compounds that are known to be present on beads, e.g. bead mass tags and also for the compounds that are expected or may be present on beads, e.g. possible target analytes that have affinity for the corresponding bead-conjugated capture reagents.

Additional information that may be available prior to the MS data acquisition includes the total number of beads deposited on the microwell plate and the number of replicates, i.e. identical beads for each bead type.

Additional information that may be available prior to the MS data acquisition includes the sample processing history and description of the functional assay used in the fabrication of the precursor bead library. For example, phosphorylation of a peptide substrate by a kinase results in appearance of a peptide peak in the mass spectrum that is shifted by 80 Da from the precursor peak and corresponds to the molecular weight of a phosphate group. Such knowledge may be used in the selection of an appropriate m/z detection range for the mass spectrometric assay.

Additional information that may be available prior to the MS data acquisition includes description of methods used for the release of analytes from beads and their transfer to the microspots on the microarray chip. One example is the use of enzymatic digestion that reduces bead-conjugated proteins to a series of shorter polypeptide fragments that are measured in the MS reflector mode. The type of MALDI matrix and the method of its application to the solid support may be also provided.

Additional information that may be available prior to the MS data acquisition includes geometry of the fabricated bead array. The total area occupied by the bead library, its coordinates on the microwell plate and the average density of beads on the plate may be provided. Because the beads may be confined to an area smaller than the total area of the microwell plate, the boundaries of such area on the microwell plate may be visually marked or alternatively provided electronically in the form of (X,Y) coordinates. Note that the majority of MALDI MS instruments are already equipped with a high-resolution video camera that may be used to determine coordinates of the area of interest using the provided visual markings. In an embodiment, a simplified process of selecting a microarray area containing the bead library is provided that comprises: (i) depositing beads in a predefined area of the microwell plate, for example by using a gasket during the bead loading; (ii) placing and securing the microwell plate in a predefined area of the MALDI instrument target plate loading chamber, for example by using a microscope slide adapter available from commercial vendors (HTX Technologies LLC, Carrboro NC) and (iii) storing the coordinates of the area of interest in the instrument memory.

Additional information that may be available prior to the MS data acquisition includes the spatial arrangement of microwells within the microwell plate. The type of grid (e.g., hexagonal, square, rectangular etc), diameter of individual wells and the distance between centers of adjacent wells may be provided. In an embodiment, the diameter of individual wells is assumed to be approximately equal to the diameter of individual analyte spots.

If the beads or bead-conjugated compounds have distinctive optical properties, a potentially large amount of information may be obtained from optical imaging of the disclosed combination of a bead array and a microspot array that can be used to guide acquisition of the mass spectrometric data from the latter. The examples of optical imaging are fluorescence imaging and luminescence imaging. Optical imaging may be performed in three different configurations: (i) imaging of the bead array before the release of analytes; (ii) imaging of the bead array after the analyte release and (iii) imaging of the array of microspots containing analytes released from the bead array.

In an embodiment, some of the compounds with distinctive optical properties may be removed, for example washed off the bead array after the optical image has been acquired but before the analytes are eluted to form an array of microspots. The purpose of this step is to simplify and improve quality of the subsequently measured mass spectra by removing compounds that introduce additional peaks in the mass spectra possibly complicating interpretation of the MS data. Furthermore, some of the optical spectra may be acquired under conditions that are not compatible with the downstream desorption-ionization, for example optical spectra may be acquired in the presence of buffers and reagents used in chemiluminescent reactions. Such reagents may be subsequently replaced with deionized water or other suitable medium. Note that the disclosed system is sufficiently flexible to allow selective removal of specific compounds while retaining other analytes. An example of orthogonal elution procedure is removal of a fluorescently labeled antibody from beads by washing the bead array with low pH medium, which is followed by UV photorelease of peptides covalently attached to the same bead via a photolabile linker.

Two non-limiting examples of using optical data to guide the subsequent MS data acquisition are provided here. It should of course be understood that other variations are possible. In an embodiment the microbeads are optically encoded. Numerous methods of optically encoding microbeads are known in the art that are compatible with the methods of the present disclosure, for example methods that involve introducing a combination of fluorescent dyes into the bead core that is subsequently read by a fluorescence scanner. Up to 100 unique optical codes are available using a combination of two fluorescent dyes on the Luminex® platform and much greater number is possible when a combination of several dyes or quantum dots is employed. The optical bead codes serve to provide information about the nature of reagent conjugated to individual beads (optical encoding). In an alternative embodiment compounds conjugated to beads, for example the capture-target complex, are probed with a fluorescently labeled reagent, such as the target-specific fluorescent antibody. In this approach the binding of target to a capture reagent is detected in a fluorescence image of the fabricated bead array while identity of the capture reagent is determined by mass spectrometry imaging after the release of capture reagent from the beads. In a modification of the latter method, target molecules themselves may be labeled with a fluorescent reagent before binding to the bead-conjugated capture regents thereby eliminating the need for a fluorescent antibody. The optical data may be used to guide the MS data acquisition in several ways: (1) It may serve as a quality control measure to provide a rapid assessment of the extent of biochemical reactions occurring on beads and of the quality of the array fabrication procedure including the extent of analytes elution from beads and their localization on the solid support. Microarrays that fail such QC test will not be measured by mass spectrometry, which saves valuable instrument time. This approach may further benefit from the ability to include a subset of “control beads” with defined properties as disclosed above. (2) The optical data may restrict MS data acquisition to particular areas or individual spots on the array that either exhibit or lack a specific optical signal. The rationale behind this approach is that limited data acquisition saves instrument time and data storage space. (3) Importantly, the available optical data may be used to provide mass spectrometer with pixel-specific rather than region-specific data acquisition parameters for defined subsets of individual analyte spots within the array. For example, depending on the specifics of the measured optical signal and its strength, which are indicative of properties and concentrations of analytes in a particular spot, appropriate molecular weight range, number of averaged shots per spectrum, intensity of the ionization laser beam, precursor ion for MS-MS sequencing as well as other parameters may be provided for each spot. This approach may help to minimize the sample consumption and increases the likelihood of obtaining meaningful mass spectrometric data.

The disclosed methods of using optical data to guide the mass spectrometric data acquisition can be easily integrated into the MS instrument control software of existing or newly developed instruments. Known methods of image overlay including methods developed for the MS tissue imaging studies may be used to map locations of individual spots with specific optical properties. Furthermore, the currently available MS instruments are capable of positioning the sample stage at given locations with approximately 1 micron accuracy, which is sufficient for most of the disclosed analytical applications.

In an embodiment the array of microspots has been previously measured by mass spectrometry, for example SIMS or MALDI TOF MS. The Example 20 demonstrates that the amount of matrix-embedded analytes deposited on the microarray is sufficient for performing at least two consecutive rounds of MS measurements. Accordingly, substantial amount of information from the first MS data set may be available that can be used to guide the subsequent MS data acquisition. For example, the available data may include the presence of analytes of specific molecular weight, the total number of distinct analytes, morphology of individual microarray spots, etc. In an embodiment the available MS data concerning the molecular weight of compounds present on the microarray is used for the selection of precursor (parent) ion for MS-MS sequencing at specific locations. The previously acquired MS data set is preferably supplied as an image dataset, although its lateral resolution may be different from the lateral resolution of the subsequent MS scan. In fact it may be advantageous to perform an initial rapid “surveillance” scan at lower lateral resolution to quickly identify the presence of compounds of interest within a specific area, which is followed by more detailed analysis.

Additional information may be also available that is commonly utilized in the MALDI MS tissue imaging applications. This may include the type of used ionization matrix, the method of matrix application and the presence of molecular weight calibrants throughput the array or in specific positions.

Data Acquisition Parameters for MS Scan

Non-limiting methods of the present disclosure and experimental results are illustrated using MS instruments and software packages that are readily available and commonly used in the field. The spectral acquisition is performed using the microprobe imaging mode on the Applied Biosystems® 4800 MALDI TOF-TOF analyzer equipped with 4000 Series Explorer™ software. The array scanning is performed using 4000 Series Imaging software available in the public domain. It should of course be understood that that other instruments and software programs may be successfully used with the methods of the present disclosure.

In an embodiment, the methods of the present disclosure focus on optimization of the MS imaging technique to analyze arrays of analytes, specifically random arrays and more specifically random arrays fabricated from libraries of microbeads.

Prior to performing mass spectrometric measurement of an array a number of parameters may be defined and submitted to the data acquisition software. For the purpose of illustration these parameters are divided into two groups termed scan parameters and spectral parameters. The group of scan parameters determines coverage of the microarray area, from which the mass spectrometric data is acquired. The group of spectral parameters controls the instrument settings for acquiring a mass spectrum from a single location. In the examples of the present disclosure many of the scan parameters are provided within the dialog window of the 4000 Series Imaging software, while many of the spectral parameters are provided within the dialog window of the 4000 Series Explorer™.

The group of scan parameters may include the total area to be measured, position of the area within the microarray as defined by a set of two-dimensional coordinates, coordinates of a first spot and the sequence in which the spectra are acquired within the measured area. For mass spectrometers featuring variable diameter of the laser ionization beam, the beam diameter may be provided. The raster distance that determines the distance between adjacent pixels, i.e. locations probed by ionization beam of the mass spectrometer in X and Y directions may be also provided. Providing the raster distance that is smaller than diameter of the laser ionization beam enables measurement in the oversampling mode that may increase the resolution of MS imaging (Jurchen et al. Journal of the American Society for Mass Spectrometry 2005). Alternatively, the raster distance may be provided as the number of points within the measured area from which the mass spectrometric data will be acquired.

The group of spectral parameters provided for a MALDI TOF instrument may include the measurement mode (linear, reflector or MS-MS), ion mode (negative or positive), m/z detection range, spectral resolution, intensity of the ionization laser beam, number of single shot spectra averaged per spectrum, the acquisition mode (stationary or moving), precursor ion and m/z window for MS-MS detection and other parameters.

In an embodiment, in the methods of the present disclosure, the optical data acquired from an array of microbeads or from a spatially related array of analytes eluted from the microbeads is used to guide the acquisition of mass spectrometric data.

In an embodiment, rather than providing identical mass spectral acquisition parameters for all spots within a measured region, specific mass spectral acquisition parameters are provided for groups of adjacent or non-adjacent individual spots within the measured region. The specific mass spectral acquisition parameters are determined using optical or mass spectrometric data previously acquired from the same region and also using information about the composition of the bead library, sample processing history and array fabrication protocols. This approach enables effective mass spectrometric measurements of analyte arrays comprising highly diverse compounds.

In an embodiment, the spatial relationship between an array of microspots and an array of microbeads, which are fabricated on a microwell array plate, is utilized to assign analytes detected in the microspot array to individual beads within the bead array.

Acquisition of Mass Spectrometric Data from the Microarrays

The microwell array plates may be loaded into the imaging-capable MS instruments using plate adapters and sample holders, which are utilized in MS tissue imaging studies. The microarray slides may be placed on a flat surface in order to provide accurate time-of-flight mass readings. The examples of commercially available instruments capable of performing microarray imaging are Applied Biosystems ABI 4800 and 5800 MALDI TOF-TOF, Bruker AutoFlex III and Ultraflextreme and Shimadzu Axima MALDI MS. Mass spectrometers capable of imaging in the microscope mode, as described in (Klerk, Altelaar et al. International Journal of Mass Spectrometry 2009), may also be used for the microarray imaging experiments. Although the examples in the presently disclosed embodiments use mainly MALDI method of ionization and time-of-flight (TOF) method of detection, many other configurations are possible with respect to the ionization methods and analyte detection methods. For example, DESI, DIOS, LAESI, NALDI and other known ionization matrix-based and ionization matrix-free methods of desorption ionization may be used for the microarray imaging. Orbitrap, ion trap, quadrupole, FT-MS, hybrid and tandem MS may be used for the analyte detection. Commercially available MS imaging software, for example 4800 Series Imaging, or Bruker flexImagingTM may be used to select all parameters for the microarray scan and perform the scan. Prior to the microarray image data acquisition, the mass spectrometer may be calibrated and various data acquisition parameters selected.

Arrays of analytes measured by mass spectrometry may have different morphology including the size and shape of individual spots and the separation between spots. In an embodiment, the spots are approximately circular in shape, have similar size, i.e. the spot area does not differ by more than 10%, and do not overlap. In an embodiment the diameter of individual analyte spots is approximately equal to the diameter of individual microwells. The distribution of analytes within each spot may be uniform, or have a specific pattern, for example a concentration gradient. The properties of ionization beam used to measure the microarray may also vary depending upon a specific instrument. In an embodiment, the ionization beam is a laser beam approximately circular is shape. The beam diameter may be variable, for example vary between 10 and 1000 μm. The beam diameter and distribution of intensity within the beam may be controlled by the instrument software and optics.

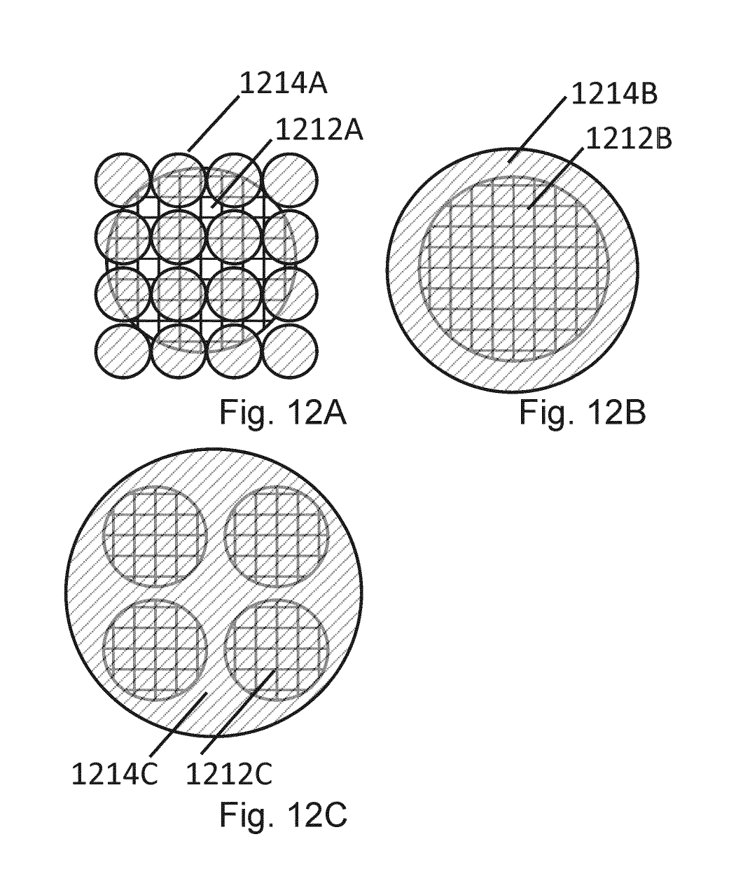

FIG. 12A, FIG. 12B, and FIG. 2C show several non-limiting readout options with respect to the size of microarray spots and diameter of the ionization beam. In an embodiment, shown in FIG. 12A, the diameter of ionization beam 1214A is smaller than the diameter of individual microarray spots 1212A, so that MS data from each spot is stored in several pixels. In this embodiment, the number of pixels per spot is preferably between 2 and 100, more preferably between 4 and 16. In an embodiment shown in FIG. 12B, the diameter of ionization beam 1214B is similar to the size of a microarray spot 1212B, but preferably is between 1.1 and 1.5 times larger than the diameter of a spot. In an embodiment shown in FIG. 12C, the diameter of ionization beam 1214C is significantly larger than the diameter of a microarray spot 1212C, such as, for example, between 1.5 and 10 times the diameter of the spots. The later embodiment represents a multiplexed readout mode of a microarray and is particularly suitable for conducting a rapid initial “surveillance” scan to quickly identify the presence of a particular analyte within a specific area of a microarray.

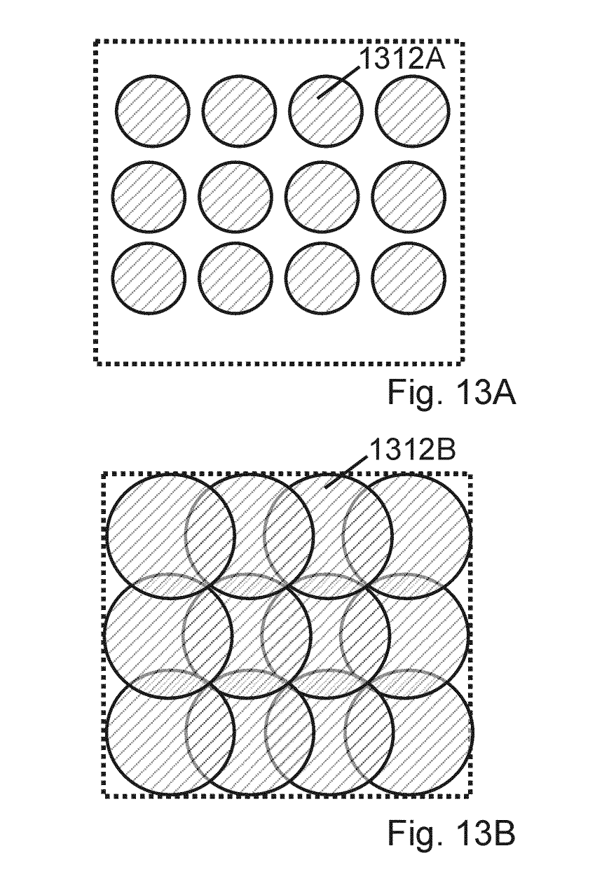

FIG. 13A and FIG. 13B show non-limiting readout options with respect to the displacement of ionization beam during the MSI data acquisition. In an embodiment, shown in FIG. 13A, the displacement of ionization beam between two adjacent positions, from which the data is acquired, is larger than the beam diameter 1312A. Consequently, the microarray data is collected from non-overlapping areas of the microarray. In this embodiment, the linear beam displacement is preferably between 1.1 and 2.0 times the beam diameter. In an embodiment, shown in FIG. 13B the displacement of ionization beam is smaller than the beam diameter 12B. Consequently, certain microarray areas are measured in two or more distinct positions of the ionization beam and information from the same sample analyte will be present in two or more pixels on the microarray image, which is known as oversampling. In this embodiment, the beam displacement is preferably between 0.05 and 0.95 times the beam diameter, more preferably between 0.3 and 0.5 times the beam diameter. The oversampling measurement may be performed under conditions, which result in a complete depletion of analytes in the measured spot. Therefore, a subsequent displacement of ionization beam will effectively measure an area smaller that the area covered by ionization beam.

The position of ionization beam relative to the microarray during the single spot data acquisition may remain stationary. Alternatively, the data may be collected while the ionization beam moves continuously. In the latter case, the data acquisition rate should be sufficiently fast, so that multiple spectra may be collected within an individual spot area. The data acquisition rate is controlled, among other factors, by frequency of the instrument ionization laser and the instrument electronics.

In order to perform microarray imaging, an MS instrument is provided with coordinates of individual pixels, from which the MS data will be recorded. In an embodiment, the coordinates may be supplied in two formats: (1) the scan description, or (2) a list of coordinates for individual pixels, which will be measured. Providing scan description, which is a common approach in the MS tissue imaging studies, includes providing several parameters such as scan pattern, scan type, linescan direction and linescan sequence (http://www.maldi-msi.org/download/imzml/CVimagingMSList.pdf), which together unambiguously describe the imaging experiment. In the second approach, the list of coordinates may be entered manually, or obtained from an independent source, for example a visible or fluorescent image of the microarray. The order, in which the pixels are measured, may be also provided. This is particularly important for the measurements utilizing the oversampling technique.

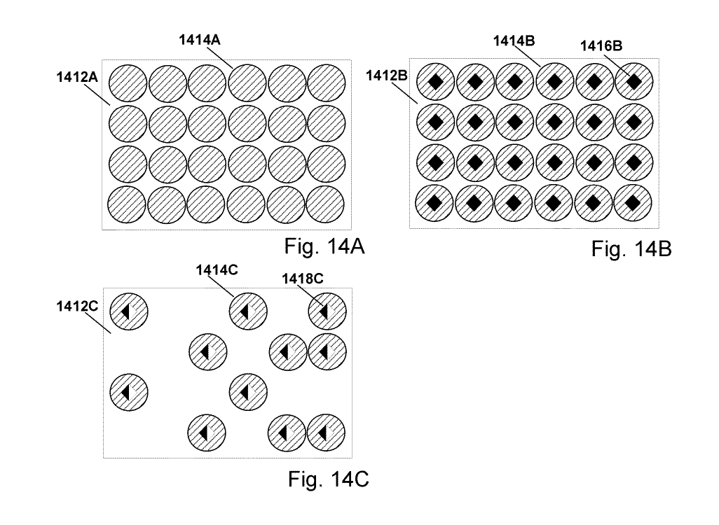

In an embodiment, the microarray imaging is performed to ensure sufficient coverage of a selected area of a microarray. By way of a non-limiting example, FIG. 14A shows a schematic representation of such scan where data is acquired from closely spaced locations 1414A within specific area 1412A. This method of data acquisition may be used when the location of individual analyte spots is not known prior to imaging. An alternative method of the microarray imaging is shown in FIG. 14B. In this example the locations of individual analyte spots 1414B are known prior to the MSI scan. The locations of individual spots may be determined from the visible image of an array, which contains specific features 1416B, for example locations of microwells. Also, known parameters of the grid of microwells within the measured area 1412B may be used to determine the scan parameters. A second alternative method of the microarray imaging is shown in FIG. 14C. In this method, the MS data acquisition is restricted to spots 1414C within the microarray area 1412C, which possess specific features 1418C, such as fluorescent signals. A smaller number of spots may be measured using this approach, which results in a reduced scan time.

While the microarray MSI data acquisition methods disclosed here use an example of a microprobe mode imaging, which is employed in the majority of current commercially available instruments, alternative methods of microarray image acquisition, such as the microscope mode described, for example, in (Klerk, Altelaar et al. International Journal of Mass Spectrometry 2009) are fully compatible with the presently disclosed embodiments.

In an embodiment, if enough analyte is present on the microarray, the same microarray area may be scanned by MSI more than once using identical or different data acquisition settings. For example, consecutive MSI measurements may be performed using linear and reflector MS mode, MS TOF and MS TOF-TOF tandem mode, different mass range, different spectral resolution, or different scan parameters. Furthermore, the microarray may be subsequently scanned using a different MSI-capable instrument including instruments that employ different ionization mechanism, e.g. SIMS.

Analysis of the Mass Spectrometric Data Acquired from Microarrays

The microarray imaging by mass spectrometry generates a complex dataset. Several methods of storing MSI data are known, for example the data may be stored in the Analyze™ 7.5 format developed by Mayo Clinic (Rochester, Minn.). In an embodiment, the stored dataset comprises at least an array of two-dimensional coordinates and an array of mass spectra, with each mass spectrum unambiguously associated with a specific location on the microarray determined by its two-dimensional coordinates. If the microarray is measured by mass spectrometry more than once, the dataset may contain the corresponding number of additional mass spectra, each mass spectrum unambiguously associated with its specific location on the microarray. If the microarray is measured by other instrumental methods, for example fluorescence imaging, the dataset may also contain fluorescence or other data.

In an embodiment, the generated microarray image represents raw data. Accordingly, numerous methods of data processing known in the mass spectrometry field and in the microarray analysis field may be applied to the generated dataset. The MS-based methods of data processing may include baseline correction, spectral smoothing, peak narrowing, removal of isotope-induced peaks, and molecular weight calibration. The MS-based methods of data processing may further include correction for the presence of contaminants, correction for the presence of multiply charged ions, and correction for the presence of salt adducts. The MS-based methods of data processing may further include correction for the different path length in the time-of-flight instruments including methods known as peak binning The above methods may be applied to the entire microarray image or selected regions and may be applied either automatically or manually. The above methods may be applied either concurrently or subsequently to the microarray MSI data acquisition.

Various methods of data processing, which are known in the field of fluorescent oligonucleotide and protein microarrays, may be applied to the microarray image files generated by MSI. Specifically, various methods of signal normalization, some of which are reviewed in (Quackenbush Nat Genet 2002) and (Bilban et al. Curr Issues Mol Biol 2002) may be utilized. Additionally, methods of finding locations of individual spots on a microarray, commonly known as gridding or addressing, and methods of separation of foreground intensities from background intensities, commonly known as segmentation, may be utilized.

Microarray images generated by MSI belong to the group of multivariate images. Accordingly, known methods of statistical and image analysis, which are commonly known as Multivariate Analysis, Multivariate Statistical Analysis or Multivariate Image Analysis, may be applied to the microarray MSI datasets. For example, such methods may include, but are not limited to, Principal Component Analysis, Multivariate Regression Analysis, Redundancy Analysis and Cluster Analysis.

Some of the above methods have been previously applied to the analysis of tissue and tissue microarray images generated by MALDI MSI, however they have not been applied to the analysis of biological microarrays, in particular random microarrays. Methods of multivariate analysis are described in numerous publications, for example Barbara G. Tabachnick, Linda S. Fidell “Using Multivariate Statistics” (5th Edition) Allyn & Bacon, Inc. Needham Heights, Mass., USA ©2006 ISBN:0205459382 and Sam Kash Kachigan “Multivariate Statistical Analysis: A Conceptual Introduction” (2^{nd} Edition) Radius Press; ©1991 ISBN-10: 0942154916.

Also, as described in greater detail below, in an embodiment, the microarray image data generated by MSI may be analyzed by a group of statistical analysis methods, which are commonly known as Exploratory Data Analysis and Confirmatory Data Analysis, and similar techniques.

Visualization of the Microarrav MSI Data

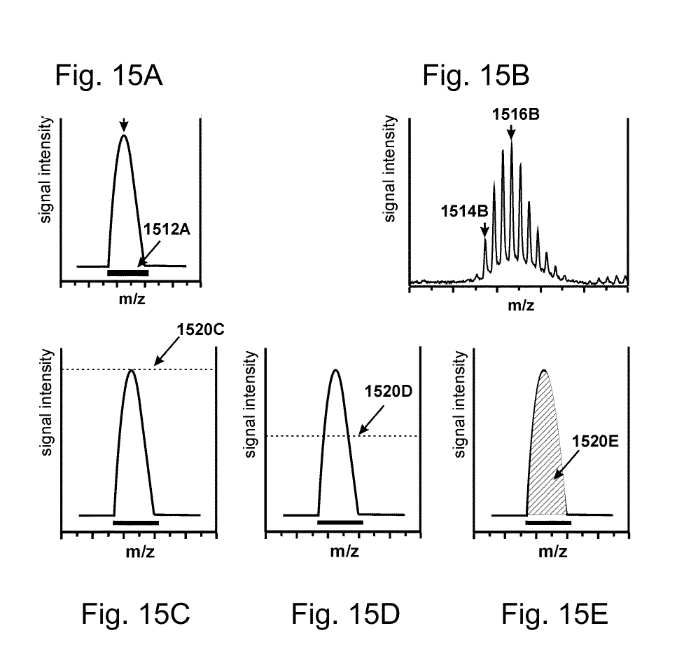

Datasets generated by microarray MSI may be used to create a series of microarray images, which will provide detailed information about the microarray morphology including mapping of analyte-containing spots, assessment of the analyte distribution within individual spots and determination of the size, shape and degree of overlap for individual spots. The existing image analysis software, for example BioMap or Bruker flexImaging™ may be used to produce pseudo-color or monochrome microarray images. The microarray images usually reveal microarray areas, in which the signal intensity measured in a specific mass channel (m/z) is above a certain threshold. In order to produce a single mass channel image, the signal intensity threshold and the appropriate mass channel must be selected. The signal intensity threshold may be usually selected to be above the background (noise) level. FIGS. 15A-15E show several non- limiting options, which may be used for a mass channel selection. In addition to a single mass channel 1510A, a continuous mass range comprising several individual mass channels 1512A may be selected for visualization, as shown in FIG. 15A. For example, in the time-of-flight instruments, a continuous mass range selection may be used to compensate for small variations in the measured molecular weight of analytes caused for example, by the microarray slide tilting, variations in the slide dimensions or variations in the thickness of matrix layer. For peaks that exhibit isotope distribution, a monoisotopic peak 1514B or the most intense peak 1516B may be selected, as shown in FIG. 15B. When a continuous mass range is selected, the signal associated with the mass range may be calculated as the maximum intensity 1520C, as shown in FIG. 15c, mean intensity 1520D, as shown in FIG. 15D, or area under the peak 1520E, as shown in FIG. 15E. Also, microarray images may be created using the total ion current, which represents the combined signal across the entire spectral range. Microarray visualization using the total ion current may be used to identify individual microarray spots regardless of the type of analyte.

An alternative visualization method is also possible, in which a combination of several discontinuous mass channels or mass ranges is used to create a single microarray image. The individual mass channel and mass range data may be obtained from a single or several different MSI scans of the microarray. Such method may be used, for example, to visualize distribution of a precursor protein after the trypsin digestion by using MSI data from mass channels, which correspond to individual digested fragments of the original precursor protein. The method may be also used to visualize distribution of a polypeptide analyte by using MSI data from mass channels, which correspond to the PSD fragments of the original polypeptide. The method may be also used to visualize distribution of a protein complex by using MSI data from mass channels, which correspond to the individual components of the original complex. In general, using data from multiple mass channels to visualize distribution of a specific analyte may help increase statistical confidence in the analyte identification, particularly when complex mixtures of analytes are present on a microarray. In an embodiment, microarray visualization using a combination of mass channels is performed based on the available information about the analyte sequence, analyte structure on individual beads and specific experimental protocols used to fabricate and image the microarray.

Various specific rules may be applied to the creation of microarray images using a combination of discontinuous mass channels or mass ranges. The total number of mass channels may be specified. The signal intensity threshold may be specified for each channel. The image visualization rules may require either all mass channels or a specific number of mass channels to have signal intensity above the threshold. In addition, the individual mass channels used to produce microarray images may be assigned specific weights.

The datasets generated by MALDI TOF MSI contain data in hundreds of thousands of individual mass channels, therefore a large number of independent microarray images may be produced, which correspond to a specific mass channel or a specific mass range. Such images may be displayed in individual windows providing a quick overview of distribution of specific analytes on a microarray. The ability to independently image multiple channels is a significant advantage over the more limited fluorescence readout.

Microarray Image Overlay

Microarray images created from single or multiple mass channels or mass ranges may be used to generate image overlays. Image overlay techniques are known in the image analysis applications including biomedical image analysis software such as BioMap. In the field of microarray data analysis, the image overlay may be used to provide qualitative and quantitative evidence for the co-localization of different analytes on a microarray. For example, images of analytes, which are present together on a microarray, are expected to have significant overlap, while images of unrelated analytes are expected to have little or no overlap. To perform an image overlay, at least two images must be supplied, each image comprising an array of pixels and intensity values associated with each pixel. Because of the large number of mass channels available in the microarray MSI datasets, the image overlay may be extended to more than 2 images recorded in different mass channels.

In addition to the standard two-image pseudo-color overlay, other alternative forms of image overlay may be envisioned, particularly for the overlay of multiple images. For example, logical operators such as AND, OR, XOR and NOT may be used depending on a specific microarray fabrication procedure and in accordance with the structure of analytes on beads. The signal intensity may be also considered in the image overlay. Some basic principles of image overlay, which are applicable to the microarray MSI data analysis, are described in R. Gonzalez and R. Woods Digital Image Processing Addison-Wesley Publishing Company, 1992.

The image overlay procedure may be performed using microarray data obtained from a single MSI scan. Alternatively, the image overlay may be performed using data obtained from several MSI scans of the same microarray area, including MSI scans performed using different MS instruments. Furthermore, the image overlay may be performed using data obtained from MSI and fluorescence, luminescence, autoradiography or SPR imaging of the same microarray area. Also, the image overlay may be performed using data obtained from MSI and visible scan of the same microarray area. The fluorescent, luminescent and visible images of a microarray may be used to perform microarray gridding (addressing) and segmentation according to the microarray data analysis methods.

A non-limiting example of using the microarray MSI image overlay procedure to confirm co-localization of two different analytes on a microarray is provided below. An array image in the first analyte-specific mass channel or a mass range is produced to visualize spots containing first analyte. A second array image in the second analyte- specific mass channel or a mass range is produced to visualize spots containing second analyte. The two images are superimposed using the image overlay procedure and spots containing both first and second analytes are visualized. It should be noted that each analyte spot may comprise several microarray pixels. The spot overlap may be calculated as the total number of pixels that exhibit above-threshold signal from both the first and second analytes divided by the total number of pixels that exhibit above-threshold signal from the first or second analytes. The two analytes are considered co-localized if their spot overlap is at least 25%, preferably at least 50%, more preferably at least 75% and most preferably, at least 90%. The spot overlay procedure may be extended to three, four or a greater number of analytes. Conversely, the image overlay procedure may be used to provide a quantitative measure of the absence of analyte co-localization. The two analytes are considered to be present separately on a microarray if their spot overlap is less than 50%, preferably less than 25%, more preferably less than 10% and most preferably less than 1%.

Statistical Analysis of the Microarray MSI Data

Various statistical methods may be used for the analysis of microarray MSI datasets. For example, there exist several different levels, on which the microarray data may be statistically analyzed. First, each pixel on a microarray may comprise multiple single-shot mass spectra, which are usually averaged to produce the final spectrum. Although the signal intensity may vary significantly between individual single-shot mass spectra, monitoring the signal intensity may be used to estimate the extent of analyte depletion within a particular spot. Second, each analyte spot may comprise several pixels depending on the image resolution. The distribution of intensity for each pixel within the analyte spot may be uniform or have a specific pattern, for example a radial gradient. Third, the microarray may contain several replicate spots for each type of analyte, for example if the bead library used to produce the microarray contains multiple identical beads. Furthermore, the microarray data may be also statistically analyzed at the level of individual mass spectra, for example using multiple mass channels or mass ranges for a particular analyte.

There exist various statistical methods, which may be applied in the microarray MSI format, including methods of descriptive statistics, statistical inference, correlation and regression analysis, multivariate statistics and others. For example, descriptive statistics may be used to analyze replicate spots in a microarray. For each set of replicate spots, the total number of spots with identical analyte, the number of pixels in each spot, mean, median, standard deviation, minimum and maximum signal may be measured.

Statistical analysis of the microarray MSI data may be used to perform quality control of the microarray preparation. For example, the size of individual spots (pixel count), distribution of signal intensity within individual spots and distribution of signal intensity between different spots may be measured. The statistical data may be also used to perform identification of individual spots on the microarray.

Statistical analysis of the microarray MSI data may be also used to detect the presence of both known and unknown analytes and the microarray data analysis may be performed in both confirmation and discovery modes.

Furthermore, statistical analysis of the microarray MSI data may be used to establish qualitative and quantitative relationships between different analytes on a microarray for the purpose of detecting interactions between those analytes. The statistical analysis may include methods that belong to the category of Statistical Hypothesis Testing, Exploratory Data Analysis and Confirmatory Data Analysis as well as others. The use of two basic tools of statistical analysis, Histograms and Scatter Plots in the microarray MSI format is described here.

Histograms: Histograms, which are graphical representations of distribution of data, may be generated for microarray images created as described above. The X-axis of a microarray histogram represents bins of signal intensities for a specific mass channel and the Y-axis represents the frequency, with which the signal in that intensity range appears on the microarray. Effectively, the Y-axis represents the number of microarray pixels that exhibit a signal of specific intensity. Histograms may be used for a variety of applications, for example to determine the foreground and background signal intensity during the process of microarray segmentation. Variations of standard histograms, such as log-histograms and multidimensional histograms may be also created.

Scatter Plots: Scatter plots show relationships between two or more variables and may be used to analyze multiple parameters in the microarray MSI data format. The data used in scatter plots may be generated by a single MSI microarray scan, different MSI scans of a same microarray, MSI scans of different microarrays, or a combination of scans by MSI and other techniques, e.g. fluorescence. In addition to two-dimensional linear scatter plots, logarithmic and multidimensional scatter plots may be constructed.

In one example, the scatter plot variables are signal intensity measured in a first mass channel profiled against signal intensity measured in a second mass channel for individual pixels within the same microarray. Such scatter plot may be used to detect co-localization of two or more analytes measured in different mass channels. There is provided an example of using a scatter plot to detect co-localization of two analytes with each analyte measured in a specific mass channel. In this example, the two analytes are present in multiple locations throughout the array resulting in multiple points in the scatter plot. Points on the scatter plot, which have positive intensity in both coordinates, represent pixels where the signal from both mass channels is detected. Points that have positive intensity in only one coordinate (i.e. points located on one of the axes) represent pixels where the signal from only one of the mass channels is detected. Thus, the two analytes are co-localized if the points on the scatter plot have positive intensity in both coordinates.

The scatter plot analysis may be also used to obtain quantitative information about distribution of analytes on the microarray, due to the fact that the ratio of signal intensities for two analytes, which are present in a same spot, is correlated with the relative amounts of these analytes in that spot. Therefore, while the signal intensity for two analytes may vary throughout the microarray, the intensity ratio should remain similar. The scatter plot analysis may also include the best fit or trendline analysis to determine whether the profiled variables can be described by a linear or nonlinear regression.

Using the Microarray MSI Data in Biological Applications

The present disclosure enables various applications of biological significance including, but not limited to, the following: (1) detection of interaction between multiple analytes in a microarray format; (2) quantitative detection of multiple analytes in a microarray format and (3) detection of analyte modifications in a microarray format.

Detection of Interaction Between Analytes

In an embodiment, the present disclosure provides methods of interaction profiling using mass spectrometry imaging in a microarray format. In an embodiment, molecular weights of the analytes present on a microarray are known. An analyte may be a molecular complex, an intact molecule, a molecular fragment, or molecules serving as labels or mass tags. Each analyte may be identified using one or several mass channels or mass ranges. The microarray data, which is used to detect interaction between analytes, may be obtained from one or several different MSI scans of a microarray. The different MSI scans may be recorded in a different mass range, using different spectral resolution or different measurement mode, for example linear and reflector or MS TOF and MS TOF-TOF.

In an embodiment, co-localization of analytes in a microarray spot is used to establish or confirm their interaction. The procedure to determine whether individual analytes are co-localized on a microarray is described in previous sections. For microarrays, which are produced from bead libraries, co-localization of specific analytes on a microarray may be used to establish their interaction on a particular bead within the bead library. For example, binding of a target molecule to a capture reagent or binding of a probe molecule to a target may be measured. The analyte co-localization procedure may be also used to perform assignment of individual components within a protein complex. Additionally, the procedure described here may be used to assign multiple enzymatically digested peptide fragments to their original precursor protein.

In an embodiment, microarray MSI data may be also used to determine specificity of interaction within a group comprising multiple capture reagents and multiple targets (interaction profiling). For example, microarray spots that contain a specific capture reagent are identified and microarray spots that contain a specific target analyte are identified. The overlap between the two images is measured and the substantial spot overlap serves as a measure of interaction. The spot overlap is preferably greater than 25%, more preferably greater than 50% and most preferably greater than 75%. The above procedure may be performed for every type of capture reagent and every type of target analyte present on a microarray. It is possible that multiple capture reagents will interact with a single target and vice versa multiple targets will interact with a single capture reagent. Therefore, microarray MSI data may be used to measure the total number of distinct target analytes that interact with a single capture reagent and vice versa, the total number of distinct capture reagents that interact with a single target analyte. For example, this procedure may be used to assess specificity of antibody-antigen interactions.

In an embodiment, microarray MSI data may be used to confirm the absence of interaction between a specific target analyte and capture reagent. In this example, the absence of spot overlap serves as the measure of the absence of interaction. The degree of spot overlap may be less than 50%, preferably less than 25%, more preferably less than 10% and most preferably less than 1%.

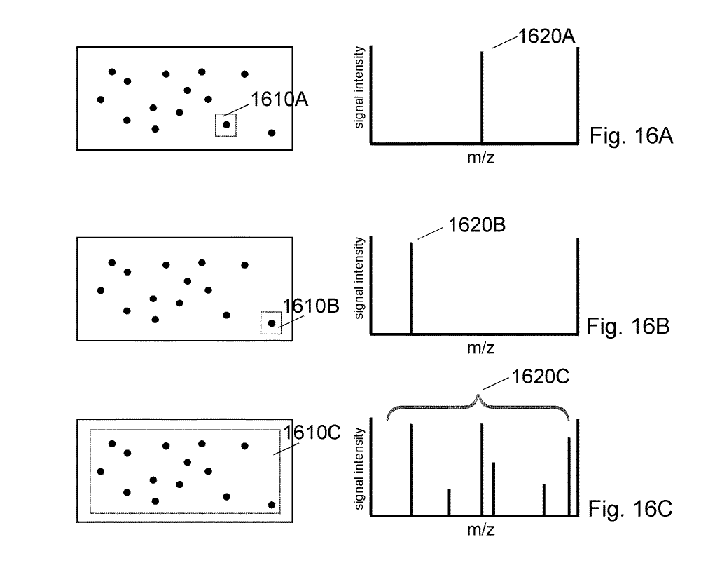

In an embodiment, microarray MSI data may be used to perform global microarray analysis. In this approach, instead of analysis of individual pixels, the spectral data from multiple pixels within the microarray, up to the entire microarray area, is co- added and analyzed. FIG. 16 demonstrates the principle of a global microarray analysis. While MSI data from individual pixels 1610A and 1610B shows only signals 1620A and 1620B that arise from analytes present in that specific area, as illustrated in FIG. 16A and FIG. 16B, respectively, the data from multiple pixels 1610C within the microarray in FIG. 16C shows combined signal 1620C from all analytes present on a microarray within that area. The spectral co-addition procedure for analysis of data from multiple pixels is known for the biological tissue MS imaging, but not for the microarray MSI. The global microarray analysis may be used, for example to: (i) establish that a specific target analyte is present on a microarray, for example that it binds to any member in a bead library, which was used to produce the microarray. In this example, the presence of signal due to target analyte indicates that the target interacts with at least one type of capture reagent; (ii) determine how many distinct targets bind to the specific microarray, which may comprise all of the capture reagents present on all of the beads within the bead library used to produce the microarray; (iii) conversely, establish the absence of interaction between the target of interest and the microarray, which may include any of the capture reagents on any member of the bead library, used to produce the microarray. The absence of signal due to the specific target analyte indicates that the target does not interact with any member on a microarray. The disclosed application may be also utilized in drug development studies to probe interaction of a small molecule (drug candidate) with its potential targets. The global microarray analysis may be also used to perform quality control check of a microarray fabrication and imaging processes.

The procedures used to establish interaction between analytes may additionally utilize the fact that there exist a known number of replicates for each analyte. For example, if the microarray is produced from a bead library, the number of replicate spots on the microarray should be fewer than or equal to the number of replicate beads in the bead library. If the analytes are transferred from beads onto a microarray with 100% efficiency, the number or replicate spots is equal to the number of replicate beads. However, because some beads may be lost during the microarray fabrication and analytes may not be transferred efficiently from some beads, the number of replicate spots may be smaller than the number of corresponding replicate beads. For each type of analyte, the number of replicate spots on a microarray relative to the number of replicate beads in a bead library is preferably over 50%, more preferably over 75%, and most preferably over 90%. In one example, to determine the fact of interaction between a specific capture reagent and a target analyte, the known number of replicates for different capture reagents is compared to the experimentally determined number of spots for a specific target analyte. In this approach, various statistical methods may be used to distinguish the fact of true interaction from a random overlap.

In general, the presence of multiple replicates for each analyte and possibly multiple pixels within each spot will allow various methods of statistical analysis to be applied to the analysis of microarray data to determine interaction between different analytes and to compensate for possible variations in the microarray fabrication and readout procedures. Variations in the microarray fabrication and readout may occur for example, during the binding of analytes on beads, transfer of analytes from beads onto a microarray, application of MALDI matrix to the microarray slide and MS measurements. Statistical methods, which are applied to the analysis of replicate data, may belong to the categories of Hypothesis Testing, Discovery Data Analysis or Confirmatory Data Analysis.

In an embodiment, the present disclosure provides two alternative methods of the mass spectrometric microarray data analysis that may be used to establish co-localization of analytes on the microarray, for example, in interaction profiling studies. In an embodiment, the first method involves overlay of individual analytes' spots using images generated in analyte-specific mass channels. This method may require using the microarray image data set, e.g. mass spectra and their associated coordinates. In an embodiment, the alternative method involves using methods of statistical analysis, such as scatter plot analysis, to directly compare intensity of peaks arising from specific analytes for multiple mass spectra recorded within the microarray. In the latter approach the location of a specific mass spectrum on the microarray is not important and the microarray data set may be supplied simply as a collection of individual mass spectra without their associated coordinates. Furthermore, the latter approach does not distinguish between mass spectra collected from different pixels within the same analyte spot and mass spectra collected from different analyte spots. The comparison of spot overlay and scatter plot procedures to determine interaction between different analytes is presented in Example 24.

Detection of Interaction with Unknown Target Analytes

The analysis of mass spectrometric microarray data may be also performed to determine interaction between the capture and target in the case when the identity of target analytes, such as the molecular structure, sequence or even molecular weight, is not known. One example is interaction between a combinatorial peptide library with multiple affinity reagents and a complex biological sample, such as a tissue extract or a biological fluid.

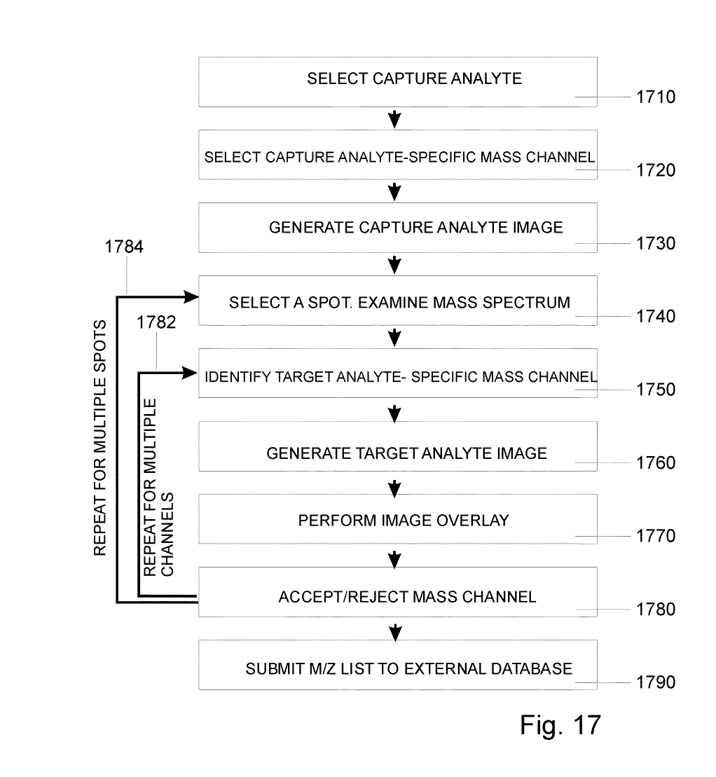

FIG. 17 schematically represents an embodiment process, which may be used to detect interaction between a known capture analyte and an unknown target. First, a capture analyte is selected I step 1710 and a mass channel or a mass range, which is specific for the particular capture analyte, is selected in step 1720. Then, distribution of that particular capture analyte on a microarray is analyzed in step 1730, for example, by generating a microarray image. Mass spectra in the microarray spots, where the capture analyte is found in sep 1740, are searched for the presence of additional peaks that do not belong to the capture analyte in step 1750. The distribution of signal for these mass channels on the microarray is also analyzed in step 1760. Previously disclosed methods, such as image overlay, are applied in step 1770 to determine whether correlation between the signal from capture analyte and signal in the newly found mass channels is statistically significant in step 1780. The above procedure generates a list of mass channels (m/z values) associated with each particular capture analyte in step 1782 and step 1784. The resulting m/z list may be submitted to external databases, e.g. MASCOT, to determine identity of the target analyte in step 1790. In the case of protein or polypeptides, the identity of target analytes may include the protein sequence and presence of post-translational modifications and mutations. Identification of the unknown analyte may be also performed without the microarray image analysis, for example by using the scatter plot method.

Various additional information may be available that will facilitate interpretation of the acquired data. This may include description of methods used to generate or read the microarray, for example, the use of enzymatic digestion, possible analyte fragmentation due to PSD or CID mechanisms and the relative error of the molecular weight measurement. In the case of digestion with trypsin and similar enzymes, the disclosed workflow represents a variation of experimental approach known as peptide mass fingerprinting (PMF). The PMF approach has not been previously applied in a microarray format.

Furthermore, optical data may be also used to determine identity of the target analytes, for example if target molecules react with a target-specific fluorescent antibody.

Various mathematical methods including methods of Exploratory Data Analysis and Confirmatory Data Analysis may be applied to the procedures described here. For example, some of the applicable methods are reviewed in the NIST/SEMATECH e-Handbook of Statistical Methods, (http://www.itl.nist.gov/div898/handbook/, accessed 03.19.2011).

Quantitative Analyte Detection Using the Microarray MSI Data

In an embodiment, the present disclosure enables quantitative analysis by mass spectrometry in a microarray format. For microarrays fabricated from bead libraries, the presently disclosed embodiments enable quantitative measurements of analytes originally present on beads. In general, the disclosed methods for measuring the analyte concentration are based on the analysis of signal intensity for mass channels, which are associated with specific analytes. Prior to performing the analysis known methods of mass spectrometric and microarray data processing, such as baseline correction and signal normalization may be applied to the microarray datasets. Additionally, methods of finding locations of individual spots on a microarray, commonly known as gridding or addressing, and methods of separation of foreground intensities from background intensities, commonly known as segmentation, may be applied to the microarray datasets.

Various quantitative detection methods by mass spectrometry, in particular laser desorption-ionization MS, may be implemented in the microarray format including microarrays produced by transfer of analytes from bead libraries according to methods of the present disclosure. Both label-free (absolute quantitation) and label-based techniques may be implemented. With respect to proteomic measurements, exemplary methods that can be utilized in the microarray format are reviewed in (Elliott et al. J Mass Spectrom 2009) and (Brun et al. J Proteomics 2009). Examples of quantitation methods that may be used in the microarray format include, but are not limited to, iMALDI, SISCAPA, ICAT, iTRAQ, SILAC, AQUA, QconCAT and PSAQ. Examples of absolute quantitation methods are multiple reaction monitoring (MRM) methods and other techniques measuring the ion current. In addition to methods based solely on mass spectrometry, hybrid MS-fluorescence measurements of a microarray are also possible. The use of fluorescence for quantitative detection of analytes in the microarray format has been well documented.

By way of a non-limiting example, a description of several different methods of analyte quantitation by MS in a microarray format is provided here. The disclosed methods use an example of analytes, which are transferred from bead libraries and measured on a microarray. The disclosed methods are compatible with various methods of microarray fabrication from bead libraries including use of digestive enzymes, exposure to low pH medium and photoelution. Alternative implementations and modifications of the described procedures will be apparent to a person skilled in the art. Some of the described quantitation methods require measurement of at least two distinct analytes from the same area of a microarray and therefore may benefit from the previously disclosed methods, which establish co-localization of analytes.

The disclosed analyte quantitation methods allow collection of large amounts of experimental data, which enables application of powerful methods of statistical analysis to be performed on the microarray MS datasets resulting in greater confidence of quantitative measurements. For example, the signal from a specific analyte on a microarray may be measured and analyzed from: (1) multiple single-shot spectra collected from a single microarray pixel; (2) multiple pixels measured within a microarray spot and (3) multiple replicate spots with the same analyte measured throughout the microarray.

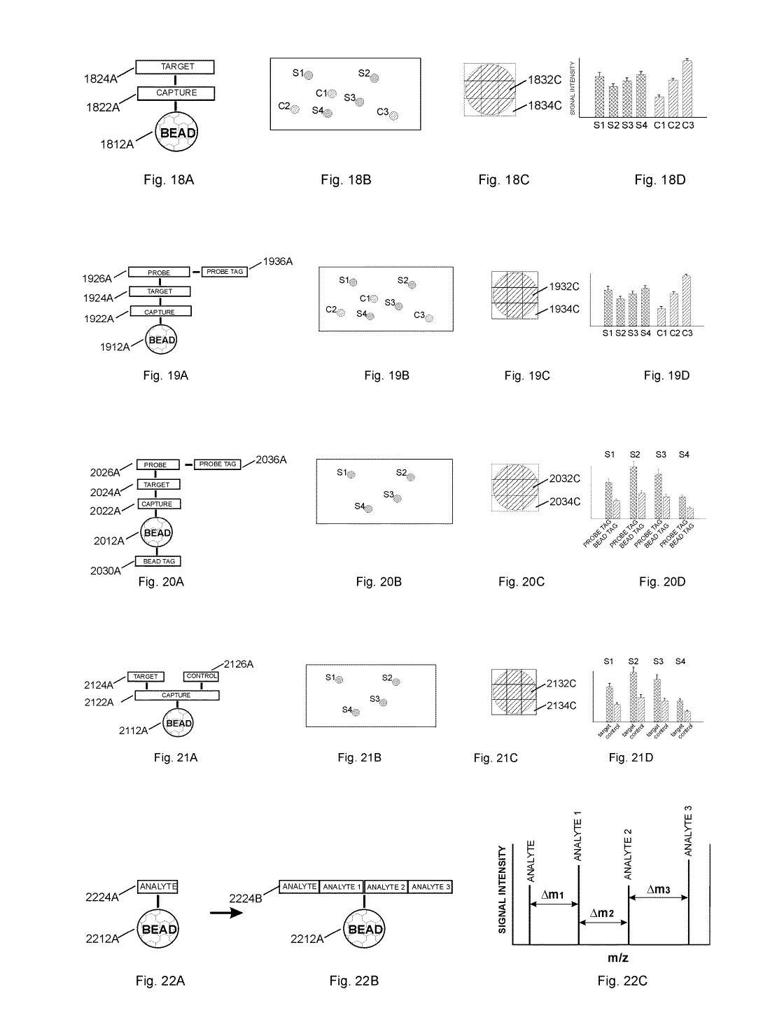

FIGS. 18A-18D shows a schematic description of an embodiment quantitative measurement method, which allows label-free detection of target analytes. FIG. 18A is a schematic representation of a bead design showing elements sufficient to perform quantitative measurement. The bead 1812A is conjugated to a capture molecule or molecular complex 1822A, which is bound to the target molecule or molecular complex 1824A. In this example, only the target analyte 1824A is used for quantitative measurements. Note that additional elements according to FIG. 11 (e.g., a probe, a bead label, a target label) may be also present on beads. Some of these additional elements may be used for the analyte identification. There may be several replicate beads for each type of target, preferably between 2 and 10,000, more preferably between 10 and 1000. The target or its molecular fragment is transferred onto an array of microspots. If there are replicate beads in the bead library, replicate spots are formed on a microarray (FIG. 18B, labels “S1”, “S2”, “S3”, “S4”). In addition, control spots with a known amount of analyte may be provided on a microarray (FIG. 18B, labels “C1”, “C2”, “C3”). The analyte in control spots may be structurally identical or similar to the measured target analyte. The control spots may be produced by transferring analyte from control beads provided in the bead library, or by depositing a known amount of analyte in specific areas of the microarray. The analyte in control spots may be distinguished from the analyte in sample spots using: (i) positional encoding (i.e., the control analyte is deposited in specific areas of the microarray), (ii) internal labeling, e.g., isotope labeling of the analyte or (iii) external labeling, e.g. providing additional analytes serving as identification markers, such as bead labels (1130 in FIG. 11), which are transferred on a microarray along with the measured analytes. FIG. 18C shows that the microarray readout may comprise several pixels 1834C per analyte spot 1832C, in which case the combined signal intensity from all pixels within the spot is preferably measured as the analyte signal. FIG. 18D shows a schematic representation of measured signals from replicate spots containing the target (labels “S1”, “S2”, “S3”, “S4”) and optional control (labels “C1”, “C2”, “C3”) analytes. Data acquisition from multiple spots within the microarray enables statistical analysis including for example, measurements of the mean, median and standard deviation for each type of analyte. A further modification of this method involves providing control spots with different amounts of analyte, so that a calibration curve may be constructed for more accurate quantitative measurements.

FIGS. 19A-19D show a schematic description of an embodiment of a quantitative measurement method, which allows label-based detection of target analytes. This method may be used when direct measurement of target analytes is impractical, for example the analyte mass is outside of useful MW range of the instrument or the analyte molecules are unstable. FIG. 19A is a schematic representation of a bead design showing elements sufficient to perform quantitative measurements. The bead 1912A is conjugated to a capture molecule or molecular complex 1922A, which is bound to the target molecule or molecular complex 1924A, which is bound to the probe molecule or molecular complex 1926A, which contains a probe tag 1936A. In this example, the probe tag serves as the quantitative reporter molecule because the signal intensity of the probe tag is related to the concentration of the target analyte on bead. Alternatively, a target tag (1134 in FIG. 11) may serve as a quantitative reporter molecule in the approach similar to the ICAT method. It should be noted that additional elements according to FIG. 11 may be also present on beads and used for the analyte identification. There may be several replicate beads for each type of target, preferably between 2 and 10,000, more preferably between 10 and 1000. The probe tag 1936A or its molecular fragment is transferred onto a microarray and measured quantitatively. If there are replicate beads in the bead library, replicate spots are formed on a microarray, as shown schematically in FIG. 19B with labels “S1”, “S2”, “S3”, “S4”. In addition, control spots with a known amount of analyte may be provided on a microarray as shown in FIG. 19B by labels “C1”, “C2”, “C3.” The probe label may be identical throughout the microarray or specific for each type of target analyte. In the former case, an additional analyte serving as an identification label for the target analyte, e.g., the target molecule itself, or the bead label is also provided on a microarray. FIG. 19C shows that the microarray readout may comprise several pixels 1934C per analyte spot 1932C, in which case the combined signal intensity from all pixels within the spot is preferably measured as the analyte signal. FIG. 19D shows schematic representation of measured signals from the target (labels “S1”, “S2”, “S3”, “S4”) and optional control (labels “C1”, “C2”, “C3”) analytes. Data acquisition from multiple spots within the microarray enables statistical analysis including for example, measurements of the mean, median and standard deviation for each type of analyte. This method may further utilize control spots with different amounts of analytes, as showin in FIG. 19D with labels “C1”, “C2”, “C3”, so that a calibration curve may be constructed for more accurate quantitative measurements.

FIGS. 20A-20D show a schematic description of an embodiment quantitative measurement method, which allows label-based detection of target analytes that involves at least two analytes measured quantitatively per sample. FIG. 20A is a schematic representation of a bead design showing elements sufficient to perform quantitative measurement. The bead 2012A is conjugated to a capture molecule or molecular complex 2022A, which is bound to the target molecule or molecular complex 2024A, which in turn is bound to the probe molecule or molecular complex 2026A, which contains a probe tag 2036A. Additionally, the bead is conjugated to a bead tag 2030A. The amount of analyte conjugated to beads as the bead tag 2030A is preferably known. In this example, the probe tag 2036A serves as the quantitative reporter molecule because the signal intensity of the probe tag is related to the concentration of the target analyte on bead. The bead tag 2030A analyte serves as reference quantitative molecule. Alternatively, a target tag (1134 in FIG. 11) may serve as a quantitative reporter molecule instead of the probe tag. Note that additional elements according to FIG. 11 may be also present on beads and used for the analyte identification. There may be several replicate beads for each type of target, preferably between 2 and 10,000, more preferably between 10 and 1000. The probe tag 2036A or its molecular fragment and the bead tag 2030A or its molecular fragment are transferred onto a microarray and measured quantitatively. If there are replicate beads in the bead library, replicate spots are formed on a microarray, as shown schematically in FIG. 20B (labels “S1”, “S2”, “S3” and “S4”). The probe tag may be identical or specific for each type of target analyte. Similarly, the bead tag may be identical or specific for each type of target analyte. If the bead tags are specific for each type of target analytes, they may additionally serve as identification labels. In this example, because the two analytes 2030A and 2036A are measured together, the signal may be collected and analyzed from each pixel 2034C within the microarray as well as measured from an entire analyte spot 2032C, which may contain several pixels, as shown schematically in FIG. 20C. The signal intensity may be used for quantitative measurement of the amount of target analytes, for example by comparing the ratio of peaks due to the probe tag 2036A and bead tag 2030A that may be collected from multiple spots, as illustrated in FIG. 20D. Various amounts of control analytes may be additionally provided to construct calibration curves. Furthermore, experimental relationships between the signal intensity due to the reference analyte and the amount of analyte on beads may be known.

FIGS. 21A-21D is a schematic description of an embodiment quantitative measurement method. In this approach, a measured amount of control, or reference analyte is added to the medium containing target analyte before the target analyte is bound to the capture reagent. Both target and control analytes preferably have similar chemical structure and therefore similar affinity for the capture reagent. The target and control analytes are then purified together. One example of this approach is Stable Isotope Labeling with Amino acids in Cell culture (SILAC) method, in which the control analyte is the heavy isotope-labeled version of a target analyte. Another example is proteolytic peptides generated by methods known as iMALDI and SISCAPA. Another example is two peptides with the same antibody affinity tag. FIG. 21A is a schematic representation of a bead design showing elements sufficient to perform quantitative measurement. The bead 2112A is conjugated to a capture molecule or molecular complex 2122A, which is bound to a mixture of target molecule or molecular complex 2124A and control analyte 2126A. Note that additional elements according to FIG. 11 may be also present on beads. These additional elements may be used for the additional analyte identification. There may be several replicate beads for each type of target, preferably between 2 and 10,000, more preferably between 10 and 1000. The target and control analytes or their molecular fragments are transferred onto a microarray and the ratio of signal due to these analytes is measured quantitatively. If there are replicate beads in the bead library, replicate spots are formed on a microarray, as illustrated schematically in FIG. 21B with replicate spots labeled “S1”, “S2”, “S3” and “S4”. In this example, because the two analytes are measured together, the signal may be collected and analyzed from each pixel 2134C within the microarray as well as measured from an entire analyte spot 2132C, which may contain several pixels, as depicted in FIG. 21C. Because the amount of control analyte is known, the intensity ratio of peaks due to the analytes 2124A and 2126A may be used for quantitative assessment of the amount of target analytes, as depicted in FIG. 21D. Such methods are known in the quantitative proteomics field but have not been performed in a microarray format.

Measuring Analyte Modification in a Microarray Format

The presently disclosed embodiments provide methods for measuring analyte modification by MSI in a microarray format. Analyte modification may occur, for example from a chemical reaction between the analyte and an enzyme, such as a protein kinase or phosphatase, which alters the analyte chemical structure. The modification reaction may either increase or decrease the molecular weight of analyte. Furthermore, the modification reaction may result in generation of several new analyte species from a single analyte. Also the modification reaction may not proceed to its full extent, resulting in the presence of a mixture of original unreacted and newly formed analytes. In an embodiment, analytes are immobilized on beads during the modification reaction and subsequently transferred onto a microarray and measured by MSI.

FIGS. 22A, FIG. 22B and FIG. 22C schematically show the measurement of analyte modification in a microarray format. In an embodiment, the molecular weight of original unreacted analyte 2224A, which is conjugated to a microbead 2212A, as shown in FIG. 22A, is known and is used to identify the specific analyte. Additional labels, e.g. bead tags (1130 in FIG. 11) may be also provided for the purpose of analyte identification. In refernece to FIG. 22B, the analyte modification reaction may result in appearance of modified forms of the original analyte 2224B, which are labeled “analyte 1”, “analyte 2” and “analyte 3”. The microarray MSI data may be analyzed from individual pixels or alternatively the signals from pixels, which constitute an individual analyte spot, are combined and subsequently analyzed. The resulting mass spectra are analyzed for the presence of a peak due to the unreacted analyte and presence of additional peaks due to modified forms of the analyte, as shown in FIG. 22C. The analysis of mass spectra may be used to obtain detailed information including: (i) the nature of analyte modification determined by the observed mass difference, (ii) the extent of analyte modification reaction determined by the comparison of signal intensity for the unreacted and reacted forms of analyte and (iii) the total number of distinct species determined by the total number of distinct peaks found in the spectra. The experimental design may be extended to include time series, i.e. performing the modification reaction for a specific duration of time and measuring the extent of analyte modification as a function of time. This approach may be used to study the reaction kinetics and activity of specific enzymes.

Using Optical and MS Image Data for the Identification of Microbeads and Analytes Present on Microbeads

In an embodiment, the methods of the present disclosure use a combination of optical and mass spectrometric image data, which is acquired from a microarray system comprising an array of microspots and a congruent array of microbeads, to identify analytes present in individual microspots.

In an embodiment, in the methods of the present disclosure, the experimentally obtained data identifying analytes present in individual microspots is combined with the data related to the bead fabrication history or bead fabrication protocol in order to identify analytes present on individual microbeads.

The use of optical, e.g. fluorescence microarray data in addition to the mass spectrometric microarray data can significantly increase the analytical power of bead- based assays measured in the microarray format. The ability to perform independent optical and MS readout from the same bead-analyte construct greatly increases the number of options available for the design of a specific bead assay. Optical, e.g. fluorescence readout may be used to supplement the mass spectrometric readout in a case when MS detection is not possible or not optimal for a particular analyte, for example when the analyte molecular weight is outside of the m/z detection range, the analyte transfer to the gas phase is difficult or the analyte undergoes extensive fragmentation inside the mass spectrometer.

For example, beads may be conjugated both to a polypeptide and a larger protein, which is further conjugated to a protein-specific fluorescently labeled antibody. Using MALDI TOF MS in the reflector mode enables highly accurate detection of the polypeptide, but not the protein or the antibody, which are outside the instrument detection range in the reflector mode. On the other hand, the dual MS-fluorescence imaging readout enables detection of the polypeptide and also the fluorescent antibody and further enables co-registration of the two images in order to assign MS and fluorescence signals to a specific location within the array. Subsequently, the known specificity of the fluorescent antibody is used to establish the presence of the corresponding protein antigen on beads displaying the fluorescent signal.

In an embodiment, distinctive optical properties of microbeads or optical properties of analytes conjugated to the microbeads are used to recognize identical bead- analyte constructs, i.e. replicates, within an array. For example, identification of replicate spots based on their optical properties enables statistical analysis of mass spectrometric data recorded from such replicate spots.

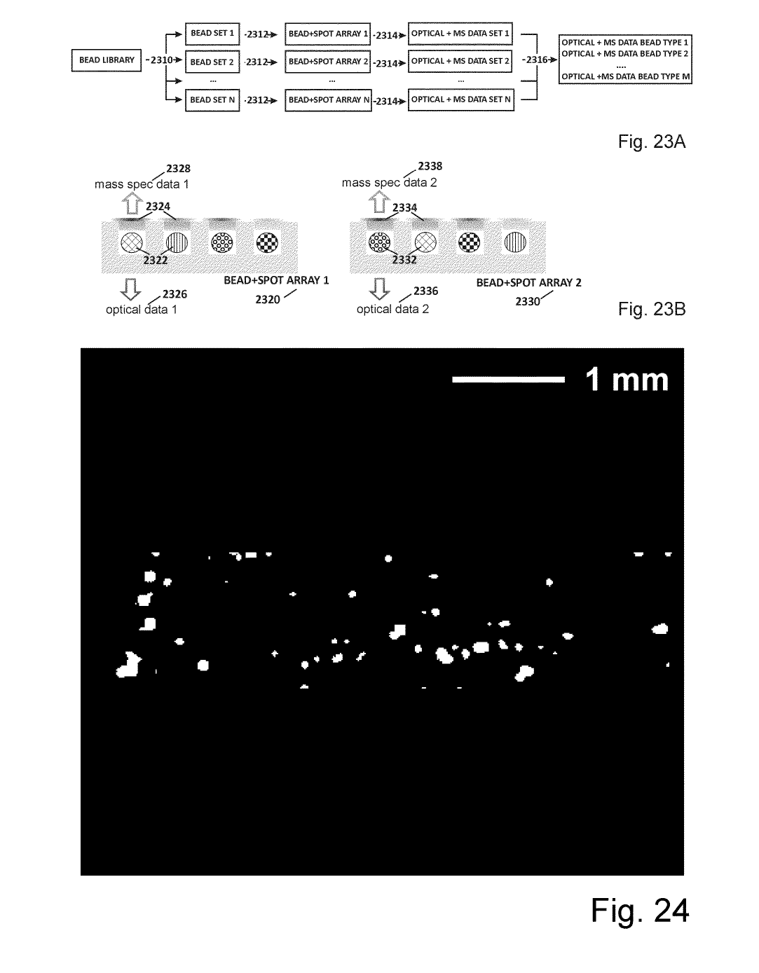

In an embodiment, distinctive optical properties of microbeads or optical properties of analytes conjugated to the microbeads are used to recognize identical bead- analyte constructs across several bead arrays. In this approach illustrated by a flow diagram in FIG. 23A, a bead library comprises multiple different bead types and multiple replicate beads, i.e. beads carrying identical analytes, for each bead type. The bead library is divided into two or more smaller bead sets as indicated by a group of arrows 2310 and each bead set is used to independently fabricate the combination of a bead array and a corresponding microspot array as indicated by a group of arrows 2312. The known methods of automated bead dispensing and optical bead sorting including flow cytometry may be used to ensure the presence of each of the different bead types in each of the bead sets. Alternatively the bead library may be divided into bead sets by automated or manual pipetting of bead suspensions. In the latter example statistical distribution of bead types within each bead set is expected, which may follow Poisson distribution.

Different conditions may be employed to release analytes from microbeads in each of the bead sets prepared by dividing the precursor bead library. For example, different digestive enzymes may be used. Furthermore, different parameters of the mass spectrometric data acquisition, e.g. different mass range, may be employed to measure each of the arrays of microspots fabricated from the bead sets as indicated by a group of arrows 2314. As a result, mass spectra acquired from identical beads located in different bead arrays may vary substantially.

Combining different sets of mass spectrometric data measured under different conditions for individual bead types as indicated by a group of arrows 2316 enables detailed analysis of the analyte structure that may not be possible to perform in a single MS experiment. This approach requires the ability to reliably identify bead-analyte constructs, including the bead-analyte constructs in different bead arrays, based on their signature optical (e.g., fluorescence) spectra, which is enabled by the methods of the present disclosure.

FIG. 23B schematically illustrates the disclosed method. A bead set 2322 comprising multiple bead types is used to fabricate an array 2320. A separate bead set 2332 containing beads identical to those in bead set 2322 is used to fabricate a separate array 2330. Identical beads are positioned in random locations throughout the arrays 2320 and 2330. The analyte release from bead sets 2322 and 2332 results in fabrication of groups of microspots 2324 and 2334, respectively. Depending upon the experimental conditions, analytes released from replicate beads may differ significantly between different arrays. The released analytes in each array are measured by mass spectrometry resulting in fabrication of mass spectrometric data sets 2328 and 2338. Different protocols of MS data acquisition employed to measure analytes in arrays 2320 and 2330 may further contribute to differences in MS data recorded from the replicate beads. The beads and bead-associated compounds within each array are also measured by optical imaging, for example fluorescence imaging, resulting in fabrication of optical data sets 2326 and 2336. The optical spectra may be recorded, for example, via fiber optic channels directly from beads submerged into microwells, in which case they remain largely independent of the used methods of analyte release and MS measurement. Therefore replicate beads will exhibit identical or very similar optical spectra while their mass spectra may differ substantially.

The disclosed approach may be used to measure a large variety of bead- conjugated molecular complexes comprising molecules of significantly different nature. For example, a molecular complex may be formed by a polypeptide and an oligonucleotide. Mass spectrometric detection of individual analytes within such complex may require using different ionization matrix, e.g. CHCA and 3-HPA and different ion mode (positive and negative, respectively).

Data Structure for Microarrays Fabricated from Bead Libraries

It is a feature of the present disclosure that sufficient amount of experimental data related to the methods of fabrication of microparticles, methods of fabrication of an array of microspots from the microparticles and properties of individual microparticles and microspots including possible optical properties is provided in the description of microarrays fabricated from bead libraries. Providing such data will facilitate mass spectrometric measurement of microarrays as well as downstream analysis of MS data.

The experimental data that may be provided for individual microarrays has been disclosed previously in the section titled “Microarray Data Available Prior to the Mass Spectrometric Analysis.” Such data may be supplied using numerous method of electronic data storage and data transfer known in the art including methods utilizing electronic databases. The data may be provided directly to the instrument control interface using known methods of electronic data transfer. Appropriate modifications of methods and algorithms controlling acquisition and analysis of MS data, which need to be made in order to accommodate the data structure of the present disclosure, are apparent to a person skilled in the art.

In embodiment, the methods of the present disclosure provide the description of individual analytes, which are present or may be present on a microarray fabricated from microparticles, in the form of m/z values associated with each analyte. This may be in addition to the commonly used forms of analyte description in the microarray format that may include common and systematic names of a compound, its chemical structure, chemical formula and molecular weight.

The rationale for providing a list of m/z values associated with a specific analyte is the fact that individual analytes initially present on the carrier microparticles may undergo substantial modification and fragmentation during the analyte transfer from the microparticles onto the solid support. For example, the analyte molecules may be split into smaller fragments when exposed to a digestive enzyme. The analytes may undergo additional fragmentation during the process of desorption-ionization via mechanisms known as PSD and neutral molecule loss. The analytes may even undergo additional fragmentation during the process of MS measurement via mechanisms known as CID and electron transfer dissociation (ETD). On the other hand, ionized analytes may be detected in various forms, such as molecular ions, dimers, trimers, multiply charged ions and adduct ions.

Because experimental conditions of analyte transfer, desorption-ionization from the solid support and MS detection may vary substantially between different experimental protocols, even identical compounds may give rise to dramatically different sets of signals in the mass spectra measured from different microarrays. One example is the use of different digestive enzymes utilized to achieve the analyte release from the microparticles.

Accordingly, it may be advantageous for a manufacturer or a supplier of the microparticles to provide a list of m/z values associated with each analyte or with each microparticle within a group of microparticles, e.g. a bead library. The list of m/z values may be specific for a particular assay, particular method of fabrication of the array of microspots, particular method of analyte desorption-ionization and particular method of MS data acquisition. The specific m/z values may be determined by in silico calculations using methods known in the art. Alternatively, the m/z values may be determined experimentally by performing a series of measurements under well-defined experimental conditions.

Providing the m/z data to the end user may be of substantial value as it greatly simplifies analysis of the generated MS datasets and also may serve as a quality control measure for various procedures performed prior to the MS data acquisition.

In general, the devices and methods of the instant disclosure provide, in an embodiment, an interface between the bead-based assay technologies and the mass spectrometry detection. Therefore, the devices and methods of the instant disclosure may be used in a vast variety of experimental applications, which demand high degree of multiplexing and the detailed analysis of the analyte including the label-free sample detection.

For example, the devices and methods of the instant disclosure may be used for analyzing peptide bead arrays for enzyme profiling. Individual peptides representing potential substrates for enzymes may be immobilized on beads generating bead libraries, which may be used to screen for a specific enzyme activity. After the reaction, the peptides attached by an acid-labile, base-labile or photolabile linker may be eluted from beads and their mass measured by mass spectrometry to identify modified peptides. This method is also suitable for quantitative analysis of the peptide modification reactions, which is achieved for example by comparing intensity of the unmodified and modified peptide mass-peaks. Furthermore, in the search of potential peptide-based enzyme inhibitors, the enzyme binding to the peptide may be also detected by mass spectrometry.

In an embodiment, the methods of the instant disclosure may be used in connection with peptide or peptidomimetic combinatorial libraries. Bead libraries containing hundreds of thousands of peptides or peptidomimetics serving as capture reagents can be screened against multiple target proteins to identify high affinity peptide- protein interactions. Both the capture reagent and the target can be identified by mass spectrometry, for example by photoreleasing the peptide or peptidomimetic from the bead and performing trypsin digestion of the protein. Alternatively, the protein binding can be detected by fluorescence. In addition to peptides and peptidomimetics, other compounds can be immobilized on beads, for example using methods of one-bead one-compound (OBOC) combinatorial library synthesis.

In an embodiment, the devices and methods of the instant disclosure may be utilized in connection with drug discovery studies. Proteins or protein complexes, which are potential drug targets, can be immobilized on beads and screened against various small molecules, which represent potential drug candidates, with the binding event detected by mass spectrometry, possibly using the multiple reaction monitoring. Note that the label-free detection provided by mass spectrometry is especially important in this case because introduction of a label would alter the molecular structure of the drug.

Screening for protein-protein interactions including those mediated by a ligand may also, in an embodiment, be performed using the devices and methods of the instant disclosure. The assay design is sufficiently flexible to simultaneously screen a library of proteins immobilized on beads against another library of proteins present in solution or in a complex biological medium. In addition, small molecules can be added to the mixture. The binding event is detected by analyzing the protein complexes on each bead, possibly using the protein mass fingerprinting method. This group of applications also includes the antibody screening and epitope mapping studies.

The devices and methods of the instant disclosure may be used in connection with the in-vitro evolution studies. In an embodiment, the devices and methods of the instant disclosure are used to analyze antibody arrays and antigen arrays. Compared to the conventional fluorescence-based methods where two antibodies with different specificity are required for each analyte (capture and detection), assays utilizing the detection by mass-spectrometry require only the capture antibody.

Biomarker discovery and validation studies are another application that may benefit from the devices and methods of the instant disclosure. The ability to analyze up to 500,000 samples or more on a single microchip is particularly attractive for the biomarker studies since the large number of different samples pooled together from many different sources can be analyzed in multiple replicates. One example of biomedical application, which may benefit from the presently disclosed embodiments, is serum- based diagnostics.

In an embodiment, the methods of the instant disclosure can interface with various microfluidic applications or emulsion-based methods. The disclosed devices and method provide an effective way to analyze contents of individual droplets, which are produced in a microfluidic apparatus, using mass spectrometry. A single bead with a specific capture reagent can be included in each droplet. Following the reaction, the beads are released from the droplets, transferred to the microarray plate and analyzed.

The devices and methods of the instant disclosure may be utilized for multiplexed purification of samples from a complex mixture, such as a biological medium for the purpose of mass spectrometry analysis. Various capture reagents with different specificity may be immobilized on beads and used to simultaneously concentrate and isolate multiple samples on beads. The examples of capture reagents are oligonucleotides for binding DNA and RNA molecules, proteins, peptides and peptoids for binding antibody molecules including antibodies of clinical and diagnostic importance, and antibodies and aptamers for binding protein and peptide molecules.

The devices and methods of the instant disclosure may be utilized under conditions that allow continuous or stepwise release of individual compounds from the microbeads. For example, different compounds may be conjugated to a single bead using two or more different types of photolabile linkers that are cleaved by light of different wavelength. Alternatively, the compounds may be bound to the bead surface and the bead interior, such beads commonly known as topologically segregated bilayer beads. Alternatively, the compounds may be only partially released within a specific time window, for example by utilizing slowly cleavable linkers. In this approach, the compound released from the beads using one release mechanism may be screened on the microwell plates by an appropriate assay, for example a cell viability assay, an optical assay or a mass spectrometric assay. Compounds released from the bead array in the first screening may then be depleted or removed from the microwell plate, for example by rinsing the plate, or by using desorption mechanism provided by ionization laser of the mass spectrometer and a second group of compounds sequentially released from the same bead array and analyzed by an appropriate assay, for example a mass spectrometric assay. The data obtained from such multiple measurements sequentially performed on the same bead array can be analyzed together using known techniques of image co-registration.

The devices and methods of the instant disclosure may be utilized by providers of mass spectrometric analytical services. For example, various analytical and reference labs, as well as proteomics and other core facilities that have mass spectrometers capable of high-resolution imaging may perform analysis of large bead libraries by first converting them into planar arrays of analytes and measuring the fabricated arrays by MS.

In an embodiment, a method of transfer of analytes from microparticles onto a solid support comprises providing a plurality of microparticles with bound analytes wherein the microparticles are positioned on a solid support and spatially separated, releasing the analytes from the microparticles, and localizing the released analytes in spots whereby dimensions of the spots containing the released analytes are similar to dimensions of the respective microparticles. In an embodiment, the released analytes are unambiguously identified with their respective microparticles. In an embodiment, the released analytes are detectable by mass spectrometry. In an embodiment, the method of mass spectrometry is selected from a group comprising Matrix Assisted Laser Desorption-Ionization, Desorption Electrospray Ionization, Desorption-Ionization on Silicon, Nanostructured Laser Desorption Ionization and Secondary Ion Mass Spectrometry. In an embodiment, the mass spectrometry is imaging mass spectrometry. In an embodiment, molecules that are not released from the microparticles are undetectable by mass spectrometry. In an embodiment, the analytes are selected from a group comprising a peptide, a peptidomimetic, a protein, a nucleic acid, a lipid, a carbohydrate, a small molecule and their combinations. In an embodiment, the analytes are complexes comprising at least two distinct molecules. In an embodiment, wherein the released analytes are molecular fragments. In an embodiment, wherein the microparticles with bound analytes are fabricated by bead-based or solution-based emulsion reactions. In an embodiment, wherein the microparticles are microbeads. In an embodiment, wherein the microbeads are monodisperse. In an embodiment, wherein diameter of the microbeads is between 250 nm and 1000 micron. In an embodiment, the solid support is a microwell array plate. In an embodiment, diameter of individual spots containing the released analytes is less than 2-fold of the diameter of individual microwells. In an embodiment, wherein the analyte release method is selected from a group comprising an exposure to electromagnetic radiation, an exposure to heat, a change of pH, a change of solvent, a change in concentration of an affinity ligand and an exposure to a digestive compound. In an embodiment, distinct analytes released from a same microparticle are co-localized on the solid support. In an embodiment, the transfer of analytes from the microparticles onto the solid support is performed quantitatively. In an embodiment, the released analytes are accumulated near surface of the solid support. In an embodiment, the transfer of analytes from the microparticles onto the solid support is used in bead- based analytical assays.

In an embodiment, a method of fabricating arrays suitable for analysis by mass spectrometry and optical spectroscopy comprises providing a solid support having a plurality of analytical sites wherein the solid support is compatible with mass spectrometry and optical detection, arraying a plurality of microparticles with bound analytes on the solid support whereby each analytical site contains no more than one microparticle, releasing the analytes from the array of microparticles whereby the released analytes are localized near their respective microparticles. In an embodiment, the solid support enables acquisition of mass spectra and optical spectra from individual analytical sites. In an embodiment, the fabricated array is compatible with mass spectrometry and optical detection performed in the imaging mode. In an embodiment, the method further enables acquisition of optical spectra before and after the analyte release. In an embodiment, the method further enables acquisition of optical spectra from the microparticles and separately from the released analytes. In an embodiment, the optical spectroscopy is fluorescence spectroscopy or luminescence spectroscopy. In an embodiment, the solid support is a fiber optic microwell array plate. In an embodiment, wherein the microparticles are optically encoded. In an embodiment, the microparticles comprise an array of microbeads and the released analytes comprise an array of microspots and the two arrays are spatially related. In an embodiment, the released analytes enable identification of compounds bound to the microparticles. In an embodiment, the compounds are affinity probes or enzyme substrates. In an embodiment, the optical spectra can be used to determine occurrence of an affinity binding event.

In an embodiment, a method of fabricating an array of analytes using a microfluidic device comprises providing a flow cell comprising at least a microwell array plate and a plurality of reagent-conjugated microparticles at least partially submerged into microwells wherein no more than one such microparticle occupies a single microwell, introducing at least one sample into the flow cell, allowing each sample to react with the reagents conjugated to the microparticles, and releasing analytes from the microparticles wherein the analytes are selected from compounds bound to the microparticles whereby the released analytes are identified with their respective microparticles and detectable by mass spectrometry. In an embodiment, the released analytes are selected from a group comprising unreacted reagents, reacted reagents, molecular fragments of the reagents, molecules bound to the reagents, fragments of molecules bound to the reagents and mass tags. In an embodiment, the microwell array plate further enables optical detection of reactions that occur between the sample introduced into the flow cell and the reagents conjugated to the microparticles. In an embodiment, the method further comprises the step of measuring the released analytes by mass spectrometry. In an embodiment, the method further comprises the step of comparing the optical and the mass spectra.

In an embodiment, a device for analysis of analyte-conjugated microparticles, the device comprises a solid support having a plurality of topological features of specific dimensions wherein the dimensions of topological features enable positioning of the microparticles at least partially inside the topological features whereby a majority of the topological features contain no more than one microparticle, wherein the microparticles positioned inside the topological features are accessible to analyte release agents wherein the release agents are selected from a group comprising chemical compositions in solid, liquid or gas form, heat and electromagnetic radiation and wherein the solid support restricts migration of analytes released from individual microparticles to vicinity of the respective microparticles. In an embodiment, the solid support further enables concentration of the analytes released from the microparticles. In an embodiment, the solid support further enables mass spectrometric detection of the analytes released from the microparticles. In an embodiment, the topological features are microwells or microchannles In an embodiment, the device further comprises a layer formed on surface of the solid support wherein the layer enables retention of liquids in surface areas surrounding openings into the topological features. In an embodiment, the layer is chemically non-reactive and electrically conductive. In an embodiment, surface area occupied by openings into the topological features comprises between 5% and 95% of total surface area. In an embodiment, specific distance between openings into the topological features is provided to minimize overlap between individual spots formed by the analytes released from the microparticles. In an embodiment, the topological features form an ordered grid or array. In an embodiment, the dimensions of topological features further enable positioning of sufficient amount of additional smaller microparticles or nanoparticles inside the topological features occupied by the microparticles wherein the smaller microparticles or nanoparticles assist mass spectrometric analysis of the analytes released from the microparticles. In an embodiment, the dimensions of topological features further enable deposition of liquid ionization matrix inside the topological features occupied by the microparticles in the amount sufficient for mass spectrometric analysis of the analytes released from the microparticles. In an embodiment, the density of the topological features on the solid support is between 1 and 1,000,000 per mm². In an embodiment, the device further comprises a separation seal or a separation gasket that restricts the microparticles to a specific area of the solid support. In an embodiment, the device further comprises a plurality of optic fibers wherein each topological feature is functionally connected to at least one optic fiber. In an embodiment, the optic fibers functionally connect the plurality of topological features to an optical detector. In an embodiment, the optic fibers enable measurement of optical properties of the analytes or optical properties of the microparticles positioned inside the topological features. In an embodiment, the device comprises a target plate for desorption-ionization mass spectrometry. In an embodiment, region of the solid support interrogated by the optic fibers coincides with interior of the topological features. In an embodiment, the microparticles positioned inside the topological features within the solid support comprise a microfluidic device. In an embodiment, the solid support is measuring approximately 25×75×1 mm or approximately 70×75×1 mm.

In an embodiment, a kit for analysis of microparticles by mass spectrometry comprises the device according to embodiments described above and ionization matrix wherein the ionization matrix is selected from a group comprising liquid MALDI matrices, microcrystals of solid MALDI matrices and nanoparticles. In an embodiment, the ionization matrix is selected according to nature of the analytes conjugated to the microparticles.

In an embodiment, a microarray system comprises an array of microspots and an array of microparticles wherein the two arrays are localized on the same solid support and individual elements of the arrays are spatially related. In an embodiment, the array of microspots contains analytes detectable by desorption—ionization mass spectrometry. In embodiment, at least some individual microspots or individual microparticles possess distinctive and measurable optical properties. In embodiment, the solid support is a microwell array plate.

In embodiment, a method of sample measurement comprises providing an array of analyte-containing microspots on a solid support wherein the array is fabricated from a group of microparticles and individual microspots are identified with individual precursor microparticles, providing a data acquisition protocol, and acquiring mass spectrometric data from the array of microspots according to the data acquisition protocol. In embodiment, the data is acquired using methods of mass spectrometry imaging. In embodiment, lateral resolution of the mass spectrometric data acquisition is between 1 micron and 1000 micron. In embodiment, the array of microspots contains between 1,000 and 10,000,000 analyte spots. In embodiment, the method of mass spectrometry is selected from a group comprising Matrix-Assisted Laser Desorption Ionization (MALDI), Desorption Electrospray Ionization (DESI), Laser Ablation Electrospray Ionization (LAESI), Desorption/Ionization on Silicon (DIOS), Nanostructured Laser Desorption Ionization (NALDI) and Secondary Ion Mass Spectrometry (SIMS). In embodiment, the method of mass spectrometry is selected from a group comprising TOF, TOF-TOF, Orbitrap, Quadrupole, Ion Trap, FT-MS, FT-ICR, Hybrid and Tandem mass spectrometry. In embodiment, the analytes are compounds selected from a group comprising polypeptides, peptidomimetics, proteins, nucleic acids, lipids, carbohydrates, small molecules, fragments of the above compounds and combinations of the above compounds. In embodiment, the microparticles are microbeads. In embodiment, parameters of the data acquisition protocol are selected from a group comprising: coordinates of an area on the solid support, coordinates of individual pixels on the solid support, distance between individual pixels, diameter of the ionization beam, intensity of the ionization beam, MS measurement mode, ion detection mode, spectral resolution, m/z detection range, number of averaged mass spectra per pixel and precursor ion for MS-MS measurement. In embodiment, specific numerical values of at least some data acquisition parameters are provided for individual microspots, individual groups of microspots or individual regions within the array. In embodiment, the numerical values of the data acquisition parameters are determined based on properties of the array, properties of the microparticles or method of fabrication of the microparticles. In embodiment, the numerical values of the data acquisition parameters are determined based on optical properties of the array or optical properties of the microparticles. In embodiment, the method further comprises the step of producing a mass spectrometric microarray dataset in a format suitable for image analysis. In embodiment, the method further comprises the step of analyzing the mass spectrometric data to detect analytes in individual microspots or analytes on individual microparticles. In embodiment, the analytes are detected quantitatively.

In embodiment, a method of analysis of biochemical reactions comprises the steps of providing a microarray dataset wherein the dataset is generated by mass spectrometric measurement of an array of analyte-containing microspots fabricated from a group of reacted microparticles, optionally applying methods of data processing to the microarray dataset, and analyzing the microarray dataset. In embodiment, analyzing the microarray dataset constitutes determining occurrence of a biochemical reaction or the absence thereof, extent of a biochemical reaction, direction of a biochemical reaction, time course of a biochemical reaction, type of a biochemical reaction or the number of distinct biochemical reactions. In embodiment, the biochemical reaction is affinity binding, small molecule binding, formation of a molecular complex, substrate modification by an enzyme or receptor-ligand binding. In embodiment, analyzing the microarray dataset constitutes interaction profiling, expression profiling, or functional identification. In embodiment, the microarray dataset is a microarray image. In embodiment, the microarray dataset additionally comprises time-dependent data. In embodiment, analyzing the microarray dataset constitutes quality control analysis. In embodiment, the method further comprises analyzing an optical dataset wherein the optical dataset is generated by optical measurement of individual microspots or individual microparticles. In embodiment, the method further comprises correlating optical and mass spectrometric data for individual microspots. In embodiment, the method further comprises identifying compounds on microparticles. In embodiment, the method further comprises providing a list of m/z values associated with individual analytes. In an embodiment, the list of m/z values is generated based on properties of the microparticles, method of the array fabrication and method of the mass spectrometry measurement.

The present disclosure is described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the present disclosure, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for.

In particular, the experimental procedures and methods utilized in the transfer of analytes from microbeads to the solid support are described in detail below. A brief description of experimental procedures involved in fabrication of analyte-conjugated microbeads, which were used to demonstrate the methods of analyte transfer is also given, however the latter examples are merely representative and should not be used to limit the scope of the present disclosure. A large variety of alternative bead designs exist, which may be used in the experimental workflow disclosed here. The selected examples are therefore used mostly to demonstrate the principles of the methods disclosed herein.

### EXAMPLES

Materials and Methods

Monodisperse Agarose Microbeads

The microbeads used in the experiments shown below are 6% cross-linked NHS-activated agarose beads (NHS HP SpinTrap, average particle size 34 micron) available from GE Healthcare Life Sciences (Piscataway, N.J.).

Protein Attachment to Microbeads

NeutrAvidin Protein (Invitrogen, Carlsbad Calif.) or anti-HSV monoclonal antibody (EMD Biosciences, Inc., San Diego Calif.) were covalently linked to the NHS-activated microbeads according to the manufacturer's protocol. To fabricate microbeads conjugated to both NeutrAvidin and anti-HSV monoclonal antibody, an equimolar mixture of the proteins was prepared at the concentration of 1 mg/mL and the protein binding to microbeads was performed according to the manufacturer's protocol.

Peptides Conjugated to Microbeads Using a Photolabile Linker

Photo-labile polypeptides were prepared by conjugation of an NHS-activated photo-labile biotin moiety to the peptide N-terminal amino group as described previously (Olejnik et al. Proc Natl Acad Sci USA 1995). The biotinylated photo-labile peptides were bound to NeutrAvidin coated beads as described previously (Olejnik, Sonar et al. Proc Natl Acad Sci USA 1995).

Fluorescent Peptide Conjugated to Microbeads

The HSV peptide (Sigma-Aldrich, St. Louis Mo.) was mixed with the Cy3-NHS fluorescent reagent (GE Healthcare Life Sciences, Piscataway N.J.) in a 1:1 molar ratio according to the manufacturer's protocol and the reaction was allowed to proceed overnight. The fluorescent peptide was bound to anti-HSV microbeads using standard protocols.

Cell-Free Expressed Protein Conjugated to Microbeads

Human p53 was expressed in a cell-free transcription/translation coupled rabbit reticulocyte lysate system TNT T7 (Promega, Madison, Wis.) by incubation of 5 μL of expression plasmid with 50 μL of cell-free reaction mixture for 2 hours at 30° C. The expression plasmid contained the full-length p53 cDNA sequence with an additional C-terminal 6×-His tag and HSV (QPELAPEDPED) tag. After 2 hours of incubation, the transcription/translation reaction was mixed with the suspension containing approximately 10,000 microbeads conjugated to anti-HSV antibody and incubated for 30 min. The beads are subsequently washed with a 10-fold volume of TBS-T buffer (twice), TBS buffer (twice) and deionized H₂O (twice) and stored in deionized H₂O.

Microwell Array Plates

The fiber optic glass microwell plates are manufactured by INCOM Inc (Charlton Mass.) from fiber optic bundles using the glass drawing technology. The microwell plates contain hundreds of thousands of miniature wells arranged in a hexagonal order and connected to a network of optical fibers designed primarily for the fluorescence and luminescence assay readout (FIG. 4). Each glass plate is custom made with respect to the plate overall dimensions, the diameter and depth of the microwells and the well-to-well distance. In the examples below, the microwell plates are Rectangular Fiberoptic Faceplates with Corner Chamfers and Side Bevels 75.0 mm×25.0 mm×1.0 mm thick (2.953″×0.984″×0.039″ thick). The material is Block Press BXI84-50 with Interstitial EMA. The fiber size is 50 micron. One side is etched to either 50 or 55 micron depth using selective removal of core glass by acid etching. In several examples, one side is etched to 35 micron depth. Each plate contains over 700,000 individual wells, 42 micron in diameter with 50 micron well-to-well spacing.

Conductive Microwell Array Plates with Hydrophobic Coating:

A thin layer of Gold was deposited on the surface of microwell array plates using the physical vapor deposition (PVD) process (Thin Films Inc, Hillsborough N.J.). The glass substrate was cleaned by water rinse and vapor dry, a 5 nm adhesion layer of Titanium was deposited directly on the substrate and a 5 nm layer of Gold was deposited on the adhesion layer of Titanium. Because the conductive layer was applied as a thin film, the solid support remains optically transparent.

Loading of Microbeads Onto a Microwell Array Plate

The process of depositing a library of microbeads into individual wells on the array plate is performed similarly to that described previously (Leamon et al. Electrophoresis 2003). Briefly, the open well side of a single 25×75×1 mm microwell plate is covered with the four-lane bead rubber loading gasket and the plate-gasket assembly placed into the size-matching PicoTiterPlate device, both available from 454 Life Sciences Corporation (Branford Conn.) and Roche Applied Science (Indianapolis Ind.). Microwells of the plate are optionally pre-filled with deionized H₂O prior to the bead loading. The bead suspension in aqueous medium is applied to the plate by manual or automated pipetting and beads are distributed throughput an area within the plate, which is defined by the loading gasket geometry, using repeated pipetting or by placing the entire assembly on a standard laboratory nutator. Beads initially settle into individual microwells by gravity and further placed near the bottom of wells by centrifugation of the entire PicoTiterPlate assembly at 2000 rpm for 15 min at room temperature. The plate is removed from the PicoTiterPlate device and its surface rinsed with deionized H₂O. Microbeads loaded into the microwells remain stable for several days before analysis when stored in a humidified container, preferably in the cold (4° C.) and dark environment.

Release of the Analyte from Microbeads by UV Irradiation:

The beads deposited on a microarray plate preferably remain hydrated prior to their exposure to the UV irradiation, for example by keeping the plate inside a humidified container or by covering the surface of the plate with a microscope glass coverslip. For the photolabile compounds used here and many other commercially available photocleavable reagents, the photorelease is achieved by a brief, 5 minute exposure to the near-UV light such as that provided by the Blak-Ray Lamp Model XX-15 (UVP, Upland Calif.). The power output of this source is 2.6 mW/cm² at 360 nm with the maximum output near 365 nm. The optimal distance between the UV source and the sample is between 2 and 10 cm. After the photorelease, the plate may be further incubated in a humidified environment or immediately coated with the MALDI matrix—containing solution.

Release of Analytes from Microbeads by Trypsin:

Mass spectrometry grade trypsin (Sigma-Aldrich, St. Louis Mo.) is diluted in deionized H₂O to the final concentration of 30 μg/mL. Approximately 5 mL of trypsin solution is loaded into a LC Sprint model reusable nebulizer (PARI Respiratory Equipment, Midlothian Va.) equipped with the TREK S compact compressor. The microarray plate with beads was placed into a closed container connected to the nebulizer. The fine mist trypsin solution is continuously produced by nebulizer for 2 minutes and allowed to settle on the plate. The plate is incubated within the same sealed container for 45 minutes at 37° C. After the incubation, the plate is coated with MALDI matrix as described below and the analyte is analyzed by MALDI TOF mass spectrometry.

Application of MALDI Matrix Solution to the Array Plate:

Mass-spectrometry grade crystal CHCA (Sigma-Aldrich, St. Louis Mo.) is dissolved in 60% acetonitrile, 0.1% trifluoroacetic acid (TFA) to the final concentration of 16 mg/mL. Approximately 5 mL of CHCA solution is loaded into a LC Sprint model reusable nebulizer (PARI Respiratory Equipment, Midlothian Va.) equipped with the TREK S compact compressor. The microarray chip is placed inside a closed container connected to the nebulizer. The application of CHCA matrix to the chip is performed in multiple cycles. Each cycle comprises the steps of matrix deposition, incubation and purging. During the step of matrix deposition, fine mist solution of CHCA is produced by nebulizer and allowed to settle on the chip. During the incubation step, the CHCA solution remains on the chip. During the purging step, the chip is allowed to air dry. The duration of each of the three steps is 20 seconds per step to the total of 1 minute per cycle. A total number of 10 cycles is sufficient to produce a layer of CHCA matrix suitable for the MALDI MS analysis. In addition to facilitating analysis by MALDI mass spectrometry, the above procedure is also used to release the analytes that are bound to the microbeads by acid-labile bonds, such as the antibody-antigen interaction, or by hydrophobic interactions.

Microwell Array Scan by Mass Spectrometry:

The measurements are performed on the ABI 4800 MALDI TOF/TOF mass spectrometer (AB Sciex, Foster City Calif.) equipped with the 4000 Series Explorer™ software. The image acquisition is performed using the 4000 Series Imaging software available in the public domain (www.maldi-msi.org). The typical image of a polypeptide microarray is collected in the MS reflector positive mode in the 650-3,500 Da mass range. The sampling bin size is 0.5 ns. The number of acquisition laser shots per spot is 100. The laser position remains fixed within a particular spot during the data acquisition. The rectangular area selected for the imaging experiment is determined by the (x1,y1-x2, y2) set of coordinates, which are entered either manually or interactively within the 4000 Series Imaging software. The raster size, which is the distance between adjacent spots on the microarray probed by the laser beam, is set to 40 micron in both x and y directions. The microarray scan comprises stepwise movement of the instrument sample plate with the mounted microarray plate by the raster distance with the data acquisition performed at each position. The data is collected and stored in the Analyze 7.5 format. The pattern of spots (pixels) in the microarray image obtained using the data collection protocol described above does not necessarily coincide with the pattern of individual analyte spots on the measured microarray, which are determined by the arrangement of microwells on the array plate. However, an alternative protocol of data acquisition can be implemented, in which the probing laser beam is initially positioned over the center of a first microwell to be measured and the data acquisition parameters are set to match the parameters of the microwell array plate. Specifically, the raster distance is selected to be equal to the distance between the centers of adjacent microwells with the pattern of spots to be measured matching the grid of microwells on the plate. The latter protocol can be easily implemented on most modern mass spectrometers, which are equipped with a high-resolution video camera capable of visualizing individual microwells and the software that allows the instrument user to create and implement custom scan patterns.

MS Image Data Analysis:

Array scans produced by MALDI TOF mass spectrometry imaging are analyzed using the program BioMap available in the public domain (www.maldi-msi.org). The array images showing distribution of a particular analyte on the microarray slide were produced by selecting the molecular weight of that analyte as the “mass channel” in the BioMap software. Normally, the position of the maximum of the analyte monoisotopic peak was selected as the appropriate mass channel. The intensity scale was manually adjusted in each case and the lower cut-off level for the spot display was selected to be approximately three times above the noise level. Thus, the positive spots in the microarray images, which are shown in white, are areas with the analyte signal at least three-fold above the noise level. The black background represents areas where the signal in the particular “mass channel” was below the threshold.

Microwell Array Scanning by Fluorescence:

Microwell array plates with the fluorescent analytes are scanned using a GenePix 4200A laser based microarray scanner (Molecular Devices, Sunnyvale, Calif.) at one or more excitation wavelengths at 488, 532, 594 and 635 nm depending on the nature of fluorophore. The pixel resolution is set to 10 micron and in some cases to 5 micron. The signal is acquired from the bottom of the microwell plate through the fiber optic channels. In order to measure eluted analytes the microwell plate is scanned in the “upside down” configuration with signal acquired from the surface containing openings into microwells. The focus offset is set according to the manufacturer's manual, typically between 0 and 50 micron.

Experimental Examples and Results

Some of the experiments performed using the methods described in this application and the resulting experimental data are shown below:

### Example 1

Sufficient analyte binding capacity of individual microbeads for mass spectrometric detection.

Recombinant polypeptide f-MKDYKDDDDKALYEICTEMEKEGKIFKIG (MW 3483 Da) was produced using PURExpress® In Vitro protein synthesis kit (New England BioLabs, Beverly Mass.) according to the manufacturer's instructions and captured on the anti-FLAG agarose beads. According to the manufacturer's manual, 5 μL of PCR DNA product was added to 50 μL of PURExpress kit. After 2 hours of translation reaction at 37° C., a 10-fold excess of buffer solution containing 10% Triton X-100 and 0.5% PBS (pH 8.0) was added to the mixture. The resulting solution was incubated with 1 μL of EZView anti-FLAG agarose beads (Sigma-Aldrich, St. Louis Mo.) for 15 minutes to allow for the polypeptide binding to beads. The EZView anti-FLAG agarose beads are polydisperse with diameter of individual beads in the 40-130 micron diameter. The beads were subsequently washed with deionized H₂O, randomly deposited on the surface of a microwell array plate (not inside the microwells) and sprayed with the MALDI matrix solution.

In reference to FIG. 24B, the MALDI TOF MS image of a small area within the plate shows spots on the array with the 3483 Da signal above the background. The random pattern of spots reflects the random arrangement of beads on the surface. Furthermore, the different size of spots is in agreement with the different size of beads, to which the 3483 Da analyte was attached. In this example, the analyte elution was performed by low-pH MALDI matrix, which disrupts the peptide-antibody interaction.

### Example 2

Selective elution and detection of a peptide analyte conjugated to microbeads via a photolabile linker.

Peptide YTDIEMNRLGK (VSV-G peptide, MW 1339.5 Da, AnaSpec, Fremont Calif.) was conjugated to 34 micron cross-linked avidin-coated agarose microbeads, using a photolabile biotin linker covalently attached to the peptide N-terminus. In the absence of UV irradiation, the biotinylated peptide remains conjugated to the beads due to the strong avidin-biotin interaction.

Several thousand peptide-conjugated microbeads suspended in deionized water were applied to the surface of a 75×25×1 mm rectangular fiber optic microwell plate. Beads were deposited within a 45×2.5 mm rectangular area by using a rubber gasket during the bead deposition.

A section of the plate was irradiated for 5 minutes by the near-UV light (365 nm maximum output) while the remaining part of the plate was protected from the UV light. The UV-irradiated and non-irradiated areas of the plate comprised areas with loaded microbeads, as well as areas with no beads. Following the UV-irradiation, the MALDI matrix was uniformly applied to the entire plate and a subsection of the plate was imaged by MALDI TOF mass spectrometry. The imaged area comprised both irradiated and non- irradiated sections of the plate.

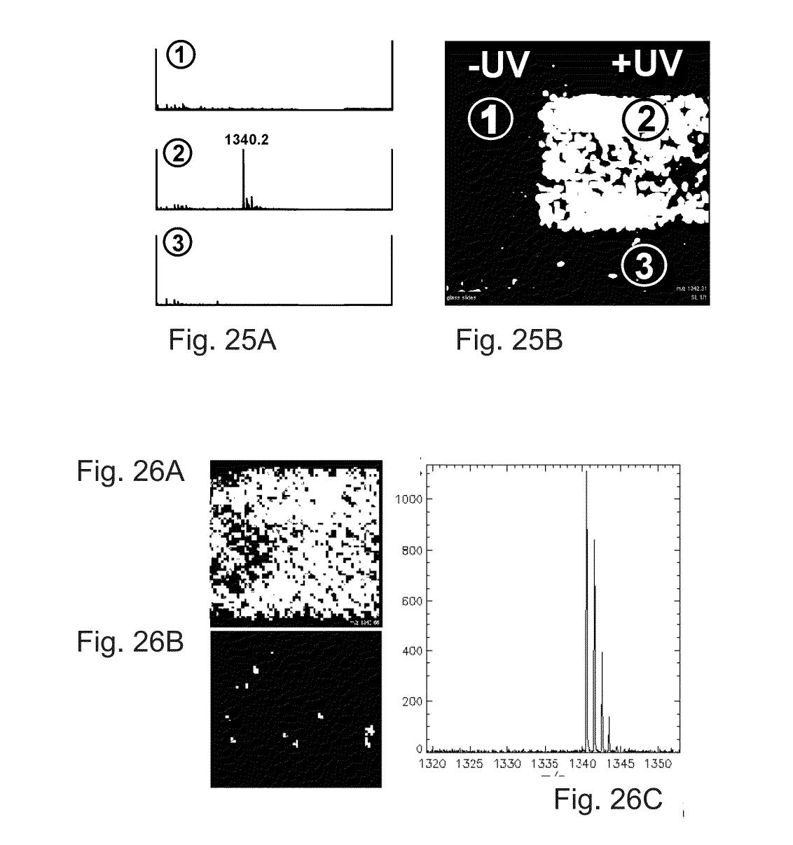

The experimental results are shown in FIG. 25A and FIG. 25B. FIG. 25A shows representative single spot mass spectra obtained from: (1) an area of the array with loaded beads where no UV irradiation was applied; (2) an area of the array with loaded beads, which was exposed to UV irradiation for 5 minutes; (3) an area of the array devoid of beads, which was exposed to UV irradiation for 5 minutes. FIG. 25B shows MALDI TOF MS image of the section of an array labeled with locations of the three areas described above. The data demonstrates that UV-irradiation of the microbeads deposited on the microwell array plate allows photorelease of the intact analyte in the amount sufficient for the subsequent detection by mass spectrometry. Specifically, only within the irradiated area of the plate, which also contained beads (area 2), a signal was observed at the expected molecular weight of the original VSV-G peptide. The recorded signal was strong (up to 1000:1 signal-to-noise ratio). Furthermore, overall shape of the area where the signal was observed matched the area where the beads were deposited and irradiated. In contrast the mass spectra recorded from beads that were not irradiated (area 1) as well as the area containing no beads (area 3) had very weak signal essentially within the noise level.

### Example 3

Dense packing of microbeads on the microwell array plate at approximately 50% occupancy.

Two populations of beads were prepared. The “positive” population comprised agarose microbeads conjugated to the VSV-G peptide via the photolabile biotin linker. The “control” population comprised 98% of blank beads (no VSV-G peptide) and 2% of photolabile VSV-G peptide conjugated beads. The bead populations were deposited into two separate areas on the microwell array plate. In both cases the number of beads was calculated to provide an approximately 50% bead per well occupancy (1 bead per 2 microwells). Using microwell plates with well-to-well distance of 50 micron, an average density of approximately 200 spots per mm² was achieved. The plate was UV-irradiated and coated with MALDI matrix.

FIG. 26A shows MALDI TOF MS image of an array fabricated from beads carrying the 1340 Da VSV-G peptide. FIG. 26B shows MALDI TOF MS image of an array fabricated from a mixture of 98% blank beads and 2% VSV-G peptide beads. A significantly greater spot density is observed in FIG. 26A, as expected. FIG. 26C shows strong VSV-G peptide signal obtained in the MS reflector mode, which is recorded from a single spot. Note that the UV irradiation enables recovery and detection of the intact VSV-G peptide.

### Example 4

Converting a large bead library into an array of microspots, which is measured by mass spectrometry.

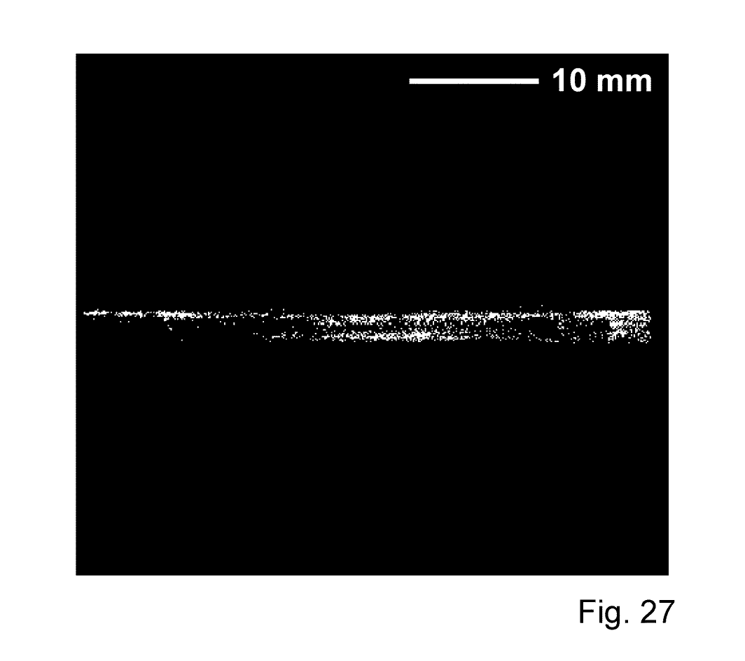

An approximately 10,000 microbeads suspended in deionized H₂O were loaded onto the microwell array plate within a 45×2.5 mm rectangular area. Each microbead was conjugated to VSV-G peptide (MW 1340 Da) via photolabile biotin linker. The polypepitde was released from the beads by UV irradiation and mixed with the MALDI matrix. An area of the chip encompassing the bead deposition area was measured by mass spectrometry in the imaging mode. FIG. 27 shows an area of the chip where the 1340 Da peak was detected. Some individual spots can be seen along with the general distribution of beads on the plate. In particular, the rectangular shape of area where the analyte was detected matches the shape of the rubber gasket used to restrict the bead spread. The non-uniform distribution of beads detected by mass spectrometry with areas of noticeably higher bead density near the top and bottom edges matches the pattern observed in experiments where the presence of beads is detected by fluorescent or colorimetric array scanning With microwells separated by 50 micron, the total number of microwells in the 45×2.5 mm area is calculated to be approximately 50,000. Thus, a 10,000 member bead library represents an approximately 20% occupancy which is in agreement with the observed data.

### Example 5

Uniform spots of analytes fabricated by controlled photorelease of the analyte from the microbeads and application of the MALDI matrix.

The analyte is a polypeptide RPPGFSPFR (Bradykinin, MW 1060 Da, AnaSpec, Fremont Calif.) conjugated to 34 micron monodisperse avidin-coated agarose microbeads using a photolabile biotin linker covalently bound to the peptide N-terminus. A suspension of microbeads in deionized H₂O was deposited inside microwells on the microwell array plate using the standard bead loading procedure.

The microwell plate with loaded microbeads was exposed to long wavelength UV light for 5 min. Following the photoelution, MALDI matrix solution was applied to the microwell plate using the spray deposition technique. The fabricated array of microspots was scanned by MALDI TOF mass spectrometry in the imaging mode.

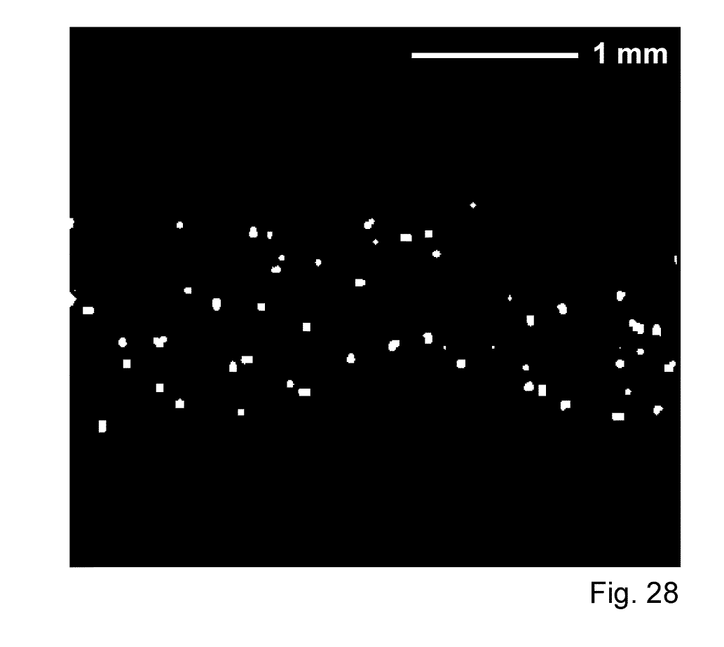

The MS image of the scanned region is shown in FIG. 28. Areas with the strong 1060 m/z signal appear as compact, uniform and well-defined spots. The data indicates that the analyte is efficiently released from the microbeads by UV irradiation, retained in the vicinity of the microbeads and remains accessible to the laser ionization beam of the mass spectrometer.

### Example 6

Independent detection of the analyte by fluorescence and mass spectrometry imaging of the same microarray.

The analyte is the HSV peptide (KQPELAPEDPED) covalently linked to the fluorescent marker Cy3 at the peptide N-terminus. The molecular weight of fluorescent HSV peptide is 2047 Da. The peptide is bound to the 34 micron agarose beads coated with an anti-HSV antibody. The beads were deposited on the microarray plate and coated with MALDI matrix solution as described previously. In this example, the elution of analyte from beads results from the low pH of the MALDI matrix, which disrupts the antibody—peptide interaction.

The fluorescence scan of the peptide spot array was performed using GenePix 4200 microarray scanner. Independently, mass spectrometric MS scan of the same peptide array was performed using MALDI TOF mass spectrometer.

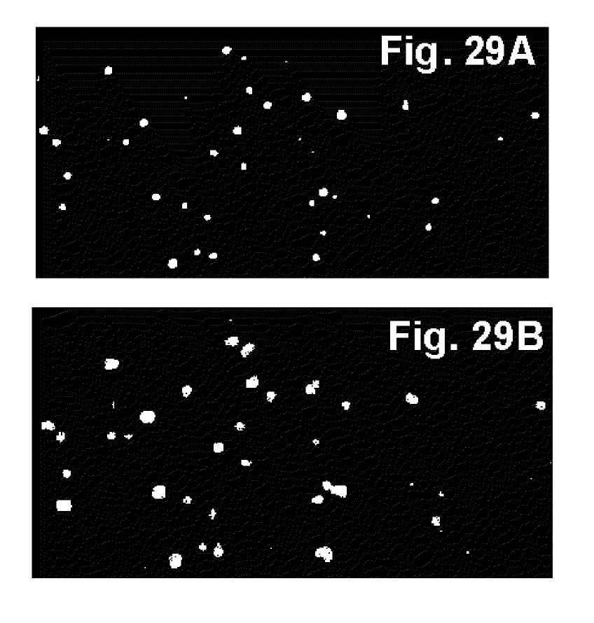

FIG. 29A and FIG. 29B show comparison of fluorescent imaging of Cy3 label performed in the 532 nm channel (FIG. 29A) and MS imaging of the intact peptide performed in the 2047 Da channel (FIG. 29B). The two images show very similar pattern of spots indicating that the same analyte is detected by two independent methods. Importantly, the spatial resolution and sensitivity of the mass spectrometric detection are similar to those obtained by fluorescence detection in this example.

### Example 7

Co-registration of mass spectrometric and optical images of a polypeptide array and a fluorescent microbead array.

The analyte is Bradykinin polypepitde (MW 1060 Da) conjugated to NeutrAvidin -coated 34 micron agarose microbeads via the photolabile biotin group linker. The NeutrAvidin protein is additionally labeled with the fluorescent Cy5 marker using a photostable linker. The bead library was loaded into microwell array plate, exposed to the near-UV light for 5 min and subsequently coated with the MALDI matrix.

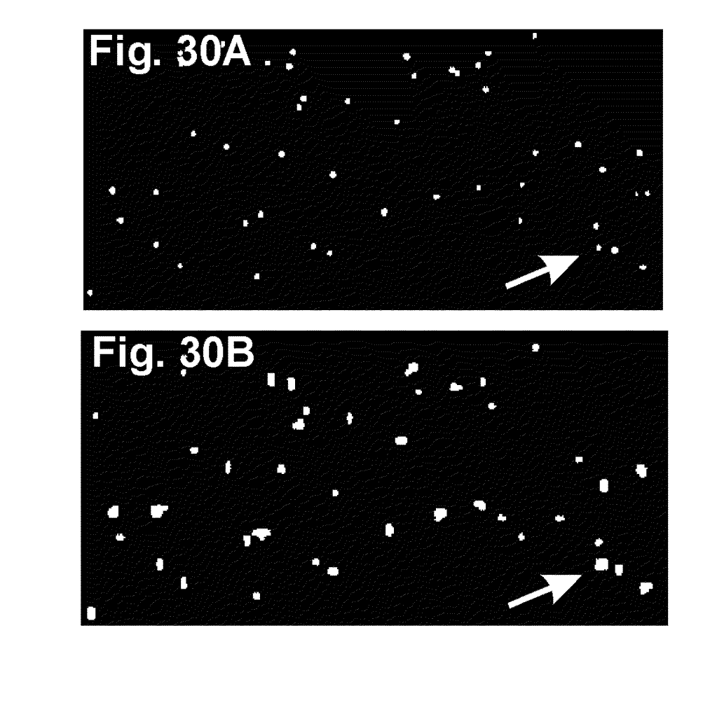

The plate is imaged by fluorescence and MALDI MSI. FIG. 30A and FIG. 30B show both the fluorescent and MALDI TOF MS images, respectively, of the same microarray area. An arrow points to the same spot on both images. A comparison of the two images reveals nearly perfect correlation between the fluorescent labels attached to the microbeads and the peptide analyte deposited on the surface of the microarray plate.

The experimental data demonstrates that 34 micron agarose beads have sufficient capacity to bind both the peptide analyte for the mass spectrometric detection and the fluorescent label for fluorescence detection. The data shows feasibility of using a set of unique fluorescent markers to provide individual microbeads with a unique fluorescence signature that also allows independent detection by mass spectrometry in a microarray format.

Furthermore, this example shows two separate but spatially related arrays fabricated from the precursor bead library that are located on the same microwell plate. The first is a planar microarray comprising multiple spots on the surface formed by the eluted polypeptide analyte. The second is the bead microarray comprising multiple beads located inside microwells with the fluorescent analytes conjugated to beads. The two arrays share the same geometry, yet they are distinct and measured by two independent methods, namely mass spectrometry and fluorescence.

### Example 8

Co-elution of fluorescent and polypeptide analytes from individual microbeads.

The microbeads are monodisperse 34 micron agarose microbeads conjugated to an equimolar mixture of NeutrAvidin and anti-HSV monoclonal antibody (EMD Biosciences, Inc., San Diego, Calif.). Polypeptide WQPPRARI (MW 1023 Da) is conjugated to the microbeads via the photolabile biotin linker attached to the peptide N-terminus and the biotin—NeutrAvidin bridge. The polypeptide serves solely as the molecular weight marker. Cell-free expressed purified full-length human p53 protein with a C-terminal HSV tag is bound to the same microbeads via the HSV tag—anti-HSV antibody linkage. Alexa-594 fluorescently labeled anti-p53 antibody is bound to the bead-conjugated p53 protein.

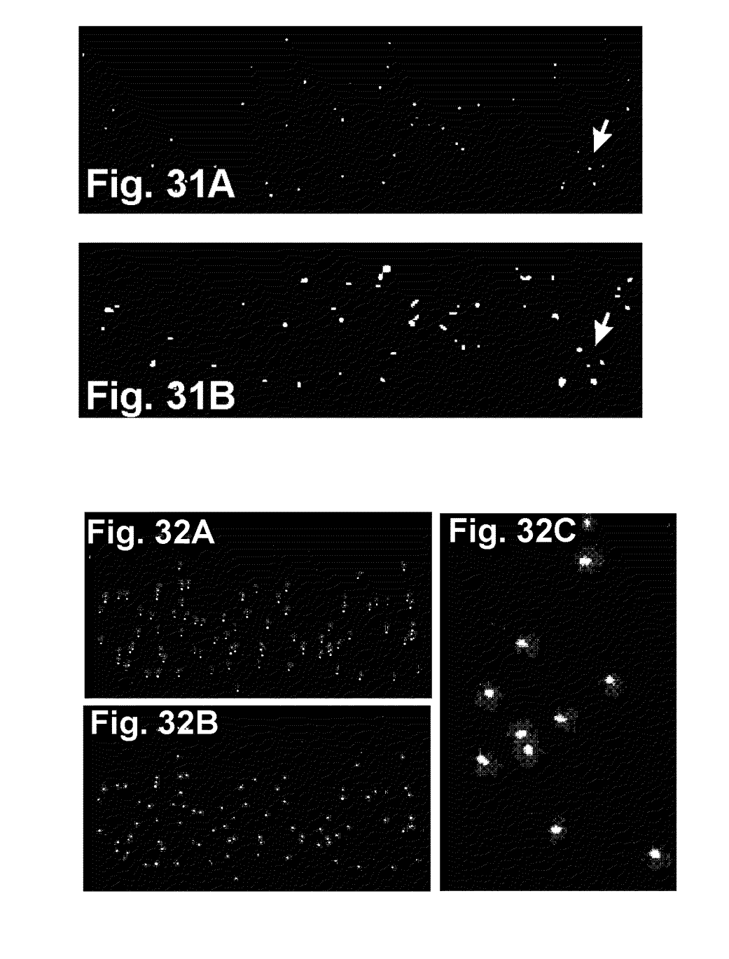

The bead library is deposited on the microwell array plate, UV-irradiated and coated with the MALDI matrix solution. This procedure results in elution of both the 1023 Da polypeptide and the fluorescent antibody. The fabricated array of microspots is independently imaged by fluorescence scanning in the 594 nm channel and MALDI TOF mass spectrometry in the 1023 m/z mass channel. FIG. 31A and FIG. 31B show fluorescence detection of the Alexa-594 labeled anti-p53 antibody and detection by MALDI TOF MS of the 1023 Da peptide, respectively. An arrow points to the same spot on the array. The two images are very similar indicating co-localization of the fluorescent and peptide analytes on the microwell array plate after the elution. In this example, the peptide analyte is released from beads by UV-irradiation, while the fluorescent label is released by application of the low-pH MALDI matrix solution, which disrupts the protein-antibody interaction.

### Example 9

Optical readout of eluted and bead-bound fluorescent analytes

The analyte is a fluorescent marker Cy5. The monodisperse 34 micron agarose microbeads are coated with NeutrAvidin, which is labeled with Cy5 using amino-reactive Cy5 NHS ester. The microbeads were deposited on unmodified (glass surface) microwell array plate, spray-coated with a solution containing 60% acetonitrile and 0.1% TFA and further exposed to the concentrated vapor containing 60% acetonitrile and 0.1% TFA for 1 hour at 37° C. The prolonged exposure to organic solvent and TFA results in partial elution of Cy5-labeled NeutrAvidin from the microbeads, presumably due to dissociation of the individual protein subunits. The eluted analytes were allowed to migrate on the hydrophilic surface of unmodified glass microplates. After 1 hour of incubation, the plate is coated with MALDI matrix and imaged by fluorescence scanning at 635 nm from the bottom (fiber optic channels) and top (surface) of the microwell plate. In a separate control experiment beads with the same analyte were deposited on a microarray plate and coated with MALDI matrix but were not exposed to the acetonitrile/TFA vapor.

As shown in FIGS. 8A-8C, the optical image recorded from the bottom reflects the analyte inside the individual microwells, most likely still conjugated to the beads. The top image recorded from the surface of microwell plate through the layer of crystallized MALDI matrix reflects the analyte eluted from the beads and deposited on the plate surface.

FIG. 32A, FIG. 32B, and FIG. 32C show two fluorescence images recorded in 635 nm channel of an analyte-bead microarray after extended exposure to the TFA/acetonitrile medium, which results in excessive migration of the Cy5 fluorescent analyte. The images were recorded from the same area from the top (surface) and bottom (wells) of the same microarray and are shown as offset images in FIG. 32A and directly superimposed images in FIG. 32B with the zoom-in showing a section of the superimposed images in detail in FIG. 32C. The fluorescent image recorded from the bottom of the plate reveals very strong signal, localized to individual wells. The signal intensity is near the detector saturation limit (FIGS. 32A and B). The image recorded from the microwell plate surface reveals lower intensity signal with 100% spot correlation indicating that sample extraction occurs from every microbead. Larger area of individual spots seen in the surface recorded image reflects excessive delocalization of the analyte due to the prolonged exposure to organic solvent. In fact, the analyte spots cover the area of several microwells with some adjacent spots merging. This allows close examination of the distribution of analyte on the surface following its extraction from microbeads. Superposition of the top and bottom images shows that beads are always located in the center of the spots formed by the analytes eluted from that particular bead (FIG. 32C). The distribution of analyte after its elution follows the radial pattern and is highly reproducible for all beads. The experimental data suggests that a relatively simple mathematical algorithm can be applied to the data, if needed, to correct for excessive diffusion and reconstruct the array images with higher resolution, up to a single well. Another conclusion is that separate optical imaging of the microwells via fiber optic channels and of microwell plate surface can be used to discriminate between the bead- bound and eluted analytes. Another important conclusion is that the fluorescence image can be recorded from the plate surface, which is covered with a layer of MALDI matrix. Therefore, the ability to perform fluorescence detection is not affected by the application of MALDI matrix to the plate.

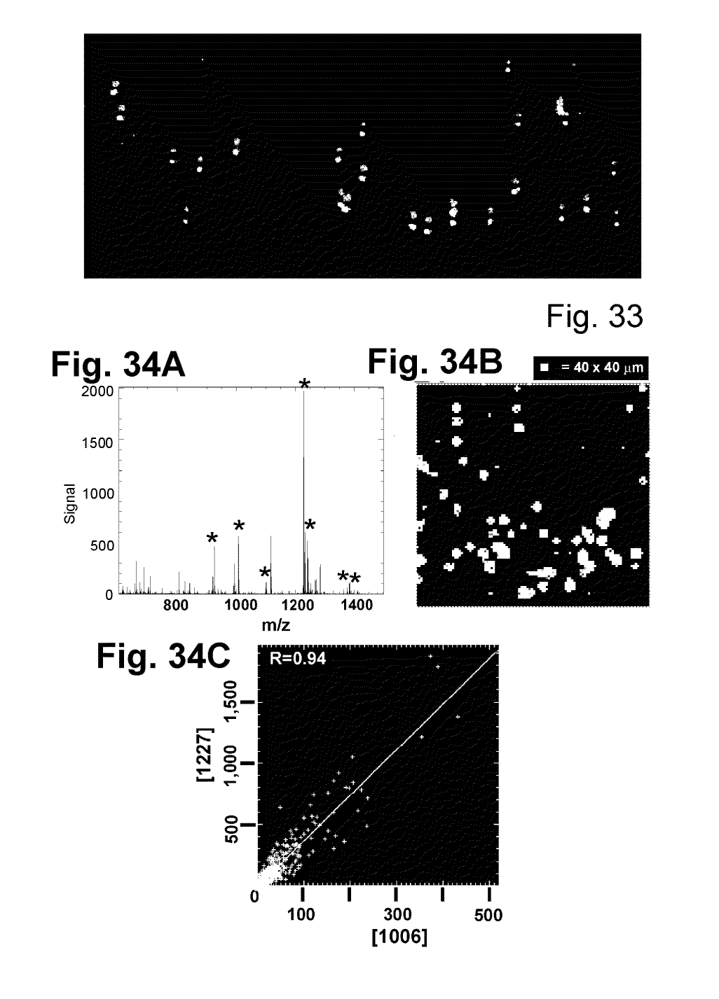

FIG. 33 shows the result of a control experiment performed as described above, except that the beads were not exposed to the acetonitrile/TFA vapors and were only coated with the MALDI matrix solution. Two images recorded from the surface and microwells were offset and combined to form a single image. The image comparison shows that almost no elution of Cy5 analyte occurs from beads exposed only to the MALDI matrix solution and without exposure to organic vapors.

### Example 10

Enzymatic reaction performed on bead-conjugated analytes arrayed on a microwell array plate.

The analyte is Red Fluorescent Protein (RFP) with a C-terminal HSV affinity tag conjugated to monodisperse 34 micron agarose microbeads coated with an anti-HSV antibody. The beads deposited inside individual microwells on a microarray array plate were exposed to a dilute aqueous solution of trypsin applied in a form of a fine mist and incubated in a humidified chamber of 45 min. Following the exposure to trypsin the microwell plate was coated with MALDI matrix and MS imaging of the plate was performed.

FIG. 34A shows an exemplary single-spot MALDI TOF mass spectrum of RFP after on-the-slide exposure to trypsin. Peaks in the mass spectrum, which are assigned to the specific RFP fragments, are labeled with an asterisk. The MALDI TOF MS image of the microwell array plate shows distribution of the 1227 Da peak corresponding to the molecular weight of one of the segments of digested RFP, as shown in FIG. 34B. The 40×40 micron size pixel is shown for comparison. Also shown is the scatter plot demonstrating correlation between the intensity of 1006 and 1227 Da peaks, both of which are specific for the RFP digest, for every pixel of the array, as shown in FIG. 34C. Overall, mass spectra recorded from individual spots of the microarray exhibit multiple peaks consistent with the protein digestion of RFP by trypsin. Furthermore, the spatial resolution of the array is not significantly decreased due to the application of trypsin as most individual spots arising from individual beads are still resolved and their size is comparable to the size of microbeads. The good spatial resolution is likely due to a combination of the hydrophobic microwell surface and the presence of hydrophilic agarose microbeads that force individual droplets of the aqueous solution containing trypsin generated by the nebulizer to coalesce around the openings into the microwells. An important outcome of this experiment is that the ratio of intensity ratio of individual peaks in the mass spectra, which reflect digested protein fragments, remains nearly constant. This effect demonstrates reproducibility of digestion conditions throughout the microwell plate. The observed close correlation of peak intensity for fragments derived from the same protein may be used to confirm the assignment of multiple peaks to the original protein and therefore, to a specific bead.

This example shows the ability to remove the analyte from bead by applying a digestive enzyme. The enzyme can be selected to selectively fragment only the linker between analyte and bead. However, the enzyme can also be applied to digest the analyte for the purpose of its subsequent analysis by analytical methods, for example using protein mass fingerprinting method.

In addition to digestive enzymes, other enzymes or bioactive reagents can be applied to beads immobilized on the microarray plate to perform a variety of reactions on the analyte conjugated to beads. The enzymes can be later removed by rinsing the slide or remain on the slide. Multiple enzymatic reactions can be performed on the same slide, either concurrently or consecutively, providing an alternative to performing the reaction using suspensions of beads in solution.

### Example 11

Co-localization and quantitative co-elution of multiple analytes from individual microbeads.

Peptides PPGFSPFR (905 Da), WQPPRARI (1023 Da), RPPGFSfFR (f denotes D-Phe, 1110 Da) and APRLRFYSL (1122 Da) were from Anaspec (Fremont Calif.). The peptides were chosen solely on the basis of their molecular weight and used as MW markers. Each of the peptides was conjugated to the NHS-activated photo-labile biotin. The biotinylated 905, 1110 and 1122 Da peptides and separately 1023, 1110 and 1122 Da peptides were mixed in approximately 1:1:1 molar ratio in solution before binding to monodisperse 34 micron NeutrAvidin-coated microbeads. Two of the polypeptides were identical in both mixtures, while the third was different. The two populations of microbeads were mixed and loaded on the microwell array plate, peptides were eluted by UV-irradiation and MALDI matrix was applied to the microwell plate. The fabricated array of microspots was imaged using MALDI TOF mass spectrometry.

FIGS. 35A-35D show exemplary single spot mass spectra measured from an array of microspots fabricated from a bead library with two populations of microbeads with three distinct analytes attached to each bead, as shown in FIG. 35A and FIG. 35B. Also shown are intensity scatter plots demonstrating co-detection of the 1110 and 1122 Da peptide analytes, which are present on all beads, and 905 and 1023 Da peptide analytes, which are mutually exclusive, as shown in FIG. 35C and FIG. 35D, respectively). Note that each of the mass spectra recorded from a single microarray spot shows three strong peaks at the expected molecular weight with two peaks appearing at 1110 and 1122 Da in every positive spot on the array, with the third peak appearing either at 905 or 1023 Da. To confirm that the peptide analytes are indeed co-localized on the slide, intensity scatter plots for each polypeptide were constructed for every pixel of the array. The scatter plots show that the 1110 and 1122 Da peaks indeed appear together in every spot where the signal was detected (both intensities have a positive value) (FIG. 35C), while the 905 and 1023 Da peaks are mutually exclusive, i.e. the positive intensity for one of the peaks is accompanied by zero intensity for the other peak, so the data points are mostly observed on the X or Y axis (FIG. 35D). Several data points in the scatter plot for 905 and 1023 Da peaks, which display non-zero intensity for both peaks, most likely reflect the spot overlap on the microarray, i.e. two beads in close proximity.

Also, close correlation is observed between the intensity of the 1110 and 1122 Da peaks for every spot on the microarray. While the absolute intensity of each peak varies significantly between the spots, the ratio remains remarkably close—in fact the linear regression reveals the correlation coefficient R square of 0.95. In contrast, the correlation coefficient for the 905 and 1023 Da peaks is 0.00 (FIG. 35C and FIG. 35D). This data indicates that the distinct analytes are eluted and localized on the solid support in the same molar ratio, in which they were present on beads. Therefore quantitative analysis is possible for example by including an internal standard of a known concentration to the mixture of analytes prior to their binding to beads.

### Example 12

Co-elution of analytes from complex analyte-microbead constructs.

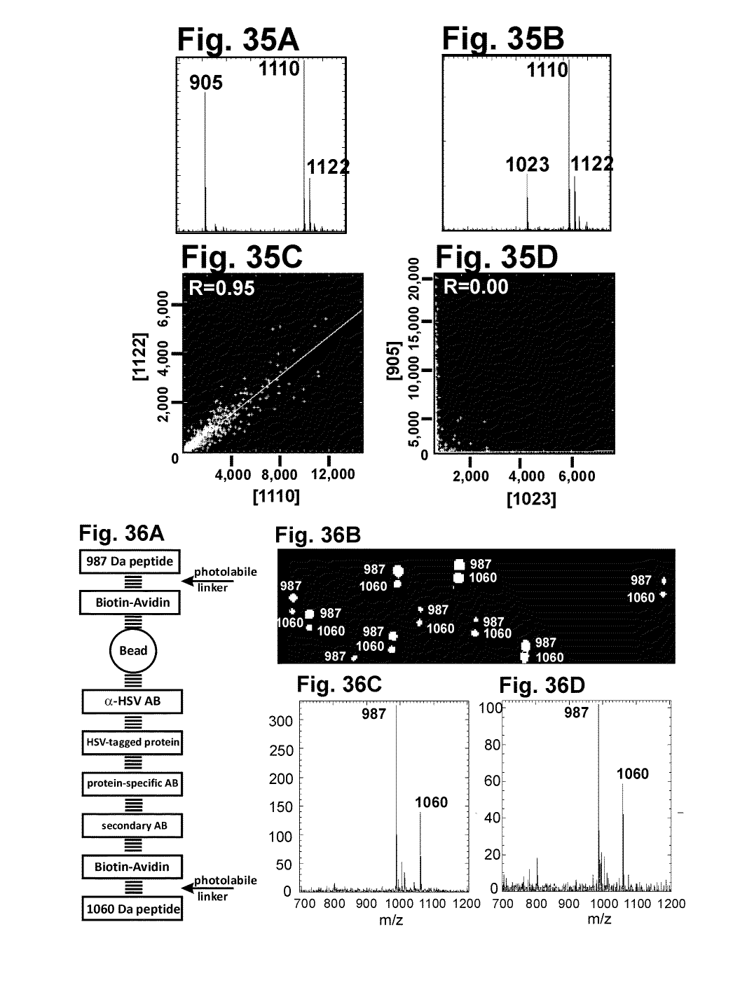

Monodisperse 34 micron agarose microbeads are conjugated to two distinct polypeptide analytes, as shown schematically in FIG. 36A. One of the peptide analytes (MIGGAGGRIR, MW 987 Da) is conjugated to the bead via the photolabile biotin—Neutravidin linkage, while the other peptide analyte (Bradykinin, MW 1060 Da) is conjugated via an HSV antibody—HSV tagged protein—protein specific antibody—biotinylated secondary antibody—NeutrAvidin - photolabile biotin construct. The microbeads were loaded on the microwell array plate, peptide analytes eluted by UV-irradiation and MALDI matrix was applied to the plate. The imaging was performed by MALDI TOF mass spectrometry. FIG. 36B shows MALDI TOF MS image of a resulting array of microspots with labels indicating the molecular weight of analyte in each spot. Note that the array images recorded in the 987 and 1060 m/z mass channels are intentionally offset to show spot correlation. The array images demonstrate that the shape, size and positions of spots containing the 987 and 1060 Da analytes, are very similar. In fact, almost perfect match was observed for the majority of spots containing these analytes. FIG. 36C and FIG. 36D show exemplary single spot mass spectra recorded from the microarray. The ratio of peak intensity for the 987 and 1060 Da peptides in the mass spectra recorded from different spots on the array is similar indicating that the ratio of two peptides on a bead is preserved in the microarray spots after the elution. This result is surprising considering that the 987 Da and 1060 Da peptides are in a different environment on the beads. While the 987 Da peptide is located near the bead surface, the 1060 Da peptide is conjugated via a complex protein-antibody construct and located further away from the bead surface.

This experimental data suggests that quantitative measurements of analytes, which are bound to microbeads, for example by antibody-protein interactions, can be performed by mass spectrometric measurement of a bead array if each bead is provided with an internal standard of known concentration. In this example, the 987 Da peptide analyte attached directly to the bead serves as an internal standard, while the 1060 Da peptide can be used to estimate the amount of protein bound to the antibody conjugated to the same bead.

### Example 13

Controlling elution and detection of analytes from microbeads by providing microarray plates of specific well depth.

Analyte-conjugated microbeads can be placed at a specific distance from the surface of the microwell plate, as shown schematically in FIG. 37, FIG. 37B and FIG. 37C. Specifically, the microbeads may be completely submerged in microwells, as shwon in FIG. 37A, placed near the surface of the microwell plate as shown in FIG. 37B or only partially submerged in microwells as shown in FIG. 37C. The distance between the beads and the surface of microwell plate controls accessibility of beads to elution reagents and accessibility of eluted analytes to the ionization beam of the mass spectrometer.

Monodisperse 34 micron agarose microbeads conjugated to Bradykinin polypeptide analyte (MW 1060 Da) via a photolabile linker were loaded on two microwell array plates featuring microwells 35 and 55 micron deep. Microbeads were loaded at the same density on the two plates. The plates with loaded beads were UV irradiated and coated with MALDI matrix solution using identical conditions.

FIG. 38A and FIG. 38B show MALDI TOF MS images recorded in the 1060 m/z mass channel for the microwell plate with the bead diameter/well depth ratio of 34/35 micron and the microwell plate with the bead diameter/well depth ratio of 34/55 micron, respectively. A greater number of spots with the signal above the background are seen for the 35 micron microwell plate compared to the 55 micron microwell plate. The signal intensity in each spot also appears to be higher on the 35 micron plate. The data suggests that at least under some experimental conditions, placing microbeads close to the surface leads to stronger signal from the eluted analyte.

### Example 14

Controlling on-bead enzymatic reactions by providing microarray plates of specific well depth.

Monodisperse 34 micron agarose microbeads coated with anti-HSV antibody and conjugated to HSV-tagged Red Fluorescent Protein (RFP) were loaded at the same density into two microwell array plates featuring 35 and 55 micron deep wells, respectively. In the former case, the beads are near the surface, while in the latter case, the beads are submerged in microwells. Both plates with beads were exposed to the solution of trypsin according to the procedure described previously and subsequently coated with MALDI matrix solution under identical conditions. The fabricated arrays of microspots were scanned by mass spectrometry imaging.

FIGS. 39A-39F show the MALDI TOF MS image comparison for two mass channels corresponding to specific fragments of the digested RFP: 1228 Da (Left Panel) and 1006 Da (Right Panel). For each mass channel the data was recorded from beads inside 35 micron wells (FIG. 39A, 39D), beads inside 55 micron wells (FIG. 39B, 39E) and blank beads without RFP inside 35 micron wells (FIG. 39C, 39F). The signal intensity observed for both proteolytic fragments arising from the digestion of RFP with trypsin is significantly higher for beads loaded into the 35 micron wells. In particular, many more spots with the 1227 and 1006 Da peaks above the background are detected on the 35 micron microwell plate compared to the 55 micron microwell plate. As a control, the 1227 and 1006 Da peaks are absent when blank beads RFP have been exposed to trypsin. The data suggests that, at least under disclosed experimental conditions, placing beads close to the surface provides greater accessibility of bead-conjugated protein to an enzyme.

### Example 15

Fabrication of an array of microspots containing both fluorescent and polypeptide analytes from microbeads smaller than 34 micron.

It is known that microbeads made of crosslinked agarose can undergo fragmentation after repeated mechanical agitation, for example vortexing, which results in formation of smaller fragments. Nevertheless, these fragments remain functional and retain the ability to bind the analytes. During the bead loading on a microarray plate, both regular size beads and the smaller fragments are deposited into the microwells.

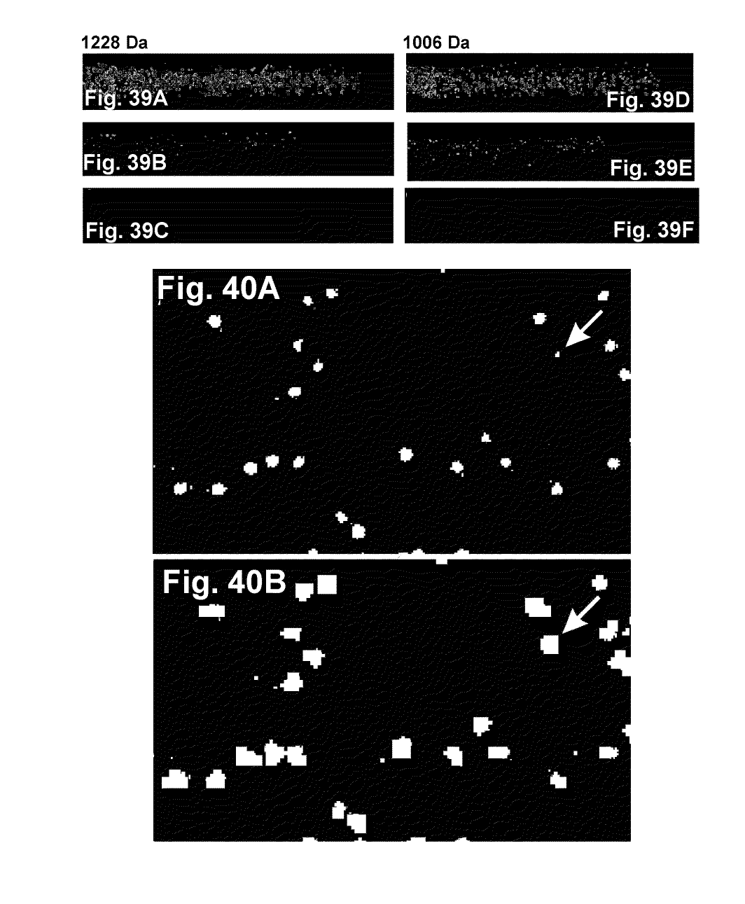

FIG. 40A and FIG. 40B respectively show the fluorescence and MALDI TOF MS images of a microwell array plate loaded with beads, which carry both a Cy5 fluorescent marker and 1060 Da Bradykinin peptide analyte attached via a photolabile linker, similarly to Example 7. The peptide analyte is eluted by UV-irradiation and application of the MALDI matrix, while the fluorescent marker remains conjugated to the bead. The resolution of the fluorescence scan was 5 micron, which is sufficient to detect smaller fragments. An arrow indicates the location of a bead fragment, which is significantly smaller than the regular 34 micron beads. This fragment was first detected by fluorescence (top image) and the comparison with the MALDI TOF MS image reveals a strong 1060 Da Bradykinin signal in that area indicating that sufficient amount of peptide was eluted from the bead fragment and detected by mass spectrometry. Elsewhere on the microarray plate, nearly perfect agreement was observed between the fluorescence and mass spectrometric images indicating that the observed effect is real. It is estimated that the spot marked by an arrow on the fluorescence image (FIG. 40A) is less than 10 micron in diameter. The use of smaller beads allows further increase of the density of beads on the plate, which can be beneficial for certain applications. For example, the use of microwells with 10 micron well-to-well separation increases the spot density to 10,000 per mm².

### Example 16

Fabrication of an array of microspots with individual spots similar to dimensions of a single microwell.

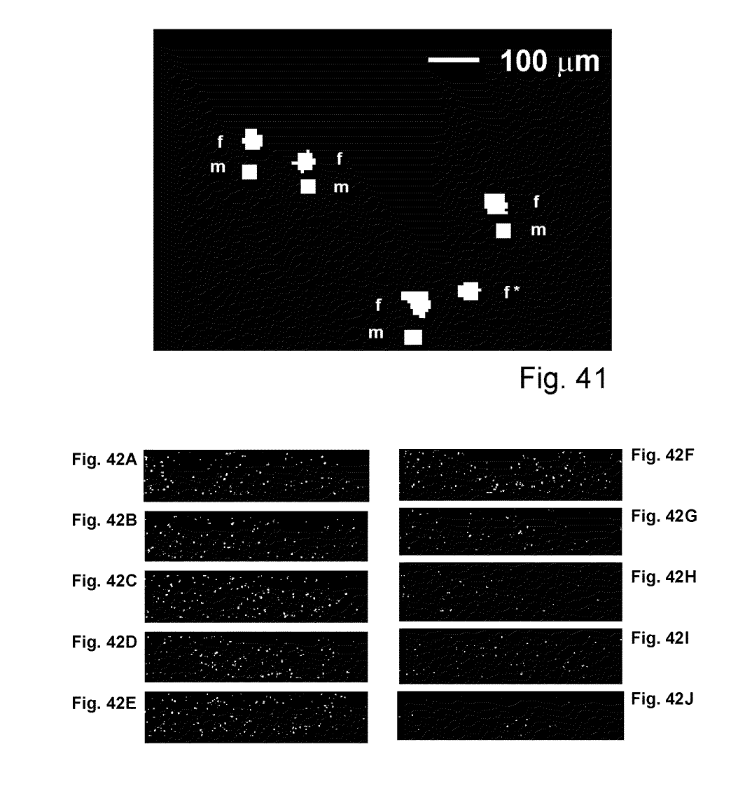

Monodisperse 34 micron agarose microbeads with a fluorescent Cy5 label and 1060 Da Bradykinin polypeptide analyte conjugated via a photolabile linker were loaded onto the microwell array plate by centrifugation. Suspension of crystalline CHCA matrix with individual crystals approximately 3 micron in diameter (Mass Spec Focus Chip Solvent Kit, Qiagen) in deionized H₂O was then applied to the plate by pipetting and deposited into wells on top of the microbeads by centrifugation. The excess matrix crystals were removed from the surface of the plate by rinsing with deionized H₂O. The hydrated slides were UV irradiated for 5 minutes and subsequently dried. FIG. 41 shows superposition of the fluorescence and MALDI TOF MS images of a section of the array produced by the above method. The irregular-shape spots labeled “f” are 635 nm Cy5 spots detected by fluorescence at 5 micron resolution. The square pixel-like spots labeled “m” are detected by mass spectrometry in the 1060 m/z mass channel. A single fluorescent spot without the corresponding MS signal (the signal is below threshold) is labeled with an asterisk. The fluorescence and MS images are intentionally offset to show the spot correlation. Analysis of the two images shows discrete spots, which are comparable to the size of individual microwells. The data indicates that: (i) loading of the solid phase crystals of MALDI matrix into microwells does not displace beads from the wells; (ii) the presence of matrix crystals in the wells does not interfere with the fluorescence detection and (iii) the resolution of mass spectrometry detection is similar to the resolution of the fluorescence scan. The size of spots detected by mass spectrometry indicates that the peptide analyte is localized within individual microwells.

### Example 17

Fabrication of an array of microspots from a library of microbeads with ten populations of beads, each bead population carrying a single peptide analyte.

The analytes are polypeptides of different molecular weight. The peptide sequences are: QPRDVTR (871 Da), DIEHNR (783 Da), DIERNR (802 Da), MIGGAGGRIR (987 Da), MIGGEGGRIR (1045 Da), MIGGIGGRIR (1029 Da), MIGGSGGRIR (1003 Da), MIGGPGGRIR (1013 Da), MIGGTGGRIR (1017 Da), MIGGRGGRIR (1072 Da). Each polypeptide is conjugated to microbeads via a photolabile linker and each microbead is conjugated to a single polypeptide. Thus, ten populations of beads are prepared. All beads are mixed and deposited on a microwell array plate. The MALDI matrix is applied in the solid form as described in the previous example. The analytes are eluted by UV irradiation and the fabricated array is measured by mass spectrometry. FIGS. 42A-42J show a series of images with each image recorded in a channel corresponding to the molecular weight of one of the analytes, as follows: FIG. 42A: 871 Da; FIG. 42B: 783 Da; FIG. 42C: 802 Da; FIG. 42D: 987 Da; FIG. 42E: 1045 Da; FIG. 42F: 1029 Da; FIG. 42G: 1003 Da; FIG. 42H: 1013 Da; FIG. 42I: 1017 Da; FIG. 42J: 1072 Da. The images reflect distribution of beads on the microwell plate and show that individual spots are localized and do not overlap. In fact, mass spectra recorded from each spot usually show a single strong peak corresponding to the analyte specific for the particular bead.

### Example 18

Sequencing of the peptide analyte directly on the microwell array plate using MALDI TOF-TOF mass spectrometry.

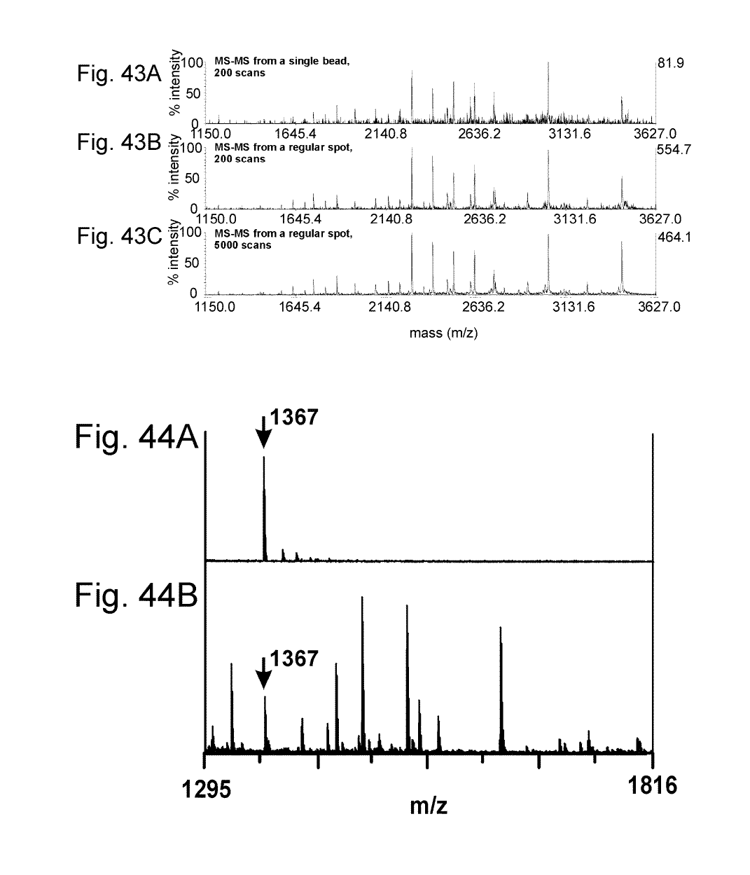

The array of microspots was prepared as described in Example 1. FIG. 43A, FIG. 43B and FIG.43C show the tandem MALDI TOF-TOF mass spectra of a 3483 Da polypeptide recorded from a single spot on a microarray produced from individual microbeads with an average of 200 scans per spectrum (FIG. 43A), from the regular MALDI stainless steel sample plate with an average of 200 scans per spectrum (FIG. 43B), and from the regular MALDI stainless steel sample plate with an average of 5000 scans per spectrum (FIG. 43C). The microarray data reflects spectra of the analyte produced by elution from individual microbeads, while in the control experiment the polypeptide solution was deposited on the regular stainless steel MALDI sample plate and mixed with the MALDI matrix solution using the dried droplet method. The spectra comparison reveals very similar pattern between the microarray and regular sample data, indicating that the microarray spots contain enough material to perform sequencing by mass spectrometry. Longer data acquisition performed on the regular sample plate (FIG. 43C) confirms that the majority of peaks are already detected in the microarray scan despite its lower signal-to-noise ratio.

### Example 19

Elution and detection of analytes of significantly different molecular weight that are conjugated to the same microbead.

The reaction was performed on a large group of identical microbeads deposited on the regular stainless steel MALDI target plate. Each bead was conjugated to a 1,367 Da polypeptide (HSV peptide, KQPELAPEDPED) and a larger (over 50,000 Da) HSV-tagged p53 protein, via the anti-HSV antibody covalently linked to beads. Two reactions were performed separately. In the first reaction, the beads were mixed with the low pH MALDI matrix solution. In the second reaction, the beads were first incubated with the solution of trypsin followed by the MALDI matrix solution. FIG. 44A and FIG. 44B show, respectively, mass spectra produced by low-pH elution from beads (FIG. 44A) and spectra produced by trypsin digestion followed by the low-pH elution (FIG. 44B). The first spectrum shows a single strong peak at 1367 Da due to the HSV peptide. The second spectrum shows multiple peaks arising from the fragments of trypsin-digested p53 as well as digested anti-HSV antibody. Importantly, the 1367 Da peak due to the HSV peptide, which is resistant to proteolysis, is also detected in the second spectrum.

### Example 20

Re-imaging of an array of microspots by MALDI TOF MS scanning

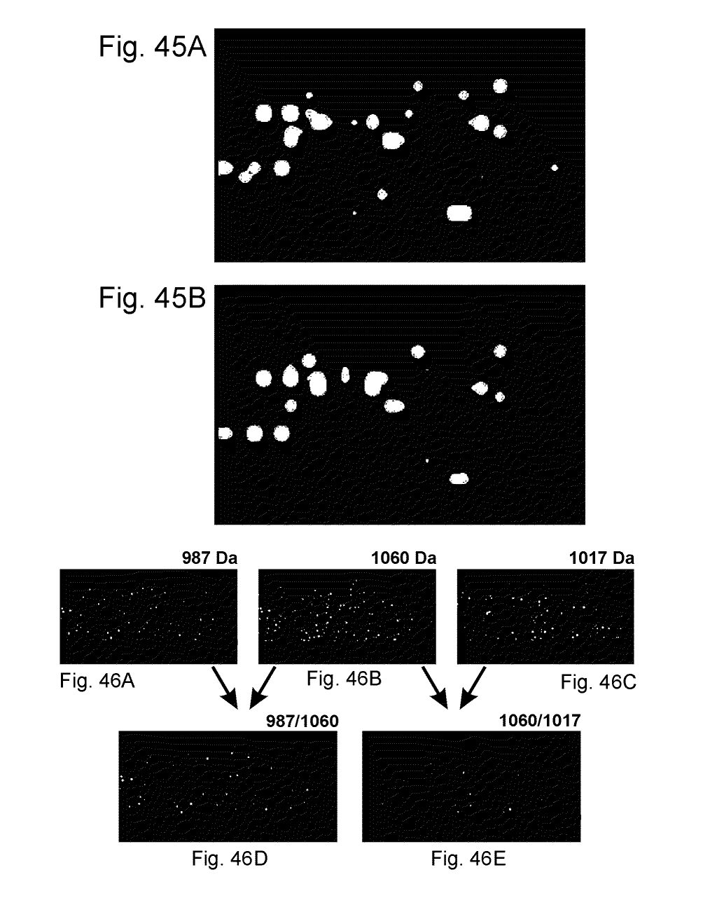

The reaction is performed as described in Example 1. The polypeptide of MW 3483 was eluted from beads and deposited on the surface of a microwell array plate (not inside the microwells) by application of a low-pH MALDI matrix solution. The MALDI TOF MS imaging was performed as previously described. Unlike Example 1, two MALDI TOF MS images were acquired from the same area by performing consecutive imaging using the identical data acquisition parameters. FIG. 45A and FIG. 45B show the images recorded during the first (FIG. 45A) and second (FIG. 45B) scans. The two images are very similar indicating that the analyte consumption by mass spectrometry in the first scan does not result in the complete depletion of the sample, thus preserving enough material and MALDI matrix to be detected in the subsequent scan. This result indicates that a microarray produced by the disclosed methods can be measured at least twice by the MALDI mass spectrometry imaging methods. For example, it allows to microarray to be re-measured after the first scan with different settings (e.g., different mass range or different spectral resolution) or by a different MS method (e.g. linear, reflector or MS-MS).

### Example 21

### MALDI TOF MS Imaging of a Microarray

The microarray MSI measurement is performed on ABI 4800 MALDI TOF/TOF mass spectrometer (AB Sciex, Foster City Calif.) equipped with the 4000 Series Explorer™ software. The image acquisition is performed using the 4000 Series Imaging software available in the public domain (www.maldi-msi.org). The image is collected in the MS reflector positive mode in the 650-3,500 Da mass range. The sampling bin size is 0.5 ns. The number of acquisition laser shots per spot is 100. The laser position remains fixed within a particular spot during the data acquisition. The rectangular area within the microarray selected for the imaging experiment is determined by the [x1,y1-x2, y2] set of coordinates, which are entered either manually or interactively within the 4000 Series Imaging software. The raster distance is set to 40 μm in both x and y directions. The microarray scan comprises stepwise displacement of the instrument sample plate with the mounted microarray slide by the raster distance with the data acquisition performed at each position. The data is collected and stored in the Analyze 7.5 format.

MS Image Data Analysis. Array scans produced by MALDI TOF mass spectrometry imaging are analyzed using the program BioMap available in the public domain (www.maldi-msi.org). The array images showing distribution of a particular analyte on the microarray slide were produced by selecting the molecular weight of that analyte as the “mass channel” in the BioMap software. Normally, the position of the maximum of the analyte monoisotopic peak was selected as the appropriate mass channel. The intensity scale was manually adjusted in each case and the lower cut-off level for the spot display was selected to be approximately three times above the noise level. Thus, the positive spots in the microarray images, which are shown in white, are areas with the analyte signal at least three-fold above the noise level. The black background represents areas where the signal in the particular “mass channel” was below the threshold.

### Example 22

### Imaging of an MSI-Compatible Microarray by Fluorescence

Microwell array plates with the fluorescent analytes are scanned using a GenePix 4200A laser based microarray scanner (Molecular Devices, Sunnyvale, Calif.) at one or more excitation wavelengths at 488, 532, 594 and 635 nm depending on the fluorophore. The pixel resolution is set to 10 micron and in some cases to 5 micron. The signal is acquired from the bottom of the microwell plate through the fiber optic channels. In order to measure eluted analytes the microwell plate is scanned in the “upside down” configuration with signal acquired from the surface containing openings into microwells. The focus offset is set according to the manufacturer's manual, typically between 0 and 50 micron.

### Example 23

### Microarray Image Overlay

A bead library comprising two distinct populations of beads was created by manually mixing suspensions containing approximately 100 beads of each type. The first population of beads is conjugated to two photolabile polypeptides: MIGGAGGRIR (MW 987 Da) serving as the bead label (1130 in FIG. 11) and RPPGFSPFR (Bradykinin, MW 1060 Da) serving as the probe label (1136 in FIG. 11). The second population of beads is conjugated only to MIGGTGGRIR (MW 1017 Da) polypeptide serving as the bead label. The bead library was converted into an array of microspots as described in Materials and Methods. The microarray was imaged as described in Example 21. Three microarray images were produced for each mass channel: 987 Da, 1017 Da and 1060 Da. FIG. 46A, FIG. 46B, and FIG. 46C show the corresponding array images. FIG. 46D and 46E show overlay of images generated in the 987/1060 Da and 1017/1060 Da mass channels, respectively. The overlay of 987/1060 Da mass channels, which reflect analytes present on the same bead, shows significantly greater degree of spot overlap compared to the overlay of 1017/1060 Da mass channels, which reflect analytes present of different beads. The spot overlap observed for the 1017/1060 Da mass channels is due to extended migration of eluted analytes on the microwell array plate.

### Example 24

### Detection of Interaction Between Analytes

This example demonstrates the ability to detect interaction between two analytes on beads by analyzing microarray MSI data. The first approach is image overlay, which is based on detection of co-localization of analytes using coordinates of individual spots on a microarray, the second approach is scatter plot, which is not coordinate-based and compares the signal intensity measured in analyte-specific mass channels.

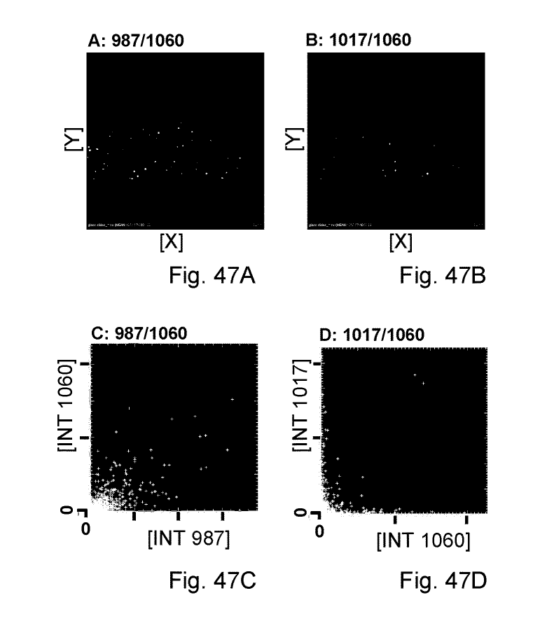

The microarray was prepared as described in Example 23 using a bead library comprising two populations of beads with 987/1060 Da and 1017 Da analytes, respectively. A significantly greater number of common spots was observed for the 987/1060 Da pair of analytes as shown in FIG. 47A compared to the 987/1017 Da pair of analytes as shown in FIG. 47B indicating that the overlap of the 987 and 1060 Da analytes is likely to be non-random. Note that each spot shown in FIG. 47A and FIG. 47B comprises several pixels. Furthermore, the scatter plot analysis demonstrates correlation in the intensity of 987 and 1060 Da peaks, but not in the intensity of 1017 and 1060 Da peaks. Specifically, a significantly greater number of data points in FIG. 47C has non-zero intensity measured in 987 and 1060 Da channels compared to data points in FIG. 47D that are measured in 1017 and 1060 Da channels.

### Example 25

### Visualization of the Microarray MSI Data in a Single Mass Channel and Continuous Mass Range

This example demonstrates visualization of the analyte distribution on a microarray using a single mass channel and a continuous mass range.

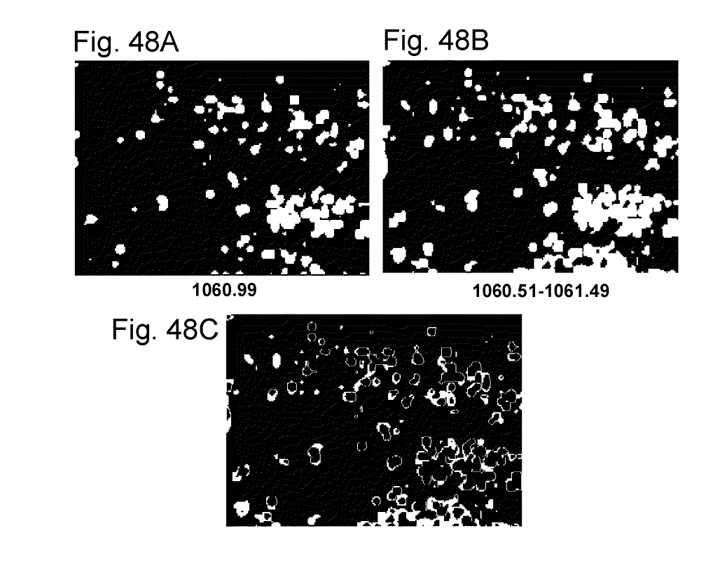

The microarray was fabricated as described in Materials and Methods and measured as described in Example 21. The measured analyte is Bradykinin (MW 1060 Da). FIG. 48A, FIG. 48B and FIG. 48C show microarray image of the analyte distribution measured in the 1060.99 m/z single mass channel (FIG. 48A) and in the 1060.51-1061.49 m/z continuous mass range (FIG. 48B). The image overlay (FIG. 48C) shows microarray areas that exhibit above the threshold signal only when the continuous mass range option is selected.

In an embodiment, a method for producing a random microarray is provided. First, microparticles binding at least one type of bound analyte can be distributed on a solid support, such that the individual microparticles are spatially separated. Next, at least one type of analyte is eluted from the microparticles and localized in the vicinity of the respective microparticles.

In an embodiment, a method for producing spatially distinct congruent microarrays located on the same solid support is provided. First, a plurality of microparticles is provided. In an embodiment, at least two different types of analytes are bound to the microparticles. In an embodiment, at least one type of analyte is fluorescent. Next, the microparticles are distributed on a solid support whereby the individual microparticles are spatially separated. Subsequently, at least one type of analyte from the microparticles is eluted, such that at least one type of fluorescent analyte remains bound to the microparticles. Finally, the released analytes are localized in the vicinity of their respective microparticles.

In an embodiment, a method for converting a library of beads to an array of analytes comprises positioning a plurality of beads having one or more analytes bound therein on a solid support in a spatially separated manner, causing the analytes to be released from the plurality of microparticles, and localizing the released analytes in discrete spots.

In an embodiment, a method for analyte analysis by mass spectrometry comprises converting a library of beads to an array of spots on a solid support, wherein each spot includes one or more analytes previously bound to a bead from the library of beads, and acquiring mass spectrometric data from the array of microspots according to a data acquisition protocol.

In an embodiment, a device for analysis of analyte-conjugated beads comprises a solid support having a plurality of microwells arranged in a regular grid, wherein the microwells are sized to accept one or more beads with analytes conjugated thereto, and wherein the microwells are positioned at a pre-determined distance from one another such that analytes released from the beads are localized in vicinity of respective beads.

All patents, patent applications, and published references cited herein are hereby incorporated by reference in their entirety. While the present disclosure has been described in connection with the specific embodiments thereof, it will be understood that it is capable of further modification. Furthermore, this application is intended to cover any variations, uses, or adaptations of the disclosure, including such departures from the present disclosure as come within known or customary practice in the art to which the disclosure pertains, and as fall within the scope of the appended claims

What is claimed is:

1. A composition comprising:
a solid support comprising a surface,
a plurality of beads located on the surface of the solid support, and
a plurality of spots located on the surface of the solid support wherein individual spots include analytes that were previously bound to individual beads from the plurality of beads.

2. The composition of claim 1 wherein the surface of the solid support is uneven, slanted or pitted.

3. The composition of claim 1 wherein the solid support is a microwell array plate.

4. The composition of claim 1 wherein the plurality of beads is a bead array in which at least some beads are separated by less than 1 mm.

5. The composition of claim 1 wherein the plurality of beads is a bead array that lacks positional encoding.

6. The composition of claim 1 wherein dimensions of the spots are less than 3-fold of dimensions of their respective beads.

7. The composition of claim 1 wherein dimensions of the spots are less than 2-fold of dimensions of their respective beads.

8. The composition of claim 1 wherein at least some beads are optically encoded.

9. The composition of claim 1 wherein at least some beads are fluorescent.

10. The composition of claim 1 wherein at least some beads are magnetic.

11. The composition of claim 1 wherein the analytes are peptide analytes and wherein at least some beads are capable of binding at least 10 fmol of a peptide analyte per bead.

12. The composition of claim 1 wherein the plurality of spots comprises at least 100 non-overlapping spots.

13. The composition of claim 1 wherein the plurality of spots comprises at least 10,000 non-overlapping spots.

14. The composition of claim 1 wherein the solid support is optically transparent.

15. The composition of claim 1 further comprising a plurality of nanoparticles located on the solid support.

16. A method of making the composition of claim 1, the method comprising:
providing a plurality of beads located on a surface of a solid support wherein individual beads are bonded to one or more analytes,
causing a release of at least some analytes from the plurality of beads, and
localizing the released analytes in discrete spots on the surface of the solid support while the plurality of beads remains positioned on the surface of the solid support.

17. The method of claim 16 wherein the releasing step comprises substantially simultaneously initiating a release of analytes from multiple beads.

18. The method of claim 16 wherein the releasing step comprises contacting the plurality of beads with an acidic compound.

19. The method of claim 16 wherein the releasing step comprises contacting the plurality of beads with a digestive compound.

20. The method of claim 16 wherein the releasing step comprises contacting the plurality of beads with an aerosol.

21. The method of claim 16 further comprising contacting the plurality of beads with a plurality of nanoparticles.

22. An analytical method, the analytical method comprising:
providing the composition of claim 1, and
analyzing by mass spectrometry the analytes within the plurality of spots.

23. The method of claim 22 wherein the mass spectrometry is desorption ionization mass spectrometry.

24. The method of claim 22 wherein the mass spectrometry is electrospray ionization mass spectrometry.

25. The method of claim 22 wherein the mass spectrometry is quantitative mass spectrometry.

26. The method of claim 22 wherein the mass spectrometry is imaging mass spectrometry.

27. The method of claim 22 wherein the analyzing step comprises fragmenting at least some of the analytes by mass spectrometry.

28. The method of claim 22 further comprising producing optical images of the plurality of beads and of the plurality of spots wherein the plurality of beads and the plurality of spots are imaged at different focal distances.

29. The method of claim 22 wherein at least some beads within the plurality of beads are optically encoded, the method further comprising using optical spectroscopy to measure optical codes of individual beads.

30. The method of claim 22 wherein the analyzing step comprises producing a mass spectrometric image of the plurality of spots, the method further comprising producing an optical image of the plurality of beads.
